(19) United States
(12) Patent Application Publication
(10) Pub. No.: US 2020/0308168 A1
(43) Pub. Date: Oct. 1, 2020

(54) FLUOROPHENYL SUBSTITUTED MUSCARINIC RECEPTOR LIGANDS WITH SELECTIVITY FOR M3 OVER M2

(71) Applicants: Friedrich-Alexander-Universität Erlangen-Nürnberg, Erlangen (DE); The Board of Trustees of the Leland Stanford Junior University, Stanford, CA (US); The Regents of the University of California, Oakland, CA (US)

(72) Inventors: Ashutosh BANERJEE, Berlin (DE); Amelie BARTUSCHAT, Hofheim (DE); Katrin EITEL, Berlin (DE); Peter GMEINER, Erlangen (DE); Markus HEINRICH, Langensendelbach (DE); Josefa HOFMANN, Darmstadt (DE); Harald HÜBNER, Heroldsbach (DE); Hannelore RAMPP, München (DE); Benjamin SCHAAKE, Nürnberg (DE); Brian KOBILKA, Palo Alto, CA (US); Roger SUNAHARA, San Diego, CA (US); Mary Joyce CLARK, San Diego, CA (US); Inbar FISH, Sunnyvale, CA (US); Brian SHOICHET, Kentfield, CA (US)

(73) Assignees: Friedrich-Alexander-Universität Erlangen-Nürnberg, Erlangen (DE); The Board of Trustees of the Leland Stanford Junior University, Stanford, CA (US); The Regents of the University of California, Oakland, CA (US)

(21) Appl. No.: 16/769,334

(22) PCT Filed: Dec. 3, 2018
(86) PCT No.: PCT/EP2018/083372

§ 371 Date: Jun. 3, 2020

(30) Foreign Application Priority Data
Dec. 4, 2017 (EP) 17205151.8

(51) Int. Cl. C07D 453/02; C07D 211/22; C07D 471/08; C07D 491/18

(52) U.S. Cl. CPC C07D 453/02; C07D 491/18; C07D 471/08; C07D 211/22

(57) ABSTRACT

The present invention relates to fluorophenyl substituted muscarinic receptor ligands with selectivity for M3 over M2 and to the use of these compounds in the treatment of various diseases such as asthma, chronic obstructive pulmonary disease (COPD), bronchopulmonary dysplasia (BPD) and urinary incontinence.

### FIELD OF THE INVENTION

The present invention relates to fluorophenyl substituted muscarinic receptor ligands with selectivity for M3 over M2 and to the use of these compounds in the treatment of various diseases such as asthma, chronic obstructive pulmonary disease (COPD), bronchopulmonary dysplasia (BPD) and urinary incontinence.

### BACKGROUND OF THE INVENTION

Muscarinic receptors are members of the class of G-protein coupled receptors (GPCRs). As they are involved in many physiological and pathophysiological processes, GPCRs are one of the most important groups of targets for the development of drugs. The muscarinic acetylcholine receptors can be divided into the five subtypes M1 to M5 with the M2 receptors being mainly expressed in heart tissue and the M3 subtype which can preferentially be found in the lung. Because excitatory M3 receptors are expressed on airway smooth muscles and mucosal glands mediating mucus secretion and bronchoconstriction, muscarinic M3 antagonists are used for the treatment of lung diseases including asthma, the chronic obstructive pulmonary disease (COPD) and bronchopulmonary dysplasia (BPD). Further indications include the treatment of urinary incontinence.

The development of subtype selective antagonists within the muscarinic receptor family presents a major challenge for medicinal chemistry. As the orthosteric binding sites of all muscarinic receptors show very high similarity, the development of highly subtype selective ligands for a distinct receptor is difficult. Very recently, the structural details of both receptors have been resolved, when the X-ray crystal structures of M2 and M3 in complex with the nonselective ligands QNB and tiotropium, respectively, were published. Key interactions include a hydrogen bond between Asn6.52 and the hydroxyl group and the ester moiety of the ligand. Similarly, the quarternary ammonium and the protonated tertiary amine of tiotropium and QNB, respectively, are stabilized by an ionic interaction with Asp3.32 and enclosed by an aromatic cage, built by the side chains of the four tyrosine residues Tyr3.33, Tyr6.51, Tyr7.39 and Tyr7.43 (according to Ballesteros-Weinstein nomenclature). The aryl moieties of the references form similar hydrophobic interactions with Thr5.39, Tyr3.33, Trp4.57 in both crystal structures, whereas the M2 and M3 differ in the interaction of the amino acid at position 181(M2) and 225 (M3), respectively, in the extracellular loop2 (ECL2). In the M2 receptor, Phe181 occupies more space than the corresponding amino acid Leu, which is located at position 225 in the M3 receptor.

Certain muscarinic receptor antagonists have been described in WO 95/021820 A1, EP 2 119 716 A1, WO 2005/067537 A2, WO 2006/005057 A2 and WO 2004/048373 A1.

### SUMMARY OF THE INVENTION

In view of the known muscarinic receptor M3 antagonists, it is an object of the present invention to provide novel and/or improved M3 antagonists, particularly more potent and/or more selective antagonists. In particular, muscarinic receptor M3 antagonists having a high selectivity for the M3 subtype over the M2 subtype are desired.

The present inventors have found that compounds of formula (I) are very useful as ligands having a high selectivity for muscarinic receptor M3, in particular over M2. Thus, the present invention relates to a compound of formula (I), optionally in the form of a pharmaceutically acceptable salt, solvate, polymorph, tautomer, racemate, enantiomer or diastereomer or mixture thereof,

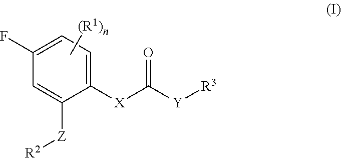

wherein
X is selected from —N(H)— and —C(H)(OH)—;
Y is selected from —O— and —N(H)—;
Z is selected from a bond and -(ethynylene)-;
R¹ is selected from -halogen, —CN, —CF₃, —C₁₋₆-alkyl, —OH and —O—C₁₋₆-alkyl; preferably, R¹ is —F;
n is an integer of 0 to 2;
R² is selected from -(optionally substituted aryl), -(optionally substituted heteroaryl) and -(optionally substituted cycloalkyl), wherein the one or more optional substituent(s) of the aryl, heteroaryl and cycloalkyl are selected from -halogen, —CN, —CF₃, —C₁₋₆-alkyl, —OH, —O—C₁₋₆-alkyl, —NH₂, —N(H)(C₁₋₆-alkyl) and —N(C₁₋₆-alkyl)₂; preferably the one or more optional substituent(s) of the aryl, heteroaryl and cycloalkyl are selected from -halogen, —CN, —OMe and -methyl;
R³ is a -(non-aromatic, optionally bridged, optionally substituted heterocyclic ring having 4 to 7 ring carbon atoms and 1 to 3 heteroatoms selected from N, O and S, including at least one nitrogen, in the ring, and optionally 1 to 3 carbon atoms and 0 to 2 heteroatoms selected from N, O and S in the bridge), wherein the bridge may be saturated or unsaturated and may contain an oxirane moiety, if the heterocyclic ring is a monocyclic ring without any bridge, the heterocyclic ring may be bonded to Y via a methylene group, and
wherein the optional substituent is selected from -halogen, —(C₁₋₆-alkyl) and —OH;

In addition, the present invention relates to a pharmaceutical composition comprising the compound of formula (I) and to a compound of this formula for use in the treatment, amelioration and/or prevention of asthma, chronic obstructive pulmonary disease (COPD), chronic obstructive lung disease, chronic bronchial asthma, chronic bronchitis, bronchopulmonary dysplasia (BPD), chronic airway obstruction, fibroid lung, diffuse panbronchiolitis, bronchiectasis, chronic respiratory obstruction, pulmonary fibrosis, pulmonary emphysema and allergic rhinitis, idiopathic interstitial pneumonia, urinary incontinence and/or cognitive disorders.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The present invention also relates to each of the compounds described herein in non-salt form or in the form of a pharmaceutically acceptable salt.

Throughout this specification and the claims which follow, unless the context requires otherwise, the word “comprise”, and variations such as “comprises” and “comprising”, will be understood to imply the inclusion of a stated integer or feature but not the exclusion of any other integer or feature.

Certain groups in the compounds of the present invention contain “one or more” substituents. It is to be understood that these substituents may be independently selected and thus be the same or different in each occurrence. Unless otherwise specified, the “one or more” substituents are preferably one, two, three or four substituents, more preferably, one, two or three substituents, even more preferably, one or two substituents, and most preferably one substituent.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Definitions

The term “preferably” is used to describe features or embodiments which are not required in the present invention but may lead to improved technical effects and are thus desirable but not essential.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

A number of compounds are described herein by reference to their structural formula and/or their chemical name, such as the IUPAC name. In case of discrepancies between the structural formula and the chemical name, the present invention expressly relates to the compounds as referred to by the structural formula as well as by the chemical name.

The term “halogen” preferably refers to F, Cl, Br or I, preferably F or Cl, more preferably F. The term “halide” preferably refers to F⁻, Cl⁻, Br⁻ or I⁻, preferably Cl⁻ or Br⁻.

The term “alkyl” refers to a saturated straight or branched carbon chain, which, unless otherwise defined, preferably has 1 to 6 carbon atoms, more preferably 1 to 4 carbon atoms, even more preferably 1 to 3 carbon atoms, still more preferably 1 or 2 carbon atoms or only 1 carbon atom. Unless otherwise specified, the “alkyl” may be substituted with one or more fluorine.

The term “cycloalkyl” preferably refers to a monocyclic five- or six-membered ring which consists only of carbon and hydrogen, such as cyclopentyl and cyclohexyl, preferably cyclohexyl. Unless otherwise specified, the “cycloalkyl” may be substituted with one or more fluorine.

The term “aryl” preferably refers to an aromatic monocyclic ring containing 5 or 6 carbon atoms, an aromatic bicyclic ring system (wherein one or both rings are aromatic) containing 9 carbon atoms such as 3H-indenyl, or an aromatic bicyclic ring system containing 10 carbon atoms. The term “aryl” more preferably refers to an aromatic monocyclic ring containing 5 or 6 carbon atoms or an aromatic bicyclic ring system containing 10 carbon atoms, more preferably an aromatic monocyclic ring containing 5 or 6 carbon atoms. Examples are phenyl or naphthyl, preferably phenyl.

The term “heteroaryl” preferably refers to a five- or six-membered aromatic ring, wherein one or more of the carbon atoms in the ring have been replaced by 1, 2, 3, or 4 (for the five-membered ring) or 1, 2, 3, 4, or 5 (for the six-membered ring) of the same or different heteroatoms, wherein the heteroatoms are selected from O, N and S. The term “heteroaryl” covers both monocyclic ring systems as well as aromatic bicyclic ring systems. Bicyclic “heteroaryl” groups may, e.g., be benzannulated derivatives of a five- or six-membered aromatic ring, wherein one or more of the carbon atoms in the ring have been replaced by 1, 2, 3, or 4 (for the five-membered ring) or 1, 2, 3, 4, or 5 (for the six-membered ring) of the same or different heteroatoms, whereby the heteroatoms are selected from O, N and S. Examples include thienyl, furanyl, thiazolyl, pyridyl, pyrimidyl, benzothienyl, benzofuranyl and indenyl, wherein 2-thienyl, 3-thienyl, 2-furanyl, 3-furanyl, 5-thiazolyl, 4-pyridyl, 5-pyrimidyl, 2-benzo[b]thiophenyl, 3-benzo[b]thiophenyl, and 2-benzofuranyl are preferred. Particularly preferred examples are 2-thienyl, 3-thienyl, 2-furanyl, 3-furanyl, 4-pyridyl, 5-pyrimidyl, 2-benzo[b]thiophenyl and 2-benzofuranyl. It is to be understood that each of these examples may also be substituted with one or more selected from -halogen, —CN, —CF₃, —C₁₋₆-alkyl, —OH, —O—C₁₋₆-alkyl, —NH₂, —N(H)(C₁₋₆-alkyl) and —N(C₁₋₆-alkyl)₂; preferably with one or more selected from -halogen, —CN, —OMe and -methyl.

The “non-aromatic, optionally bridged, optionally substituted heterocyclic ring having 4 to 7 ring carbon atoms and 1 to 3 heteroatoms selected from N, O and S, including at least one nitrogen, in the ring, and optionally 1 to 3 carbon atoms and 0 to 2 heteroatoms selected from N, O and S in the bridge” preferably contains at most one unsaturated bond such as a C═C double bond. The term “bridged” preferably means that the “bridge”, which is a divalent group comprising optionally 1 to 3 carbon atoms and 0 to 2 heteroatoms selected from N, O and S, is attached to two non-adjacent atoms in the main ring (“the ring”). The one or more substituents may be at any position of the main ring (“the ring”) or bridge. Preferably, the substituents are at the nitrogen in the ring. It is to be understood that each of these examples may also be substituted with one or more selected from -halogen, —(C_{1-6—}alkyl) and —(OH). The term “oxirane” preferably refers to a group represented by

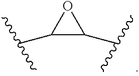 [SMILES: *C1OC1*]

The term “monocyclic ring without any bridge” preferably refers to a monocyclic ring in which only one ring is present such as in pyrrolidine, imidazolidine, piperidine, piperazine, morpholine and thiomorpholine. The monocyclic ring without any bridge is more preferably piperidine, such as 4-piperidyl.

The term “physiologically acceptable anion”, which may be represented by B⁻, preferably refers to an anion selected from halides (such as, e.g., chloride, bromide or iodide); hydroxide; carbonate; hydrogencarbonate; sulfates (such as, e.g., sulfate or hydrogensulfate), phosphates (such as, e.g., phosphate, hydrogenphosphate, or dihydrogenphosphate), nitrate, perchlorate, borate, or thiocyanate as well as organic anions such as C₁₋₂₀-alkanoates (such as, e.g., formate, acetate, propionate, butyrate, pentanoate, hexanoate, heptanoate, octanoate, nonanoate, decanoate, undecanoate, oleate, stearate); lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate; sulfonates such as C₁₋₂₀-alkyl sulfonate (such as, e.g., methanesulfonate (mesylate), ethanesulfonate (esylate)); 2-hydroxyethanesulfonate (isethionate), C₆₋₁₀-aryl sulfonates (such as e.g. benzenesulfonate (besylate) and 2-naphthalenesulfonate (napsylate)), C₁₋₂₀-alkyl-C₆₋₁₀-aryl sulfonate (such as p-toluenesulfonate (tosylate)), and anions of acidic amino acids such as aspartate or glutamate. It is to be understood that the C₁₋₂₀-alkyl and C₆₋₁₀-aryl in C₁₋₂₀-alkanoate, C₁₋₂₀-alkyl sulfonate, C₆₋₁₀-aryl sulfonate and C₁₋₂₀-alkyl-C₆₋₁₀-aryl sulfonate are optionally independently substituted with one or more halogen, wherein the halogen is preferably selected from fluoride.

More specific anions include chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

The term “pharmaceutically acceptable salt” refers to a salt of a compound of the present invention. Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

Moreover, the scope of the invention embraces the compounds of formula (I) in the form of any polymorph. It is to be understood that such polymorphs of the compounds of the formula (1) also include polymorphs of pharmaceutically acceptable salts and solvates of the compounds of the formula (I).

The term “treatment” of a disorder or disease as used herein is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease). The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., lessening of symptoms) or complete response (e.g., disappearance of symptoms) of the subject/patient suffering from the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The “amelioration” of a disorder or disease as used herein is also well known in the art. For example, it may lead to a halt in the progression of the disorder or disease or a delay in the progression of the disorder or disease.

The term “prevention” of a disorder or disease as used herein is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of the compounds and compositions of the present invention before any clinical and/or pathological symptoms are diagnosed or determined or can be diagnosed or determined by the attending physician.

In the present invention, it is to be understood that the term “muscarinic receptor” refers to “muscarinic acetylcholine receptor”.

When the compounds of the present invention are provided in crystalline form, the structure can contain solvent molecules. The solvents are typically pharmaceutically acceptable solvents and include, among others, water (hydrates) or organic solvents. Examples of possible solvates include ethanolates and iso-propanolates.

Compounds having the Formula (I)

The present invention relates to a compound of formula (I), optionally in the form of a pharmaceutically acceptable salt, solvate, polymorph, tautomer, racemate, enantiomer or diastereomer or mixture thereof,

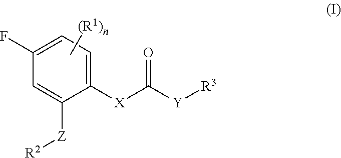

wherein
X is selected from —N(H)— and —C(H)(OH)—;
Y is selected from —O— and —N(H)—;
Z is selected from a bond and -(ethynylene)-;
R¹ is selected from -halogen, —CN, —CF₃, —C₁₋₆-alkyl, —OH and —O—C₁₋₆-alkyl, preferably —F;
n is an integer of 0 to 2;
R² is selected from -(optionally substituted aryl), -(optionally substituted heteroaryl) and -(optionally substituted cycloalkyl), wherein the one or more optional substituent(s) of the aryl, heteroaryl and cycloalkyl are selected from -halogen, —CN, CF₃, —OH, —O—C₁₋₆-alkyl, —NH₂, —N(H)(C₁₋₆-alkyl) and —N(C₁₋₆-alkyl)₂; preferably the one or more optional substituent(s) of the aryl, heteroaryl and cycloalkyl are selected from -halogen, —CN, —OMe and -methyl;
R³ is a -(non-aromatic, optionally bridged, optionally substituted heterocyclic ring having 4 to 7 ring carbon atoms and 1 to 3 heteroatoms selected from N, O and S, including at least one nitrogen, in the ring, and optionally 1 to 3 carbon atoms and 0 to 2 heteroatoms selected from N, O and S in the bridge), wherein the bridge may be saturated or unsaturated and may contain an oxirane moiety, if the heterocyclic ring is a monocyclic ring without any bridge, the heterocyclic ring may be bonded to Y via a methylene group, and
wherein the optional substituent is selected from -halogen, —(C₁₋₆-alkyl) and —OH;

The following compounds are preferably excluded:

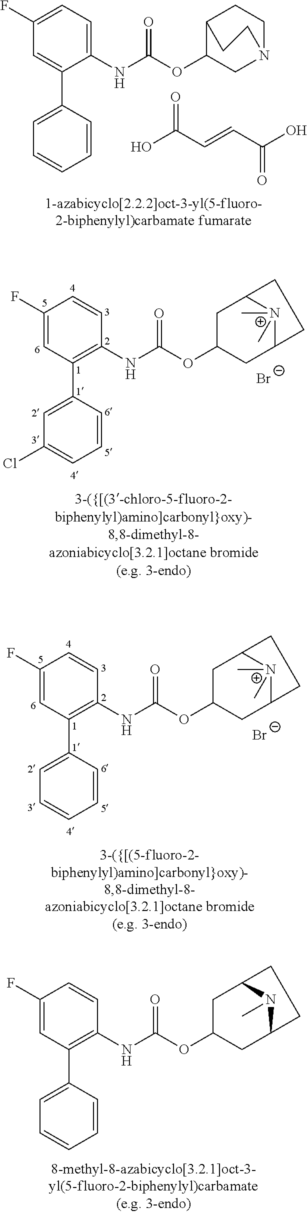

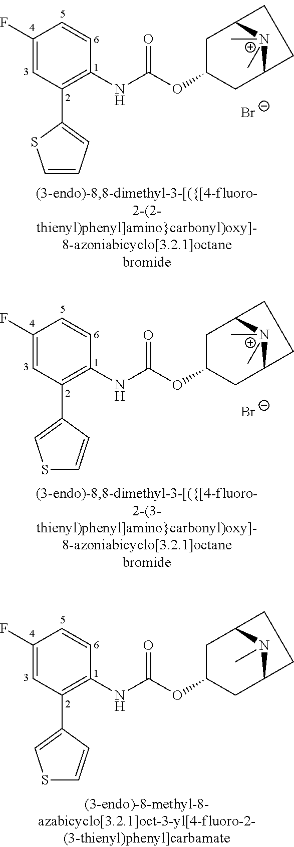 [SMILES: C[N+]1(C)[C@@H]2CC[C@H]1C[C@@H](OC(=O)Nc1ccc(F)cc1-c1cccs1)C2 ; [Br-] ; C[N+]1(C)[C@@H]2CC[C@H]1C[C@@H](OC(=O)Nc1ccc(F)cc1-c1ccsc1)C2 ; [Br-] ; CN1[C@@H]2CC[C@H]1C[C@@H](OC(=O)Nc1ccc(F)cc1-c1ccsc1)C2]

Preferably, both the compounds corresponding to the chemical name and the structural formula specified in the above table are excluded. Furthermore, any pharmaceutically acceptable salts of compounds specified in the above table and/or corresponding compounds of the compounds specified in the above table wherein the anion is replaced by a physiologically acceptable anion are optionally also excluded.

The compound of formula (I) may comprise one or two groups R¹, preferably one group R¹. Accordingly, if at least one R¹ is present, n is an integer of 1 or 2, preferably 1. If present, R¹ is preferably F. Alternatively, and preferably, the compound of formula (I) does not include group R¹, in which case n is 0.

R² is preferably selected from phenyl, thienyl, furanyl, thiazolyl, pyridyl, pyrimidyl, benzothienyl, benzofuranyl, indenyl and cyclohexyl, wherein phenyl, thienyl, furanyl, thiazolyl, pyridyl, pyrimidyl, benzothienyl, benzofuranyl, indenyl and cyclohexyl which are optionally substituted with one or more selected from -halogen, —CN, —CF₃, —C₁₋₆-alkyl, —OH, —O—C₁₋₆-alkyl, —NH₂, —N(H)(C₁₋₆-alkyl) and —N(C₁₋₆-alkyl)₂; preferably the one or more optional substituent(s) are selected from -halogen, —CN, —OMe and -methyl.

In cases where R² is benzothienyl or benzofuranyl, this group is preferably 2-benzo[b]thienyl or 2-benzo[b]furanyl.

More preferably, R² is selected from thienyl, furanyl, thiazolyl, pyridyl, pyrimidyl, benzothienyl, benzofuranyl and indenyl, wherein 2-thienyl, 3-thienyl, 2-furanyl, 3-furanyl, 5-thiazolyl, 4-pyridyl, 5-pyrimidyl, 2-benzo[b]thiophenyl, 3-benzo[b]thiophenyl, and 2-benzofuranyl are preferred. 2-Thienyl, 3-thienyl, 2-furanyl, 3-furanyl, 5-thiazolyl, 4-pyridyl, 5-pyrimidyl, 2-benzo[b]thiophenyl and 2-benzofuranyl are more preferred. Particularly preferred examples are 2-thienyl, 3-thienyl, 2-furanyl, 3-furanyl, 4-pyridyl, 5-pyrimidyl, 2-benzo[b]thiophenyl and 2-benzofuranyl. It is to be understood that the preferred examples of R² may also optionally be substituted with one or more selected from -halogen, —CN, —OMe and -methyl;

The optional substituent of the -(optionally substituted aryl), -(optionally substituted heteroaryl) and -(optionally substituted cycloalkyl) in R² is preferably selected from methyl, ethyl and halogen, more preferably halogen.

If R² is phenyl, it is preferably unsubstituted or substituted with one or two halogen, more preferably in para position such as 4-fluorophenyl, 4-chlorophenyl or 4-bromophenyl.

R³ is preferably a ring system selected from

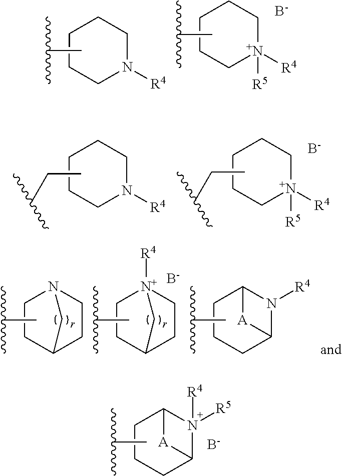

wherein

R⁴ is selected from H and C₁₋₆-alkyl;

R⁵ is selected from H and C₁₋₆-alkyl;

r is an integer from 1 or 2;

A is selected from 1,2-ethylene (—CH₂—CH₂—), 1,2-ethenylene (—CH═CH—) and 2,3-oxiranylene

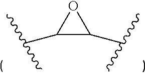

B⁻ is a physiologically acceptable anion.

More preferably, R³ is a ring system selected from

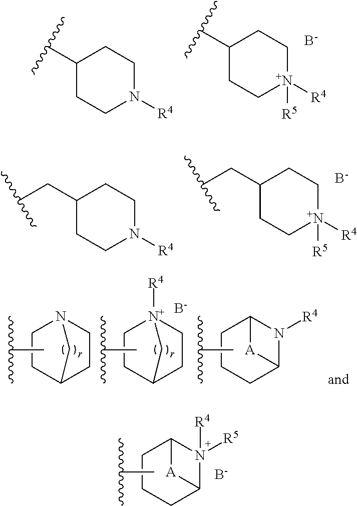

wherein R⁴, R⁵, r, A and B⁻ are as defined above.

Still more preferably, R³ is a ring system selected from

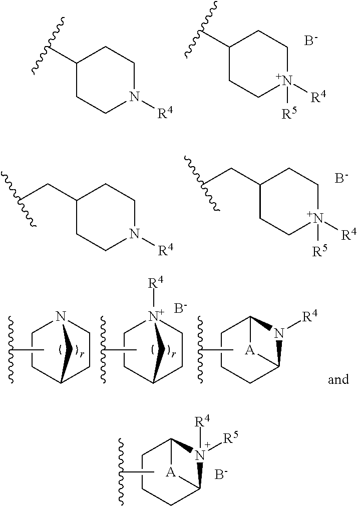

wherein R⁴, R⁵, r, A and B⁻ are as defined above.

Further preferably, the present invention does not relate to compounds wherein R² is -(optionally substituted phenyl), if Z is a bond, X is —N(H)—, Y is —O— and R³ is

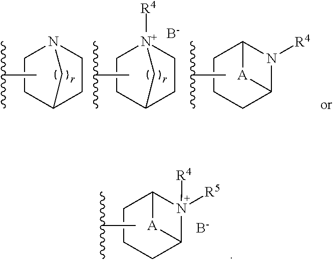

Further preferably, the present invention does not relate to compounds wherein R² is -(optionally substituted thienyl), if Z is a bond, X is —N(H)—, Y is —O— and R³ is

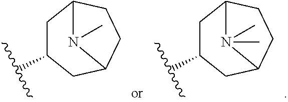

Further preferably, the present invention does not relate to compounds wherein R² is -(optionally substituted thienyl), if Z is a bond, X is —N(H)—, Y is —O— and R³ is

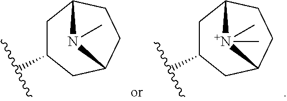

In the compounds of formula (I), Z is preferably a bond. Furthermore, in the compounds of formula (I), X is preferably NH and Y is preferably O.

In highly preferred compounds of formula (I),

R³ is

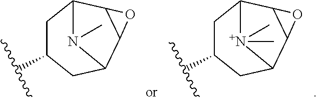

In particularly preferred compounds of formula (I), R³ is

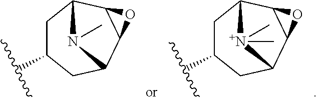

In such highly and particularly preferred compounds, one or more or each of the following conditions may apply: R² is -(optionally substituted heteroaryl), Z is a bond, X is —N(H)—, Y is —O—. In each of these cases, R³ is most preferably

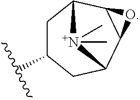 [SMILES: *[C@@H]1C[C@@H]2[C@H]3O[C@H]3[C@H](C1)[N+]2(C)C]

The present invention particularly relates to each of the compounds described in the examples section, either in non-salt form or in the form of a pharmaceutically acceptable salt.

Compounds according to formula (I) which are highly preferred are the following: piperidin-4-ylmethyl (5-fluoro-[1,1′-biphenyl]-2-yl)carbamate, piperidin-4-ylmethyl (2′-chloro-5-fluoro-[1,1′-biphenyl]-2-yl)carbamate, piperidin-4-ylmethyl (4′,5-difluoro-[1,1′-biphenyl]-2-yl)carbamate, piperidin-4-ylmethyl (4′-chloro-5-fluoro-[1,1-biphenyl]-2-yl)carbamate, piperidin-4-ylmethyl (4′-bromo-5-fluoro-[1,1′-biphenyl]-2-yl)carbamate, piperidin-4-ylmethyl (4′-cyano-5-fluoro-[1,1′-biphenyl]-2-yl)carbamate, piperidin-4-ylmethyl (5-fluoro-4′-methoxy-[1,1′-biphenyl]-2-yl)carbamate, piperidin-4-ylmethyl (3′,4′-dichloro-5-fluoro-[1,1′-biphenyl]-2-yl)carbamate, piperidin-4-ylmethyl (3′,4′,5,5′-tetrafluoro-[1,1′-biphenyl]-2-yl)carbamate, (1S,3R,4S)-quinuclidin-3-yl (S)-2-(5-fluoro[1,1′-biphenyl]-2-yl)-2-hydroxyacetate, (1S,3R,4S)-quinuclidin-3-yl (R)-2-(5-fluoro-[1,1′-biphenyl]-2-yl)-2-hydroxyacetate, (1S,3R,4S)-3-(((5-fluoro[1,1′-biphenyl]-2-yl)carbamoyl)oxy)-1-methylquinuclidin-1-ium trifluoroacetate, 1-(5-fluoro[1,1′-biphenyl]-2-yl)-3-((1S,3R,4S)-quinuclidin-3-yl)urea, (1S,3R,4S)-3-(3-(5-fluoro-[1,1′-biphenyl]-2-yl)ureido)-1-methylquinuclidin-1-ium trifluoroacetate, (1S,3R,4S)-quinuclidin-3-yl (4,5-difluoro-[2-phenyl]-phen-1-yl)carbamate, (1S,3R,4S)-quinuclidin-3-yl (3,4,5-trifluoro-[2-phenyl]-phen-1-yl)carbamate, (1S,3R,4S)-quinuclidin-3-yl (3′,4′-dichloro-5-fluoro-[1,1′-biphenyl]-2-yl)carbamate, (1S,3R,4S)-quinuclidin-3-yl (4-fluoro-2-(thiophen-2-yl)phenyl)carbamate, (1S,3R,4S)-3-(((4-fluoro-2-(thiophen-2-yl)phenyl)carbamoyl)oxy)-1-methylquinuclidin-1-ium formate, (1S,3R,4S)-quinuclidin-3-yl (2-(3-bromothiophen-2-yl)-4-fluorophenyl)carbamate, (1S,3R,4S)-quinuclidin-3-yl (4-fluoro-2-(3-methylthiophen-2-yl)phenyl)carbamate, (1S,3R,4S)-quinuclidin-3-yl (4-fluoro-2-(4-methylthiophen-2-yl)phenyl)carbamate, (1S,3R,4S)-quinuclidin-3-yl (4-fluoro-2-(5-methylthiophen-2-yl)phenyl)carbamate, (1S,3R,4S)-quinuclidin-3-yl (4-fluoro-2-(thiophen-3-yl)phenyl)carbamate, (1S,3R,4S)-3-(((4-fluoro-2-(thiophen-3-yl)phenyl)carbamoyl)oxy)-1-methylquinuclidin-1-ium formate, (1S,3R,4S)-quinuclidin-3-yl (4-fluoro-2-(5-methylthiophen-3-yl)phenyl)carbamate, (1S,3R,4S)-quinuclidin-3-yl (2-(3-bromofuran-2-yl)-4-fluorophenyl)carbamate, (1S,3R,4S)-quinuclidin-3-yl (4-fluoro-2-(5-methylfuran-2-yl)phenyl)carbamate, (1S,3R,4S)-quinuclidin-3-yl (4-fluoro-2-(furan-3-yl)phenyl)carbamate, (1S,3R,4S)-quinuclidin-3-yl (4-fluoro-2-(thiazol-5-yl)phenyl)carbamate, (1S,3R,4S)-quinuclidin-3-yl (4-fluoro-2-(pyridin-4-yl)phenyl)carbamate, (1S,3R,4S)-quinuclidin-3-yl (4-fluoro-2-(pyrimidin-5-yl)phenyl)carbamate, (1R,3R,5S)-8-methyl-8-azabicyclo[3.2.1]octan-3-yl (4-fluoro-2-(thiophen-2-yl)phenyl)carbamate, (1R, 3S,5S)-8-methyl-8-azabicyclo[3.2.1]oct-6-en-3-yl (4-fluoro-2-(thiophen-2-yl)phenyl)carbamate, (1R,2R,4S,5S,7S)-9-methyl-3-oxa-9-azatricyclo[3.3.1.0^{2.4}]nonan-7-yl (4-fluoro-2-(thiophen-2-yl)phenyl)carbamate, (1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate, (1S,3R,4S)-quinuclidin-3-yl (2-(benzo[b]thiophen-2-yl)-4-fluorophenyl)carbamate, (1S,3R,4S)-quinuclidin-3-yl (2-(benzo[b]thiophen-3-yl)-4-fluorophenyl)carbamate, (1S,3R,4S)-quinuclidin-3-yl (2-(benzofuran-2-yl)-4-fluorophenyl)carbamate, (1S,3R,4S)-quinuclidin-3-yl (4-fluoro-2-(1H-inden-3-yl)phenyl)carba mate, (1S,3R,4S)-quinuclidin-3-yl (4-fluoro-2-(phenylethynyl)phenyl)carbamate and (1S,3R,4S)-quinuclidin-3-yl (2-(cyclohexylethynyl)-4-fluorophenyl)carbamate. Further highly preferred examples include (1R,2R,4S,5S,7S)-7-(((4-Fluoro-2-(5-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-Fluoro-2-(4-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-Fluoro-2-(3-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-Fluoro-2-(thiophen-3-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(Benzo[b]thiophen-2-yl)-4-fluorophenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(Benzo[b]thiophen-3-yl)-4-fluorophenyl)carbamoyl)oxy)-9,9-dimethyl-3-oxa-9-azatricyclo[3.3.1.0^{2,4}]nonan-9-ium formate, and (1R,2R,4S,5S,7S)-9-Methyl-3-oxa-9-azatricyclo[3.3.1.0^{2,4}]nonan-7-yl (5-fluoro-[1,1′-biphenyl]-2-yl)carbamate.

Among these, more preferred examples of the compound of formula (I) are selected from piperidin-4-ylmethyl (5-fluoro-[1,1′-biphenyl]-2-yl)carbamate, piperidin-4-ylmethyl (4′,5-difluoro-[1,1-biphenyl)-2-yl)carbamate, piperidin-4-ylmethyl (4′-chloro-5-fluoro-[1,1′-biphenyl]-2-yl)carbamate, piperidin-4-ylmethyl (4′-bromo-5-fluoro-[1,1′-biphenyl]-2-yl)carbamate, piperidin-4-ylmethyl (3′,4′-dichloro-5-fluoro-[1,1]-biphenyl]-2-yl)carbamate, piperidin-4-ylmethyl (3′,4′,5,5′-tetrafluoro-[1,1-biphenyl]-2-yl)carbamate, (1S,3R,4S)-quinuclidin-3-yl (S)-2-(5-fluoro-[1,1′-biphenyl]-2-yl)-2-hydroxyacetate, (1S,3R,4S)-quinuclidin-3-yl (R)-2-(5-fluoro-[1,1′-biphenyl]-2-yl)-2-hydroxyacetate, (1S, 3R,4S)-3-(((5-fluoro-[1,1′-biphenyl]-2-yl)carbamoyl)oxy)-1-methylquinuclidin-1-ium trifluoroacetate, 1-(5-fluoro[1,1′-biphenyl]-2-yl)-3-((1S,3R,4S)-quinuclidin-3-yl)urea, (1S,3R,4S)-3-(3-(5-fluoro-[1,1′-biphenyl]-2-yl)ureido)-1-methylquinuclidin-1-ium trifluoroacetate, (1S,3R,4S)-quinuclidin-3-yl (4,5-difluoro-[2-phenyl]-phen-1-pcarbamate, (1S,3R,4S)-quinuclidin-3-yl (3,4,5-trifluoro-[2-phenyl]-phen-1-yl)carbamate, (1S,3R,4S)-quinuclidin-3-yl (3′,4′-dichloro-5-fluoro-[1,1-biphenyl]-2-yl)carbamate, (1S,3R,4S)-quinuclidin-3-yl (4-fluoro-2-(thiophen-2-yl)phenyl)carbamate, (1S,3R,4S)-3-(((4-fluoro-2-(thiophen-2-yl)phenyl)carbamoyl)oxy)-1-methylquinuclidin-1-ium formate, (1S, 3R,4S)-quinuclidin-3-yl (2-(3-bromothiophen-2-yl)-4-fluorophenyl)carbamate, (1S,3R,4S)-quinuclidin-3-yl (4-fluoro-2-(3-methylthiophen-2-yl)phenyl)carbamate, (1S,3R,4S)-quinuclidin-3-yl (4-fluoro-2-(4-methylthiophen-2-yl)phenyl)carbamate, (1S,3R,4S)-quinuclidin-3-yl (4-fluoro-2-(5-methylthiophen-2-yl)phenyl)carbamate, (1S,3R,4S)-quinuclidin-3-yl (4-fluoro-2-(thiophen-3-yl)phenyl)carbamate, (1S,3R,4S)-3-(((4-fluoro-2-(thiophen-3-yl)phenyl)carbamoyl)oxy)-1-methylquinuclidin-1-ium formate, (1S,3R,4S)-quinuclidin-3-yl (4-fluoro-2-(5-methylthiophen-3-yl)phenyl)carbamate, (1S,3R,4S)-quinuclidin-3-yl (2-(3-bromofuran-2-yl)-4-fluorophenyl)carbamate, (1S,3R,4S)-quinuclidin-3-yl (4-fluoro-2-(5-methylfuran-2-yl)phenyl)carbamate, (1S,3R,4S)-quinuclidin-3-yl (4-fluoro-2-(furan-3-yl)phenyl)carbamate, (1S,3R,4S)-quinuclidin-3-yl (4-fluoro-2-(pyridin-4-yl)phenyl)carbamate, (1S,3R,4S)-quinuclidin-3-yl (4-fluoro-2-(pyrimidin-5-yl)phenyl)carbamate, (1R,3R,5S)-8-methyl-8-azabicyclo[3.2.1]octan-3-yl (4-fluoro-2-(thiophen-2-yl)phenyl)carbamate, (1R,3S,5S)-8-methyl-8-azabicyclo[3.2.1]oct-6-en-3-yl (4-fluoro-2-(thiophen-2-yl)phenyl)carbamate, (1R,2R,4S,5S,7S)-9-methyl-3-oxa-9-azatricyclo[3.3.1.0^{2,4}]nonan-7-yl (4-fluoro-2-(thiophen-2-yl)phenyl)carbamate, (1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate, (1S,3R,4S)-quinuclidin-3-yl (2-(benzo[b]thiophen-2-yl)-4-fluorophenyl)carbamate and (1S,3R,4S)-quinuclidin-3-yl (2-(benzofuran-2-yl)-4-fluorophenyl)carbamate. Further more preferred examples include (1R,2R,4S,5S,7S)-7-(((4-fluoro-2-(5-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(4-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(3-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(thiophen-3-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-3-yl)-4-fluorophenyl)carbamoyl)oxy)-9,9-dimethyl-3-oxa-9-azatricyclo[3.3.1.0^{2,4}]nonan-9-ium formate, and (1R,2R,4S,5S,7S)-9-methyl-3-oxa-9-azatricyclo[3.3.1.0^{2,4}]nonan-7-yl (5-fluoro-[1,1′-biphenyl]-2-yl)carbamate.

Among these, even more preferred examples of the compound of formula (I) are more selected from piperidin-4-ylmethyl (5-fluoro-[1,1′-biphenyl]-2-yl)carbamate, piperidin-4-ylmethyl (4′-chloro-5-fluoro-[1,1-biphenyl]-2-yl)carbamate, piperidin-4-ylmethyl (4′-bromo-5-fluoro-[1,1′-biphenyl]-2-yl)carbamate, piperidin-4-ylmethyl (3′,4′-dichloro-5-fluoro-[1,1′-biphenyl]-2-yl)carbamate, piperidin-4-ylmethyl (3′,4′,5,5′-tetrafluoro-[1,1′-biphenyl]-2-yl)carbamate, (1S,3R,4S)-quinuclidin-3-yl (S)-2-(5-fluoro-[1,1′-biphenyl]-2-yl)-2-hydroxyacetate, (1S, 3R,4S)-3-(((5-fluoro[1,1′-biphenyl]-2-yl)carbamoyl)oxy)-1-methylquinuclidin-1-ium trifluoroacetate, (1S,3R,4S)-3-(3-(5-fluoro-[1,1′-biphenyl]-2-yl)ureido)-1-methylquinuclidin-1-ium trifluoroacetate, (1S,3R,4S)-quinuclidin-3-yl (4,5-difluoro-[2-phenyl]-phen-1-yl)carbamate, (1S,3R,4S)-quinuclidin-3-yl (3′,4′-dichloro-5-fluoro-[1,1′-biphenyl]-2-yl)carbamate, (1S,3R,4S)-quinuclidin-3-yl (4-fluoro-2-(thiophen-2-yl)phenyl)carbamate, (1S,3R,4S)-quinuclidin-3-yl (4-fluoro-2-(4-methylthiophen-2-yl)phenyl)carbamate, (1S,3R,4S)-quinuclidin-3-yl (4-fluoro-2-(5-methylthiophen-2-yl)phenyl)carbamate, (1S,3R,4S)-quinuclidin-3-yl (4-fluoro-2-(thiophen-3-yl)phenyl)carbamate, (1S,3R,4S)-3-(((4-fluoro-2-(thiophen-3-yl)phenyl)carbamoyl)oxy)-1-methylquinuclidin-1-ium formate, (1S,3R,4S)-quinuclidin-3-yl (4-fluoro-2-(5-methylthiophen-3-yl)phenyl)carbamate, (1S,3R,4S)-quinuclidin-3-yl (4-fluoro-2-(pyridin-4-yl)phenyl)carbamate, (1S,3R,4S)-quinuclidin-3-yl (4-fluoro-2-(pyrimidin-5-yl)phenyl)carbamate, (1R,3R,5S)-8-methyl-8-azabicyclo[3.2.1]octan-3-yl (4-fluoro-2-(thiophen-2-yl)phenyl)carbamate, (1R,3S,5S)-8-methyl-8-azabicyclo[3.2.1]oct-6-en-3-yl (4-fluoro-2-(thiophen-2-yl)phenyl)carbamate, (1R,2R,4S,5S ,7S)-9-methyl-3-oxa-9-azatricyclo[3.3.1.0^{2,4}]nonan-7-yl (4-fluoro-2-(thiophen-2-yl)phenyl)carbamate and (1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate. Further even more preferred examples include (1R,2R,4S,5S,7S)-7-(((4-fluoro-2-(5-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(4-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(3-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(thiophen-3-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-3-yl)-4-fluorophenyl)carbamoyl)oxy)-9,9-dimethyl-3-oxa-9-azatricyclo[3.3.1.0^{2,4}]nonan-9-ium formate, and (1R,2R,4S,5S,7S)-9-methyl-3-oxa-9-azatricyclo[3.3.1.0^{2,9}]nonan-7-yl (5-fluoro[1,1′-biphenyl]-2-yl)carbamate.

Among these, still more preferred examples of the compound of formula (I) are more selected from (1R,2R,4S,5S,7S)-9-methyl-3-oxa-9-azatricyclo[3.3.1.0^{2,4}]nonan-7-yl (4-fluoro-2-(thiophen-2-yl)phenyl)carbamate and (1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate. Further even more preferred examples include (1R,2R,4S,5S,7S)-7-(((4-fluoro-2-(5-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(4-methylthiophen-2-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((4-fluoro-2-(3-methylthiophen-2-yl)phenyl)carbamoyl)oxy)-9,9-dimethyl-3-oxa-9-azatricyclo[3.3.1.0^{2,4}]nonan-9-ium formate, (1R,2R,4S ,5S,7 S)-7-(((4-fluoro-2-(thiophen-3-yl)phenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate, (1R,2R,4S,5S,7S)-7-(((2-(benzo[b]thiophen-3-yl)-4-fluorophenyl)carbamoyl)oxy)-9,9-dimethyl-3-oxa-9-azatricyclo[3.3.1.0^{2,4}]nonan-9-ium formate, and (1R,2R,4S,5S,7S)-9-methyl-3-oxa-9-azatricyclo[3.3.1.0^{2,4}]nonan-7-yl (5-fluoro-[1,1′-biphenyl]-2-yl)carbamate.

Even more preferred are (1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate and (1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate.

It is to be understood that, in each of these specific examples, the anions such as trifluoroacetate and formate can optionally be replaced by any other physiologically acceptable anion. Furthermore, each of these specific examples can either be in non-salt form or in the form of a pharmaceutically acceptable salt.

Even more preferred are compounds of formula (I) represented by pharmaceutically acceptable salts of (1R,2R,4S,5S,7S)-7-(((4-Fluoro-2-(thiophen-2-yl)phenyl)carbamoyl)oxy)-9,9-dimethyl-3-oxa-9-azatricyclo[3.3.1.0^{2,4}]nonan-9-ium, such as (1R,2R,4S,5S,7S)-7-(((4-Fluoro-2-(thiophen-2-yl)phenyl)carbamoyl)oxy)-9,9-dimethyl-3-oxa-9-azatricyclo[3.3.1.0^{2,4}]nonan-9-ium formate.

The compounds of the present invention can be administered to a patient in the form of a pharmaceutical composition which can optionally comprise one or more pharmaceutically acceptable excipient(s) and/or carrier(s).

The compounds of the present invention can be administered by various well known routes, including oral, rectal, intragastrical, intracranial and parenteral administration, e.g. intravenous, intramuscular, intranasal, intradermal, subcutaneous, and similar administration routes. Oral, intranasal and parenteral administration are particularly preferred. Depending on the route of administration different pharmaceutical formulations are required and some of those may require that protective coatings are applied to the drug formulation to prevent degradation of a compound of the invention in, for example, the digestive tract.

Suitable excipients are known to the skilled person and can, e.g. be found in the Handbook of Pharmaceutical Excipients, published by the American Pharmaceutical Association, or in the “Remington: The Science and Practice of Pharmacy” by the University of the Sciences in Philadelphia which are herein incorporated by reference. Furthermore, suitable excipients and/or carriers can be found in “Pharmazeutische Technologie” 10^{th} edition, 2006, edited by Rudolf Voigt and published by the “Deutscher Apotheker Verlag Stuttgart”, which is herein incorporated by reference, e.g., on page 336.

It is to be understood that each of the atoms in the compounds of the present invention may be present in the form of any isotope. For example, one or more hydrogen may be 1H, 2H or 3H, one or more fluorine may be ¹⁸F or ¹⁹F, and one or more carbon may be ¹²C, ¹³C, or ¹⁴C.

The subject to be treated with the compounds of the present invention is preferably a mammal, more preferably a human.

The compounds of the present invention are particularly useful for treating, ameliorating, or preventing any conditions or diseases which are mediated by muscarinic acetylcholine receptor, in particular subtype M3 of the muscarinic acetylcholine receptor. In other words, the compounds of the present invention can be used in the treatment, amelioration or prevention of any condition or disease in which an antagonist of M3 muscarinic acetylcholine receptor can provide beneficial effects.

The compounds of the present invention and the pharmaceutical composition comprising one or more of the compounds of the present invention may be used for the preparation of a medicament.

The present invention also relates to the use of a compound having the formula (I) for the preparation of a medicament for treating, ameliorating or preventing asthma, chronic obstructive pulmonary disease (COPD), chronic obstructive lung disease, chronic bronchial asthma, chronic bronchitis, bronchopulmonary dysplasia (BPD), chronic airway obstruction, fibroid lung, diffuse panbronchiolitis, bronchiectasis, chronic respiratory obstruction, pulmonary fibrosis, pulmonary emphysema and allergic rhinitis, idiopathic interstitial pneumonia, urinary incontinence and/or cognitive disorders.

Examples of conditions or diseases which can be treated, ameliorated or prevented by the compounds according to the present invention include asthma, chronic obstructive pulmonary disease (COPD), chronic obstructive lung disease, chronic bronchial asthma, chronic bronchitis, bronchopulmonary dysplasia (BPD), chronic airway obstruction, fibroid lung, diffuse panbronchiolitis, bronchiectasis, chronic respiratory obstruction, pulmonary fibrosis, pulmonary emphysema and allergic rhinitis, idiopathic interstitial pneumonia, urinary incontinence and/or cognitive disorders.

Preferred conditions or diseases which can be treated, ameliorated or prevented by the compounds according to the present invention include urinary incontinence as well as lung diseases including asthma, the chronic obstructive pulmonary disease (COPD) and bronchopulmonary dysplasia (BPD).

The present inventors have surprisingly found that compounds of the present invention, which are represented by formula (I), are highly selective antagonists for subtype M3 of the muscarinic acetylcholine receptor while exhibiting reduced antagonistic activity for subtype M2 of the muscarinic acetylcholine receptor. Without wishing to be bound by theory, it is believed that the fluorine in the central phenyl ring in the compounds of the present invention contributes to this selectivity.

This effect is of significant importance in treating, ameliorating, or preventing any conditions or diseases which are mediated only by, or mainly by, subtype M3 of the muscarinic acetylcholine receptor. Undesired side effects may thus be reduced by using compounds according to the present invention.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention as indicated by the appended claims in any way.

### EXAMPLES

The following examples of compounds according to the present invention have been prepared and the binding affinities for the muscarinic receptor subtypes M2 and M3 as shown in Table 1 have been determined.

Biological Assay:

The compounds according to the present invention were investigated biologically by determination of the binding affinities for the muscarinic receptor subtypes M2 and M3 by radioligand competition binding experiments. For this purpose HEK cells were transiently transfected with the cDNA of the human receptor subtypes M2 and M3. Membranes of the cells were prepared to be incubated with the radioligand [³H]N-methyl-scopolamine and different concentrations of the test compound. After incubation at 37° C. membranes were harvested on glass fiber mats, free radioactivity was separated from bound radioactivity and the amount of bound radioligand was determined by scintillation measurement in a plate reader. Counts were transformed into competition binding curves by non-linear regression analysis and the resulting IC₅₀ values were transformed into Kᵢ values by applying the equation of Cheng and Prusoff. Mean Kᵢ values were derived from two to eight individual experiments each done in triplicate.

TABLE 1
Receptor building affinities for the muscarinic acetylcholine receptor subtypes M2 and M3 and structural details
of the compounds of the present invention.^{a}

| | |
|---|---|
| | type A |
| 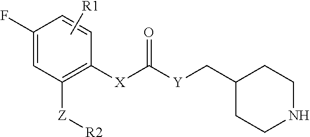 | |
| | type B |
| 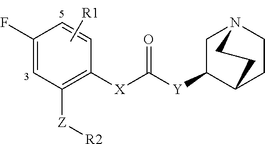 | |
| | type C |
| 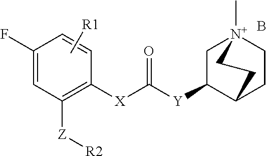 | |
| | type D |
| 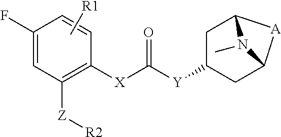 | |
| | type E |
| 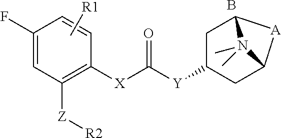 | |

| | | | | | | | | | Relative Kᵢ | |
|---|---|---|---|---|---|---|---|---|---|---|
| Compounds | | | | | | | | | values [nM]^{b} | |
| EXP | type | Z | R¹ | R² | X | Y | A | B | M2 | M3 |
| 1 | A | — | — | phenyl | NH | O | — | — | C | A |
| 2 | A | — | — | 2-chlorophenyl | NH | O | — | — | D | C |
| 3 | A | — | — | 4-fluorophenyl | NH | O | — | — | C | B |
| 4 | A | — | — | 4-chlorophenyl | NH | O | — | — | D | B |
| 5 | A | — | — | 4-bromophenyl | NH | O | — | — | D | B |
| 6 | A | — | — | 4-cyanophenyl | NH | O | — | — | D | D |
| 7 | A | — | — | 4-methoxyphenyl | NH | O | — | — | D | C |
| 8 | A | — | — | 3,4-dichlorophenyl | NH | O | — | — | B | A |
| 9 | A | — | — | 3,4,5-trifluorophenyl | NH | O | — | — | D | B |
| 10 | B | — | — | phenyl | (S)-CHOH | O | — | — | B | A |
| 11 | B | — | — | phenyl | (R)-CHOH | O | — | — | B | B |
| 12 | C | — | — | phenyl | NH | O | — | CF₃CO₂⁻ | B | A |
| 13 | B | — | — | phenyl | NH | NH | — | — | C | B |
| 14 | C | — | — | phenyl | NH | NH | — | CF₃CO₂⁻ | D | B |
| 15 | B | — | 5-fluoro | phenyl | NH | O | — | — | B | A |
| 16 | B | — | 3,5-difluoro | phenyl | NH | O | — | — | C | B |
| 17 | B | — | — | 3,4-dichlorophenyl | NH | O | — | — | B | A |
| 18 | B | — | — | 2-thienyl | NH | O | — | — | B | A |
| 19 | C | — | — | 2-thienyl | NH | O | — | HCO₂⁻ | C | A |
| 20 | B | — | — | 3-bromo-2-thienyl | NH | O | — | — | C | B |
| 21 | B | — | — | 3-methyl-2-thienyl | NH | O | — | — | C | B |
| 22 | B | — | — | 4-methyl-2-thienyl | NH | O | — | — | B | A |
| 23 | B | — | — | 5-methyl-2-thienyl | NH | O | — | — | B | A |
| 24 | B | — | — | 3-thienyl | NH | O | — | — | B | A |
| 25 | C | — | — | 3-thienyl | NH | O | — | HCO₂⁻ | C | A |
| 26 | B | — | — | 5-methyl-3-thienyl | NH | O | — | — | B | A |
| 27 | B | — | — | 3-bromo-2-furanyl | NH | O | — | — | C | B |
| 28 | B | — | — | 5-methyl-2-furanyl | NH | O | — | — | C | B |
| 29 | B | — | — | 3-furanyl | NH | O | — | — | C | B |
| 30 | B | — | — | 5-thiazolyl | NH | O | — | — | C | C |
| 31 | B | — | — | 4-pyridyl | NH | O | — | — | D | B |
| 32 | B | — | — | 5-pyrimidyl | NH | O | — | — | D | B |
| 33 | D | — | — | 2-thienyl | NH | O | 1,2-ethylene | — | C | A |
| 34 | D | — | — | 2-thienyl | NH | O | 1,2-ethylene | — | nd | nd |
| 35 | D | — | — | 2-thienyl | NH | O | 2,3-oxiranylene | — | C | A |
| 36 | E | — | — | 2-thienyl | NH | O | 2,3-oxiranylene | HCO₂⁻ | B | A |
| 37 | B | — | — | 2-benzthienyl | NH | O | — | — | C | B |
| 38 | B | — | — | 3-benzthienyl | NH | O | — | — | D | C |
| 39 | B | — | — | 2-benzofuranyl | NH | O | — | — | nd | nd |
| 40 | B | — | — | 3H-indenyl | NH | O | — | — | D | C |
| 41 | B | ethynylene | — | phenyl | NH | O | — | — | D | C |
| 42 | B | ethynylene | — | cyclohexyl | NH | O | — | — | D | C |
| 43 | E | — | — | 5-methyl-2-thienyl | NH | O | 2.3-oxiranylene | HCO₂⁻ | A | A |
| 44 | E | — | — | 4-methyl-2-thienyl | NH | O | 2,3-oxiranylene | HCO₂⁻ | A | A |
| 45 | E | — | — | 3-methyl-2-thienyl | NH | O | 2,3-oxiranylene | HCO₂⁻ | B | B |
| 46 | E | — | — | 3-thienyl | NH | O | 2,3-oxiranylene | HCO₂⁻ | A | A |
| 47 | E | — | — | 2-benzthienyl | NH | O | 2,3-oxiranylene | HCO₂⁻ | B | A |
| 48 | E | — | — | 3-benzthienyl | NH | O | 2,3-oxiranylene | HCO₂⁻ | D | C |
| 49 | D | — | — | phenyl | NH | O | 2,3-oxiranylene | — | C | A |

^{a}Ki values derived from radioligand competition binding experiments with membranes from HEK cells transiently expressing the
human receptor and the radioligand [³H]N-methylscopolamine performed by two to eight individual experiments each done in
triplicates.
^{b}Ki values are displayed divided in classes as follows: A: <1 nM, B: 1 to 20 nM, C: >20 to 200 nM, D: >200 nM.

Comparison of the receptor binding data of selected fluorophenyl substituted derivatives show the strong inducing effect of the fluorophenyl entity on subtype selectivity for M3 over M2 (Table 2). While EXP 18 and EXP 19 show an M3-receptor-selectivity of 39- and 27-fold, respectively, the corresponding defluoro analogues REF 2 and REF 3 gain only M3R selectivity of 7- and 6-fold. Similarly EXP 36 has a 5-fold better M3-receptor-selectivity compared to its defluoro analogue REF 1. Further studies regarding the relevance of the fluorine substituent for the selectivity of such compounds can be found in “Structure-guided development of selective M3 muscarinic acetylcholine receptor antagonists” by Liu et al., Proc Natl Acad Sci USA. 2018;115(47):12046-12050. The contents of this document and its supplementary information are hereby included by reference in their entirety.

TABLE 2
Receptor binding affinities for M2 and M3 of the reference compounds
REF1-REF 3.^{a}

| | |
|---|---|
| | REF 1 |
| 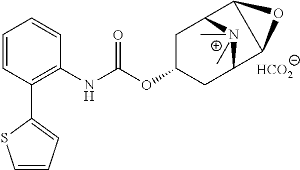 | |
| | REF 2 |
| 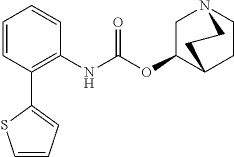 [SMILES: O=C(Nc1ccccc1-c1cccs1)O[C@H]1C[N@]2CC[C@H]1CC2] | |
| | REF 3 |
| 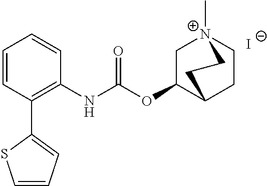 [SMILES: C[N@+]12CC[C@H](CC1)[C@@H](OC(=O)Nc1ccccc1-c1cccs1)C2 ; [I-]] | |

| | Relative Kᵢ values [nM]^{b} | |
|---|---|---|
| REF | M2 | M3 |
| 1 | A | A |
| 2 | B | A |
| 3 | B | A |

^{a}Ki values derived from radioligand competition binding experiments with
membranes from HEK cells transiently expressing the human receptor and
the radioligand [³H]N-methylscopolamine performed by two to eight individual
experiments each done in triplicates.
^{b}Ki values are displayed divided in classes as follows: A: <1 nM, B: 1 to
20 nM, C: >20 to 200 nM, D: >200 nM.

To investigate the kinetic properties of example compounds time-resolved radioligand binding experiments were performed with compound EXP 36 (kinetic binding experiment). As listed in Table 3 EXP 36 has a dissociation constant for binding at the M3 receptor of 0.00096 min⁻¹ corresponding to a very long residence time at M3. When comparing this constant to that of the reference atropine (0.023 min⁻¹) these data emphasize the strong binding of EXP 36 at M3. Additionally, the comparison of dissociation constants of EXP 36 for M3 and M2 (0.117 min⁻¹) reveals a 170-fold selectivity for M3 over M2 (Table 3).

TABLE 3
Binding kinetics of EXP 36 at the M2 and M3 receptor^{a}

| | dissociation constant (min⁻¹ ± SEM) | |
|---|---|---|
| | M2 receptor | M3 receptor |
| EXP 36 | 1.17 × 10⁻¹ ± 3.00 × 10⁻³ | 9.64 × 10⁻⁴ ± 4.21 × 10⁻⁴ |

^{a}Values are derived from radioligand kinetic binding experiments with membranes from CHO cells stably expressing the human M2 or M3 receptor and the radioligand [3H]N-methyl-scopolamine performed in three (for M2) or four (M3) individual experiments.

Synthesis:

The synthesis of embodiments can be performed starting with chemical precursors, which are available from common suppliers of fine chemicals, by name ACROS (via suppliers for fine chemicals like Fisher Scientific, Nidderau, Germany or VWR International, Darmstadt, Germany), Alfa Aeser (Karlsruhe, Germany), Activate Scientific (Rien, Germany), Sigma Aldrich (via Merck, Darmstadt, Germany), TCI Deutschland (Eschborn, Germany). All example compounds have been synthesized according to general procedures as described below:

General Procedure 1:

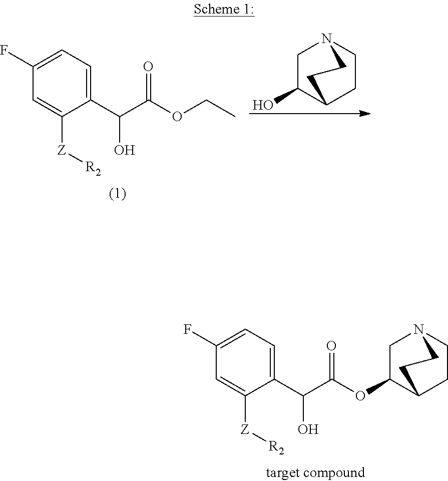

To a solution of R-(−)-3-quinuclidinol (1 eq) in dry dimethyl formamide (4 mL) was added under Ar-atmosphere K₂CO₃ (1.1 eq) and the suspension was stirred at room temperature for 1 h. Subsequently, a solution of (1) (0.36 eq), in dry dimethyl formamide (1 mL) was added, before the reaction mixture was stirred at 110° C. for 8 h. After the mixture was allowed to cool to room temperature, CH₂Cl₂ and water were added and the aqueous phase was extracted three times with CH₂Cl₂. The combined organic layers were washed twice with saturated, aqueous NaCl solution and dried over Na₂SO₄). After evaporation, the crude residue was purified by flash chromatography on silica gel to obtain the target compound.

General Procedure 2:

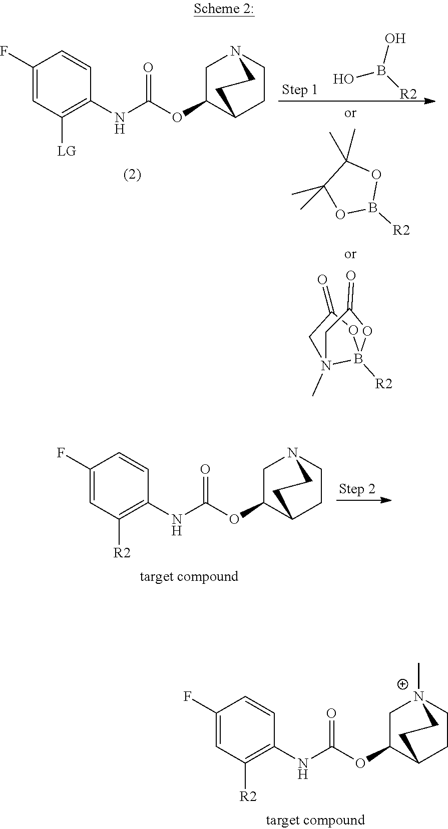

(LG denotes any suitable leaving group such as chloride, bromide or iodide)

Step 1: To a solution of (2) in 1,4-dioxane (4 mL) were added 1′1[bis(diphenylphosphino) ferrocene] dichloropalladium(II) (0.2 eq), Na₂CO₃ (8 eq) and one of the following regents heteroarylboronic acid or heteroarylboronic acid pinacol ester or heteroarylboronic MIDA ester (2 eq) in a microwave tube. After addition of H₂O (1 mL), the tube was sealed and the reaction mixture was stirred at 90° C. for 16 h. The mixture was allowed to cool to room temperature, was filtered through celite and MgSO₄ and the filter was repeatedly washed with ethyl acetate. After removing the solvent under reduced pressure, the crude residue was dissolved in saturated aqueous Na₂CO₃ solution. The aqueous layer was extracted three times with CHCl₃ and the combined organic layers were washed with saturated, aqueous NaCl solution and dried over Na₂SO₄). The organic solvent was removed under reduced pressure and the residue was purified by preparative HPLC to obtain the appropriate target compound.

Step 2: To a solution of the appropriate target compound in dry CH₂Cl₂ (2 mL) was added under argon atmosphere K₂CO₃ (3 eq) and methyl iodide (2.5 eq). The reaction was stirred at room temperature for 1 h. The solvent was removed under reduced pressure and the residue was purified by preparative HPLC to obtain a further target compound.

General Procedure 3:

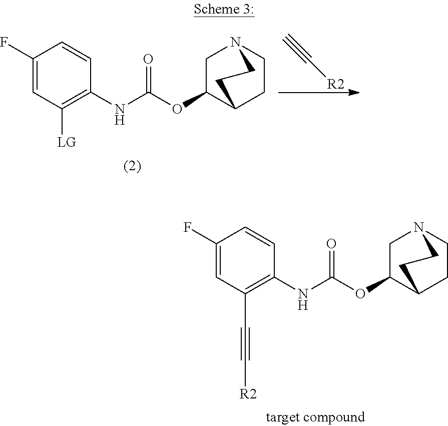

To a solution of (2) in dry toluene (4 mL) was added alkyl or arylacetylene (3 eq), copper iodide (0.1 eq), bis(triphenylphosphine)palladium(II) dichloride (0.2 eq) and triethylamine (3 eq) under argon atmosphere in a microwave tube. The tube was sealed and the reaction mixture was stirred for 16 h at 120° C. After allowing the reaction to cool to room temperature, saturated, aqueous NaHCO₃ solution was added and the aqueous layer was extracted three times with CHCl₃. The combined organic layers were washed with saturated, aqueous NaCl solution and dried over Na₂SO₄. After removing the organic solvent under reduced pressure the crude product was purified by preparative HPLC to obtain the target compound.

General Procedure 4:

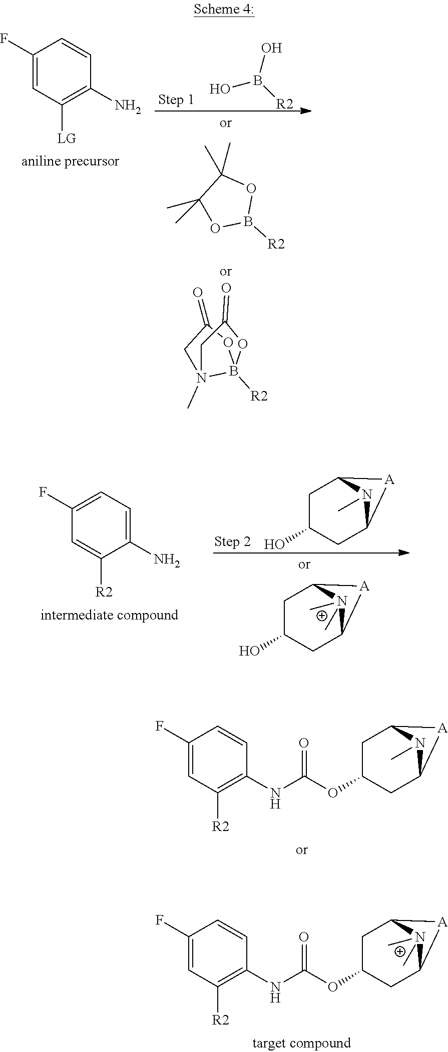

wherein LG denotes any suitable leaving group such as chloride, bromide or iodide.

Step 1: To a solution of aniline precursor in 1,4-dioxane (10 mL) were added 1′1[bis(diphenylphosphino)ferrocene]dichloropalladium(II) (0.02 eq), K₂CO₃ (2.5 eq) and one of the following reagents heteroarylboronic acid or heteroarylboronic acid pinacol ester or heteroarylboronic acid MIDA (6-methyl-1,3,6,2-dioxazaborocane-4,8-dione) ester (2 eq) in a microwave tube. After addition of H₂O (2.5 mL), the tube was sealed and the reaction mixture was stirred at 90° C. for 16 h. The mixture was allowed to cool to room temperature, was filtered through celite and MgSO₄ and the filter was repeatedly washed with ethyl acetate. After removing the solvent under reduced pressure, the residue was purified by column chromatography on silica gel to obtain the appropriate intermediate compound.

Step 2: To a solution of triphosgene in dry toluene (0.4 eq in 4.35 mL) was added a solution of the corresponding intermediate compound in dry toluene (0.65 mL) over 30 minutes under argon atmosphere at 0° C. The mixture was stirred at room temperature for additional 20 minutes and was refluxed for 16 h. After the reaction was allowed to cool to room temperature the solvent was removed under reduced pressure and the residue was distilled under vacuum conditions. The isolated isocyanate was added to a solution of the corresponding aliphatic alcohol compound in dry DMSO (3.5 mL) under argon atmosphere and was stirred at 40-90° C. for 16 h. The mixture was allowed to cool to room temperature, the solvent was removed in vacuum and the residue was purified by preparative HPLC to obtain the target compound.

General Procedure 5:

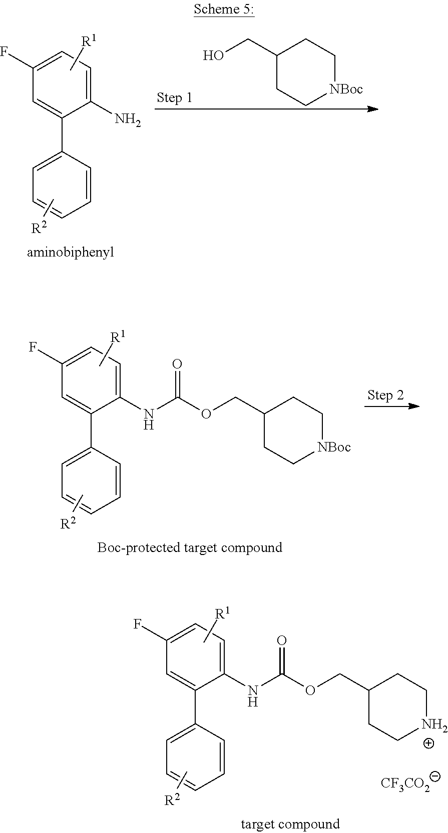

Step 1: To a solution of triphosgene (0.4 eq) in dry CH₂Cl₂ (1.0 mL) was added a solution of the aminobiphenyl (1 eq) in dry CH₂Cl₂ (0.5 mL) under argon atmosphere over a period of 20 minutes at 0° C. Subsequently, a solution of N,N-diisopropylethylamine (4 eq) in dry CH₂Cl₂ (0.5 mL) was added over 5 minutes and the mixture was stirred for additional 30 minutes at 0° C. A solution of N-Boc-4-piperidinemethanol (1.2 eq) in dry CH₂Cl₂ (0.5 mL) was added and the solution was stirred overnight at room temperature. The reaction mixture was diluted with CH₂Cl₂ (20 mL) and the organic phase was washed with a saturated aqueous solution of Na₂CO₃, a saturated aqueous solution of NaCl, and dried over Na₂SO₄. The solvent was removed under reduced pressure and the crude products were purified by column chromatography on silica gel to obtain the Boc-protected target compound.

Step 2: The corresponding Boc-protected target compound was stirred in trifluoroacetic acid in CH₂Cl₂ (10%, 1.0 mL) for 24 hours at room temperature. The reaction course was monitored by TLC. The solvent was removed under reduced pressure and the resulting trifluoroacetate was dried in vacuo to obtain the target compound.

General Procedure 6:

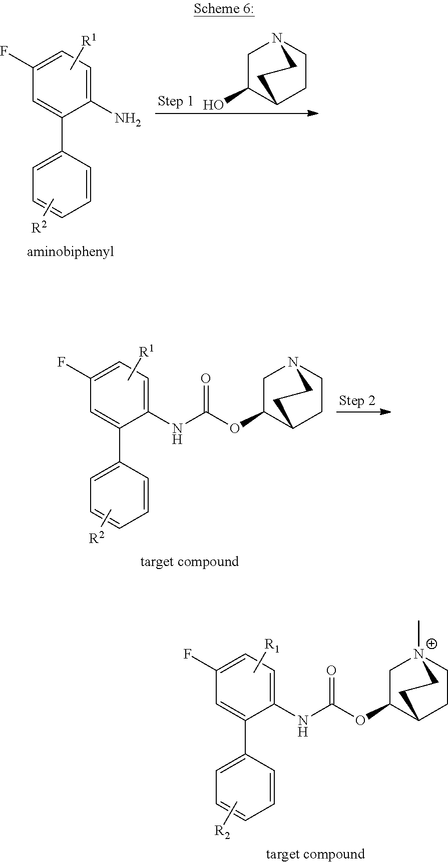

Step 1: A solution of triphosgene (0.4 equiv.) in dry CH₂Cl₂ (0.50 mL) was added to a solution the aminobiphenyl (1 eq) in dry CH₂Cl₂ (1.0 mL) under argon atmosphere over a period of 20 minutes at 0° C. The reaction mixture was stirred for another 15 minutes to complete the reaction, as monitored by TLC. The solvent was removed under reduced pressure and the residue was diluted in toluene (1.0 mL). Subsequently, a solution of (R)-quinuclidin-3-ol (1.2 eq) in toluene (0.5 mL) was added and the resulting mixture was stirred for 24 hours at 110° C. The reaction mixture was diluted with ethyl acetate (20 mL) and the organic layer was extracted three times with an aqueous HCI solution (1M). After adjusting the pH value of the combined aqueous phase to 9, using aqueous K₂CO₃, the aqueous phase was extracted with CHCl₃. After drying of the resulting organic phase over Na₂SO₄, the solvent was removed under reduced pressure and the crude product was purified by column chromatography on silica gel to obtain the appropriate target compound.

Step 2: The target compound (1.0 eq), methyl iodide (5.0 equiv.) and K₂CO₃ (3.0 equiv.) were stirred in dry CH₂Cl₂ for six hours at room temperature, as monitored by TLC. The solvent was removed under reduced pressure and the crude product was dried in vacuo and purified by preparative HPLC to obtain a further target compound.

Purification and Analytical Characterization:

Intermediates and target compounds were purified by chromatographic methods, usually by applying preparative HPLC using different chromatographic systems—mainly reversed phase columns—as described below (column 1, column 2):

Column 1: MACHAREY-NAGEL Varioprep VP 250/32 NUCLEODUR C18 HTec (250×32 mm, 5 μm, 32 mL/min)

Column 2: ZORBAX ECLIPSE XDB-C8 PrepHT (150×21.5 mm, 5 μm, 12 mL/min)

The analytical characterization of the final embodiments were performed by 1H-NMR at 400 MHz or 600 MHz, 13C-NMR at 150 MHz or 100 MHz, by mass spectrometry (ESI-MS) and by analytical HPLC applying one or two different chromatographic systems which are described below (system A, system B, system C):

System A: MeOH/H₂O+0.1% HCO₂H, 0.5 ml/min: 10%→10% MeOH in 3 min, 10%→100% MeOH in 15 min, 100%→100% in 6 min.

System B: acetonitrile/H₂O+0.1% TFA (trifluoroacetic acid), 0.5 mL/min: 10%→10% acetonitrile in 3 min, 10%→90% acetonitrile in 15 min, 90%→90% in 6 min.

System C: MeOH/H₂O+0.1% HCO₂H, 0.3 mL/min: 25%→100% MeOH in 6 min, 100%→100% MeOH in 2.5 min.

Synthesis of Precursors and Synthetic Intermediates:

### 2-Bromo-5-fluoro-1,1′-biphenyl (4)

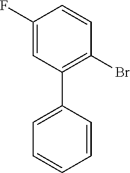 [SMILES: Fc1ccc(Br)c(-c2ccccc2)c1]

To a solution of 1-bromo-4-fluoro-2-iodobenzene (2.50 mL, 21.0 mmol) in dry toluene (20 mL) were added under Ar-atmosphere phenylboronic acid (0.3 eq), tetrakistriphenylphosphine-palladium(0) (0.03 eq) and an aqueous K₂CO₃ solution (3 M, 2 mL). The reaction was stirred at 120° C. for 16 h. The mixture was allowed to cool to room temperature, followed by the addition of saturated, aqueous NaHCO₃ solution and the aqueous layer was extracted three times with ethyl acetate. The combined organic layers were washed once with saturated, aqueous NaCl solution and dried over Na₂SO₄. After evaporation, the crude residue was purified by column chromatography on silica gel (n-hexane) to give 4 (1.48 g, 89%) as colorless oil.

HPLC (254 nm, System A): t_{R}=22.2 min.

### Ethyl 2-(5-fluoro-[1,1′-biphenyl]-2-yl)-2-hydroxyacetate (1)

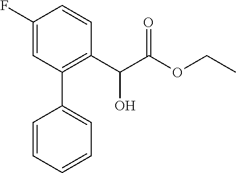 [SMILES: CCOC(=O)C(O)c1ccc(F)cc1-c1ccccc1]

A solution of 4 (600 mg, 2.39 mmol) in dry THF (tetrahydrofuran) (20 mL) was cooled to −78° C. Subsequently, n-buthyllithium (2.5 M solution in hexane, 2 eq) was added while the reaction temperature was maintained at a maximum of −75° C. The mixture was stirred at −78° C. for 25 min, followed by the addition of glyoxylic acid ethyl ester solution (50% in toluene, 4 eq, dissolved in dry THF (5 mL)), while the reaction temperature was maintained at a maximum of −70° C. The reaction was allowed to warm to −25° C. for three hours, followed by the addition of cold saturated, aqueous NH₄Cl solution. The suspension was stirred at room temperature for additional 30 min and then extracted three times with CHCl₃. The combined organic layers were washed twice with water and once with saturated, aqueous NaCl solution and dried over Na₂SO₄). After evaporation, the crude residue was purified by column chromatography on silica gel (n-hexane/ethyl acetate, 10:1) to give 1 (124 mg, 19%) as pale yellow oil.

HPLC (254 nm, System A): t_{R}=20.7 min; ESI-MS: 297.3 [M+Na]⁺

### (1S,3R,4S)-Quinuclidin-3-yl (2-bromo-4-fluorophenyl) carbamate (2)

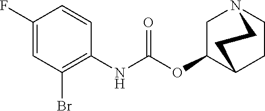

To a solution of 2-bromo-4-fluorobenzoic acid (1000 mg, 4.57 mmol) in dry toluene (8 mL) were added dry dimethyl formamide (10 μL) and SOCl₂ (0.5 mL) under argon atmosphere and the mixture was stirred under reflux conditions for 16 h. After the reaction was allowed to cool to room temperature, SOCl₂ was removed under reduced pressure. NaN₃ (2.5 eq) was added under argon atmosphere and the mixture was stirred at room temperature for 40 minutes and subsequently heated to 90° C. for 16 h.

After cooling to room temperature R-(−)-3-quinuclidinol (1.5 eq) was added under argon atmosphere and the reaction was stirred again at 120° C. for 2 h. After cooling to room temperature saturated aqueous NaHCO₃ solution was added and the aqueous layer was extracted three times with CHCl₃. The combined organic layer was washed with saturated aqueous NaCl solution and dried over Na₂SO₄. The solvent was removed under reduced pressure and the crude residue was purified with column chromatography on silica gel (CH₂Cl₂/MeOH, 9:1) to afford 2 (1340 mg, 85%) as a brown oil.

HPLC (254 nm, System A): t_{R}=13.99 min; HPLC (254 nm, System B): t_{R}=13.31 min; ESI-MS: m/z=345.04 [M+H]⁺

### (1R,3R,5S)-8-Methyl-8-azabicyclo[3.2.1]octan-3-yl (2-bromo-4-fluorophenyl)carbamate (5)

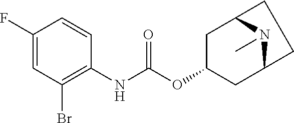 [SMILES: CN1[C@@H]2CC[C@H]1C[C@@H](OC(=O)Nc1ccc(F)cc1Br)C2]

Compound 5 was prepared according to compound 2, using a solution of 2-bromo-4-fluorobenzoic acid (1000 mg, 4.57 mmol) in dry toluene (10 mL), dimethyl formamide (10 μL), SOCl₂ (0.67 mL), NaN₃ (2.5 eq) and tropine (1.5 eq). Purification by column chromatography on silica gel (CH₂Cl₂/MeOH, 9:1) afforded 5 (729 mg, 44%) as a brown oil.

HPLC (254 nm, System C): t_{R}=2.8 min; ESI-MS: m/z=359.00 [M+H]⁺

### Methyl 2-bromo-4-fluorobenzoate (6)

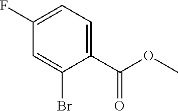

To a solution of 2-bromo-4-fluorobenzoic acid (1000 mg, 4.57 mmol) in MeOH (10 mL) was added concentrated aqueous HCl solution (100 μL) and the mixture was stirred under reflux conditions for 16 h. After removing the organic solvent under reduced pressure, saturated aqueous NaHCO₃ solution was added and the aqueous layer was extracted three times with CHCl₃. The combined organic layer was washed with saturated aqueous NaCl solution and was dried over Na₂SO₄. The organic solvent was removed under reduced pressure to afford 6 (987 mg, 93%) as yellow oil.

HPLC (254 nm, System A): t_{R}=19.45 min; HPLC (254 nm, System B): t_{R}=18.57 min

### Methyl 4-fluoro-2-(thiophen-2-yl)benzoate (7)

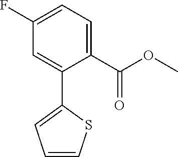 [SMILES: COC(=O)c1ccc(F)cc1-c1cccs1]

Compound 7 was prepared according to general protocol 2 using a solution of 6 (300 mg, 1.29 mmol) in 1,4-dioxane (4 mL), thiophene-2-boronic acid (2 eq), 1′1[bis(diphenylphosphino)ferrocene]dichloropalladium(II) (0.2 eq), Na₂CO₃ (3 eq) and H₂O (1 mL). Purification by column chromatography on silica gel (hexane/ethyl acetate 9:1) afforded 7 (233 mg, 77%) as a dark yellow oil.

HPLC (254 nm, System A): t_{R}=20.19 min; HPLC (254 nm, System B): t_{R}=19.49 min; ESI-MS: m/z=258.85 [M+Na]⁺

### 4-Fluoro-2-(thiophen-2-yl)benzoic acid (3)

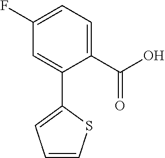 [SMILES: O=C(O)c1ccc(F)cc1-c1cccs1]

To a solution of 7 (200 mg, 847 μmol) in EtOH (4 mL) was added aqueous NaOH solution (2 mL, 1M) and the mixture was stirred under reflux conditions for 16 h. After allowing the reaction to cool to room temperature the organic solvent was removed under reduced pressure. The reaction was acidified with aqueous HCI solution (1M) and the aqueous layer was extracted three times with CHCl₃. The combined organic layer was washed with saturated aqueous NaCl solution and was dried over Na₂SO₄. The organic solvent was removed under reduced pressure to obtain 3 (180 mg, 96%) as a yellow oil.

HPLC (254 nm, System A): t_{R}=19.20 min; HPLC (254 nm, System B): t_{R}=16.99 min; ESI-MS: m/z=244.74 [M+Na]⁺

### 6,7-Dehydroatropine (8)

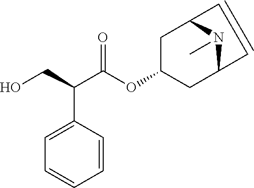 [SMILES: CN1[C@@H]2C=C[C@H]1C[C@@H](OC(=O)[C@H](CO)c1ccccc1)C2]

To a solution of scopolamine hydrobromide trihydrate (500 mg, 1.14 mmol) in dry ethanol (4 mL) was added zinc copper alloy (1-3% copper, 1 g) and the suspension was stirred under reflux conditions for 16 h. After allowing the reaction to cool to room temperature, the organic solvent was removed under reduced pressure. The residue was dissolved in saturated aqueous NaHCO₃ solution and the aqueous layer was extracted three times with CHCl₃. The combined organic layer was washed with saturated aqueous NaCl solution and dried over Na₂SO₄). The organic solvent was removed under reduced pressure to obtain 8 (289 mg, 88%) as colorless oil.

ESI-MS: m/z=287.94 [M+Na]⁺

### 6,7-Dehydrotropine (9)

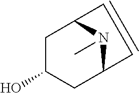

A solution of 8 (329 mg, 1.14 mmol) in aqueous NaOH solution (2M, 4 mL) was stirred for 30 min at room temperature. The aqueous mixture was extracted three times with CH₂Cl₂ and the combined organic layer was washed with saturated aqueous NaCl solution and dried over Na₂SO₄. The organic solvent was removed at atmospheric pressure in a N₂-stream to obtain 9 (116 mg, 73%) as a pale yellow oil.

ESI-MS: m/z=139.89 [M+H]⁺

### (1R,2R,4S,5S,7S)-7-Hydroxy-9,9-dimethyl-3-oxa-9-azatricyclo[3.3.1.0^{2,4}]nonan-9-ium iodide (N-methylscopinium) (10)

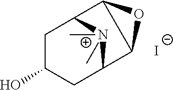

To a solution of scopine (100 mg, 644 μmol) in CH₂Cl₂ (4 mL) was added methyliodide (5 eq) under argon atmosphere and the mixture was stirred for 48 h at room temperature. The solvent was removed under reduced pressure to afford N-methylscopinium iodide (10) (162 mg, 85%) as a white solid.

HPLC (230 nm, System A): t_{R}=3.34 min; HPLC (230 nm, System B): t_{R}=2.95 min; ESI-MS: m/z=169.81 [M]⁺

### 5-Fluoro-[1,1′-biphenyl]-2-amine (11)

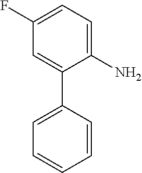 [SMILES: Nc1ccc(F)cc1-c1ccccc1]

Compound 11 was prepared according to the following references: Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

### 2′-Chloro-5-fluoro-[1,1′-biphenyl]-2-amine (12)

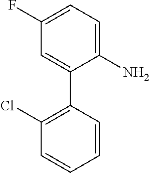 [SMILES: Nc1ccc(F)cc1-c1ccccc1Cl]

Compound 12 was prepared according to the following reference: Regioselective Radical Arylation of Anilines with Arylhydrazines, H. Jasch, J. Scheumann, M. R. Heinrich, J. Org. Chem. 2012, 77, 10699-10706.

4′,5-Difluoro[1,1′biphenyl]-2-amine (13)

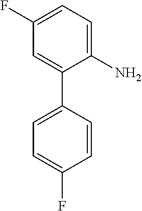 [SMILES: Nc1ccc(F)cc1-c1ccc(F)cc1]

Compound 12 was prepared according to the following references:

Radical arylation of anilines and pyrroles via aryldiazotates, J. Hofmann, E. Gans, T. Clark, M. R. Heinrich, Chem. Eur. J. 2017, 23, 9647-9656

Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

### 4′-Chloro-5-fluoro-[1,1′-biphenyl]-2-amine (14)

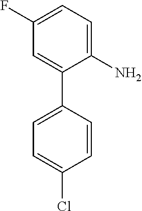 [SMILES: Nc1ccc(F)cc1-c1ccc(Cl)cc1]

Compound 14 was prepared according to the following references:

Radical arylation of anilines and pyrroles via aryldiazotates, J. Hofmann, E. Gans, T. Clark, M. R. Heinrich, Chem. Eur. J. 2017, 23, 9647-9656

Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

Regioselective Radical Arylation of Anilines with Arylhydrazines, H. Jasch, J. Scheumann, M. R. Heinrich, J. Org. Chem. 2012, 77, 10699-10706.

### 4′-Bromo-5-fluoro-[1,1′-biphenyl]-2-amine (15)

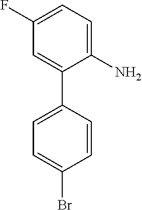 [SMILES: Nc1ccc(F)cc1-c1ccc(Br)cc1]

Compound 15 was prepared according to the following references:

Radical arylation of anilines and pyrroles via aryldiazotates, J. Hofmann, E. Gans, T. Clark, M. R. Heinrich, Chem. Eur. J. 2017, 23, 9647-9656

Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

Regioselective Radical Arylation of Anilines with Arylhydrazines, H. Jasch, J. Scheumann, M. R. Heinrich, J. Org. Chem. 2012, 77, 10699-10706.

### 2′-Amino-5′-fluoro[1,1′-biphenyl]-4-carbonitrile (16)

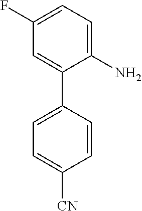 [SMILES: N#Cc1ccc(-c2cc(F)ccc2N)cc1]

Compound 16 was prepared according to the following references:

Oxidative radical arylation of anilines with arylhydrazines and dioxygen from air, J. Hofmann, H. Jasch, M. R. Heinrich, J. Org. Chem. 2014, 79, 2314-2320.

Regioselective Radical Arylation of Anilines with Arylhydrazines, H. Jasch, J. Scheumann, M. R. Heinrich, J. Org. Chem. 2012, 77, 10699-10706.

### 5-Fluoro-4′-methoxy-[1,1′-biphenyl]-2-amine (17)

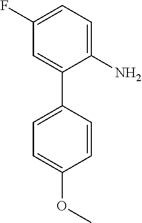 [SMILES: COc1ccc(-c2cc(F)ccc2N)cc1]

Compound 17 was prepared according to the following reference:

Oxidative radical arylation of anilines with arylhydrazines and dioxygen from air, J. Hofmann, H. Jasch, M. R. Heinrich, J. Org. Chem. 2014, 79, 2314-2320.

### 3′,4′-Dichloro-5-fluoro[1,1′-biphenyl]-2-amine (18)

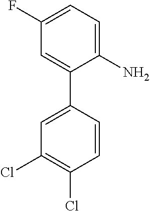 [SMILES: Nc1ccc(F)cc1-c1ccc(Cl)c(Cl)c1]

Compound 18 was prepared according to the following reference:

Oxidative radical arylation of anilines with arylhydrazines and dioxygen from air, J. Hofmann, H. Jasch, M. R. Heinrich, J. Org. Chem. 2014, 79, 2314-2320.

### 3′,4′,5,5′-Tetrafluoro-[1,1′-biphenyl]-2-amine (19)

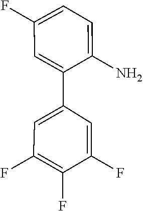 [SMILES: Nc1ccc(F)cc1-c1cc(F)c(F)c(F)c1]

Compound 19 was prepared according to the following reference:

Oxidative radical arylation of anilines with arylhydrazines and dioxygen from air, J. Hofmann, H. Jasch, M. R. Heinrich, J. Org. Chem. 2014, 79, 2314-2320.

### 4,5-Difluoro-(2-phenyl)-phenyl-1-amine (20)

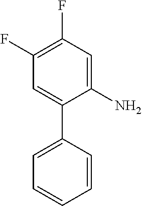 [SMILES: Nc1cc(F)c(F)cc1-c1ccccc1]

Compound 20 was prepared according to the following references:

Radical arylation of anilines and pyrroles via aryldiazotates, J. Hofmann, E. Gans, T. Clark, M. R. Heinrich, Chem. Eur. J. 2017, 23, 9647-9656

Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

Regioselective Radical Arylation of Anilines with Arylhydrazines, H. Jasch, J. Scheumann, M. R. Heinrich, J. Org. Chem. 2012, 77, 10699-10706.

### 3,4,5-Trifluoro-(2-phenyl)-phenyl-1-amine (21)

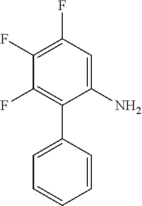 [SMILES: Nc1cc(F)c(F)c(F)c1-c1ccccc1]

Compound 21 was prepared according to the following references:

Radical arylation of anilines and pyrroles via aryldiazotates, J. Hofmann, E. Gans, T. Clark, M. R. Heinrich, Chem. Eur. J. 2017, 23, 9647-9656

Oxidative radical arylation of anilines with arylhydrazines and dioxygen from air, J. Hofmann, H. Jasch, M. R. Heinrich, J. Org. Chem. 2014, 79, 2314-2320.

The Gomberg-Bachmann Reaction for the Arylation of Anilines with Aryl Diazotates, G. Pratsch, T. Wallaschkowski, M. R. Heinrich, Chem. Eur. J. 2012, 18, 11555-11559.

Regioselective Radical Arylation of Anilines with Arylhydrazines, H. Jasch, J. Scheumann, M. R. Heinrich, J. Org. Chem. 2012, 77, 10699-10706.

### tert-Butyl 4-((((5-fluoro-[1,1′-biphenyl]-2-yl)carbamoyl)oxy)methyl)piperidine-1-carboxylate (22)

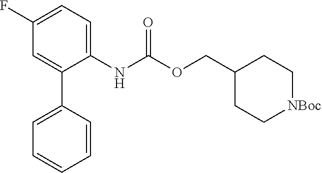 [SMILES: CC(C)(C)OC(=O)N1CCC(COC(=O)Nc2ccc(F)cc2-c2ccccc2)CC1]

Compound 22 was prepared according to general procedure 5 using a solution of intermediate 11. Purification by column chromatography on silica gel afforded 22.

### tert-Butyl 4-((((2′-chloro-5-fluoro-[1,1′-biphenyl]-2-yl)carbamoyl)oxy)methyl)piperidine-1-carboxylate (23)

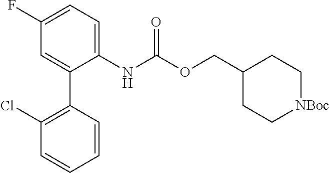 [SMILES: CC(C)(C)OC(=O)N1CCC(COC(=O)Nc2ccc(F)cc2-c2ccccc2Cl)CC1]

Compound 23 was prepared according to general procedure 5 using a solution of intermediate 12. Purification by column chromatography on silica gel afforded 23.

### tert-Butyl 4-((((4′,5-difluoro-[1,1′-biphenyl]-2-yl)carbamoyl)oxy)methyl)piperidine-1-carboxylate (24)

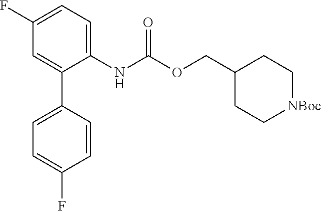 [SMILES: CC(C)(C)OC(=O)N1CCC(COC(=O)Nc2ccc(F)cc2-c2ccc(F)cc2)CC1]

Compound 24 was prepared according to general procedure 5 using a solution of intermediate 13. Purification by column chromatography on silica gel afforded 24.

### tert-Butyl 4-((((4′-chloro-5-fluoro-[1,1′-biphenyl]-2-yl)carbamoyl)oxy)methyl)piperidine-1-carboxylate (25)

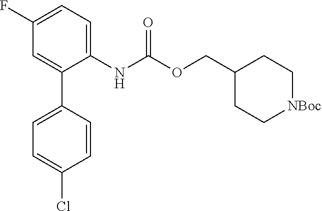 [SMILES: CC(C)(C)OC(=O)N1CCC(COC(=O)Nc2ccc(F)cc2-c2ccc(Cl)cc2)CC1]

Compound 25 was prepared according to general procedure 5 using a solution of intermediate 14. Purification by column chromatography on silica gel afforded 25.

### tert-Butyl 4-((((4′-bromo-5-fluoro-[1,1′-biphenyl]-2-yl)carbamoyl)oxy)methyl)piperidine-1-carboxylate (26)

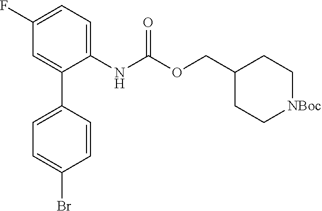 [SMILES: CC(C)(C)OC(=O)N1CCC(COC(=O)Nc2ccc(F)cc2-c2ccc(Br)cc2)CC1]

Compound 26 was prepared according to general procedure 5 using a solution of intermediate 15. Purification by column chromatography on silica gel afforded 26.

### tert-Butyl 4-((((4′-cyano-5-fluoro-[1,1′-biphenyl]-2-yl)carbamoyl)oxy)methyl)piperidine-1-carboxylate (27)

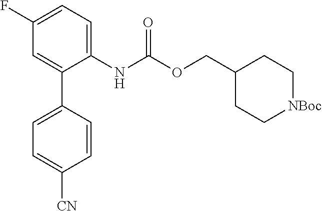 [SMILES: CC(C)(C)OC(=O)N1CCC(COC(=O)Nc2ccc(F)cc2-c2ccc(C#N)cc2)CC1]

Compound 27 was prepared according to general procedure 5 using a solution of intermediate 16. Purification by column chromatography on silica gel afforded 27.

### tert-Butyl 4-((((5-fluoro-4′-methoxy-[1,1′-biphenyl]-2-yl)carbamoyl)oxy)methyl)piperidine-1-carboxylate (28)

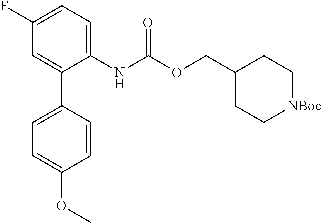 [SMILES: COc1ccc(-c2cc(F)ccc2NC(=O)OCC2CCN(C(=O)OC(C)(C)C)CC2)cc1]

Compound 28 was prepared according to general procedure 5 using a solution of intermediate 17. Purification by column chromatography on silica gel afforded 28.

### tert-Butyl 4-((((3′,4′-dichloro-5-fluoro-[1,1′-biphenyl]-2-yl)carbamoyl)oxy)methyl) piperidine-1-carboxylate (29)

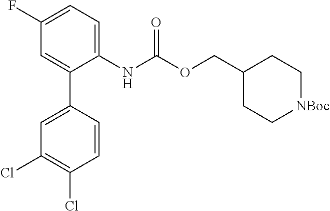 [SMILES: CC(C)(C)OC(=O)N1CCC(COC(=O)Nc2ccc(F)cc2-c2ccc(Cl)c(Cl)c2)CC1]

Compound 29 was prepared according to general procedure 5 using a solution of intermediate 18. Purification by column chromatography on silica gel afforded 29.

### tert-Butyl 4-((((3′,4′,5,5′-tetrafluoro-[1,1′-biphenyl]-2-yl)carbamoyl)oxy)methyl)piperidine-1-carboxylate (30)

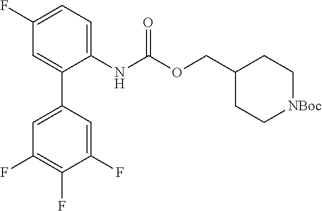 [SMILES: CC(C)(C)OC(=O)N1CCC(COC(=O)Nc2ccc(F)cc2-c2cc(F)c(F)c(F)c2)CC1]

Compound 30 was prepared according to general procedure 5 using a solution of intermediate 19. Purification by column chromatography on silica gel afforded 30.

### 4-Fluoro-2-(thiophen-2-yl)aniline (31)

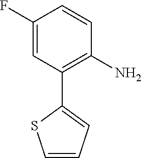 [SMILES: Nc1ccc(F)cc1-c1cccs1]

Intermediate 31 was prepared according to step 1 of general procedure 4 using a solution of 2-bromo-4-fluoroaniline, thiophene-2-boronic acid, 1′1[bis(diphenylphosphino)-ferrocene]dichloropalladium(II) and K₂CO₃ in 1,4-dioxane:H₂O (4:1) and stirring the reaction mixture at 90° C. for 16 h. Purification by column chromatography on silica gel (ethylacetate:hexane) afforded intermediate 31 (424 mg, 32%) as dark red oil.

¹H NMR (400 MHz, Chloroform-d): δ (ppm)=7.36 (dd, J=5.1, 1.2 Hz, 1H), 7.21 (dd, J=3.5, 1.2 Hz, 1H), 7.12 (dd, J=5.2, 3.5 Hz, 1H), 7.01 (dd, J=9.3, 3.0 Hz, 1H), 6.86 (ddd, J=8.8, 8.0, 3.0 Hz, 1H), 6.69 (dd, J=8.8, 4.9 Hz, 1H), 3.85 (s, 2H).

¹³C NMR (101 MHz, Chloroform-d): δ (ppm)=155.95 (d, J=236.4 Hz), 140.07 (d, J=2.1 Hz), 139.93 (d, J=2.0 Hz), 127.64, 126.21, 125.71, 120.92 (d, J=7.7 Hz), 116.90 (d, J=23.0 Hz), 116.80 (d, J=7.9 Hz), 115.50 (d, J=22.3 Hz).

HPLC (254 nm, System A): t_{R}=19.03 min, HPLC (254 nm, System B): t_{R}=14.42 min; ESI-MS: m/z=193.71 [M+H]⁺

### 4-Fluoro-2-(5-methylthiophen-2-yl)aniline (32)

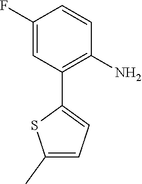

Intermediate 32 was prepared according to step 1 of general procedure 4 using a solution of 2-bromo-4-fluoroaniline, 5-methylthiophene-2-boronic acid pinacol ester, 1′1[bis(diphenylphosphino)ferrocene]dichloropalladium(II) and K₂CO₃ in 1,4-dioxane:H₂O (4:1), stirring the reaction mixture at 90° C. for 16 h. Purification by column chromatography on silica gel (ethylacetate:hexane) afforded intermediate 32 (389 mg, 89%) as red oil.

¹H NMR (400 MHz, Chloroform-d): δ (ppm)=7.03-6.94 (m, 2H), 6.84 (ddd, J=8.8, 8.0, 2.9 Hz, 1H), 6.80-6.72 (m, 1H), 6.69 (dd, J=8.8, 4.9 Hz, 1H), 3.84 (s, 2H), 2.52 (d, J=1.1 Hz, 3H).

¹³C NMR (101 MHz, Chloroform-d): δ (ppm)=156.20 (d, J=236.5 Hz), 140.40, 139.36, 137.39, 126.27 (d, J=1.8 Hz), 125.78, 121.81 (d, J=7.9 Hz), 117.08 (d, J=7.7 Hz), 116.71 (d, J=22.9 Hz), 115.13 (d, J=22.4 Hz), 15.28.

HPLC (254 nm, System A): t_{R}=19.76 min, HPLC (254 nm, System B): t_{R}=16.14 min; ESI-MS: m/z=207.81 [M+H]⁺

### 4-Fluoro-2-(4-methylthiophen-2-yl)aniline (33)

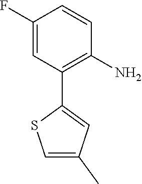

Intermediate 33 was prepared according to step 1 of general procedure 4 using a solution of 2-bromo-4-fluoroaniline, 4-methylthiophene-2-boronic acid pinacol ester, 1′1 [bis(diphenylphosphino)ferrocene]dichloropalladium(II) and K₂CO₃ in 1,4-dioxane:H₂O (4:1) and stirring the reaction mixture at 90° C. for 16 h. Purification by column chromatography on silica gel (ethylacetate:hexane) afforded intermediate 33 (354 mg, 41%) as red oil.

¹H NMR (400 MHz, Chloroform-d): δ (ppm)=7.03 (d, J=1.2 Hz, 2H), 7.00 (dd, J=9.4, 3.0 Hz, 1H), 6.95-6.92 (m, 1H), 6.85 (ddd, J=8.8, 8.0, 2.9 Hz, 1H), 6.69 (dd, J=8.8, 4.9 Hz, 1H), 4.11 (s, 2H), 2.30 (d, J=0.9 Hz, 3H).

HPLC (254 nm, System A): t_{R}=19.80 min, HPLC (254 nm, System B): t_{R}=16.41 min; ESI-MS: m/z=207.82 [M+H]⁺

### 4-Fluoro-2-(3-methylthiophen-2-yl)aniline (34)

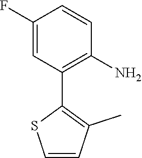

Intermediate 34 was prepared according to step 1 of general procedure 4 using a solution of 2-bromo-4-fluoroaniline, 3-methylthiophene-2-boronic acid pinacol ester, 1′1[bis(diphenylphosphino)ferrocene]dichloropalladium(II) and K₂CO₃ in 1,4-dioxane:H₂O (4:1) and stirring the reaction mixture at 90° C. for 16 h. Purification by column chromatography on silica gel (ethylacetate:hexane) afforded intermediate 34 (375 mg, 86%) as red oil.

¹H NMR (400 MHz, Chloroform-d): δ (ppm)=7.28 (d, J=5.1 Hz, 1H), 6.94 (d, J=5.1 Hz, 1H), 6.93-6.84 (m, 2H), 6.70 (ddd, J=8.6, 4.9, 0.6 Hz, 1H), 3.49 (s, 2H), 2.12 (s, 3H).

HPLC (254 nm, System A): t_{R}=19.63 min, HPLC (254 nm, System B): t_{R}=16.29 min; ESI-MS: m/z=207.83 [M+H]⁺

### 4-Fluoro-2-(thiophen-3-yl)aniline (35)

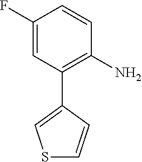 [SMILES: Nc1ccc(F)cc1-c1ccsc1]

Intermediate 35 was prepared according to step 1 of general procedure 4 using a solution of 2-bromo-4-fluoroaniline, thiophene-3-boronic acid, 1′1[bis(diphenylphosphino) ferrocene] dichloropalladium(II) and K₂CO₃ in 1,4-dioxane:H₂O (4:1) and stirring the reaction mixture at 90° C. for 16 h. Purification by column chromatography on silica gel (ethyl acetate:hexane) afforded intermediate 35 (600 mg, 83%) as dark red oil.

¹H NMR (400 MHz, Chloroform-d): δ (ppm)=7.43 (dd, J=4.9, 3.0 Hz, 2H), 7.40 (dd, J=3.0, 1.4 Hz, 2H), 7.28-7.21 (m, 1H), 6.94 (dd, J=9.3, 2.9 Hz, 1H), 6.85 (ddd, J=8.7, 8.1, 3.0 Hz, 1H), 6.69 (dd, J=8.7, 4.9 Hz, 1H), 3.68 (s, 2H).

¹³H NMR (101 MHz, Chloroform-d): δ (ppm)=156.17 (d, J=236.0 Hz), 139.92 (d, J=2.2 Hz), 138.86 (d, J=1.8 Hz), 128.08, 126.37, 123.48 (d, J=7.4 Hz), 123.04, 116.57 (d, J=7.9 Hz), 116.33 (d, J=22.7 Hz), 114.88 (d, J=22.3 Hz).

HPLC (254 nm, System A): t_{R}=17.86 min, HPLC (254 nm, System B): t_{R}=13.90 min; ESI-MS: m/z=193.73 [M+H]⁺

### 2-(Benzo[b]thiophen-2-yl)-4-fluoroaniline (36)

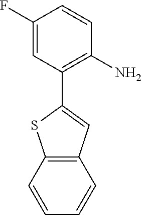 [SMILES: Nc1ccc(F)cc1-c1cc2ccccc2s1]

Intermediate 36 was prepared according to step 1 of general procedure 4 using a solution of 2-bromo-4-fluoroaniline, benzo[b]thien-2-ylboronic acid, [bis(diphenylphosphino)ferrocene]dichloropalladium(Il) and K₂CO₃ in 1,4-dioxane:H₂O (4:1) and stirring the reaction mixture at 90° C. for 16 h. Purification by column chromatography on silica gel (ethylacetate:hexane) afforded intermediate 36 (1080 mg, 84%) as dark red oil.

¹H NMR (400 MHz, Chloroform-d): δ (ppm)=7.89-7.82 (m, 1H), 7.82-7.76 (m, 1H), 7.44 (d, J=0.8 Hz, 1H), 7.41-7.32 (m, 2H), 7.10 (dd, J=9.3, 2.9 Hz, 1H), 6.91 (ddd, J=8.8, 8.0, 3.0 Hz, 1H), 6.73 (dd, J=8.8, 4.8 Hz, 1H), 3.86 (s, 2H).

¹³C NMR (101 MHz, Chloroform-d): δ (ppm)=155.96 (d, J=236.9 Hz), 140.31 (d, J=2.2 Hz), 140.27 (d, J=2.0 Hz), 140.15, 139.73, 124.60, 124.52, 123.60, 122.95, 122.14, 120.70 (d, J=7.8 Hz), 117.04 (d, J=7.8 Hz), 117.02 (d, J=23.1 Hz), 116.13 (d, J=22.4 Hz).

HPLC (254 nm, System A): t_{R}=20.97 min, HPLC (254 nm, System B): t_{R}=19.32 min; ESI-MS: m/z=243.77 [M+H]⁺

### 2-(Benzo[b]thiophen-3-yl)-4-fluoroaniline (37)

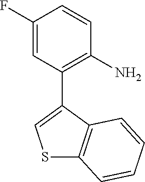 [SMILES: Nc1ccc(F)cc1-c1csc2ccccc12]

Intermediate 37 was prepared according to step 1 of general procedure 4 using a solution of 2-bromo-4-fluoroaniline, benzo[b]thien-3-yiboronic acid, 1′1[bis(diphenylphosphino)ferrocene]dichloropalladium(II) and K₂CO₃ in 1,4-dioxane:H₂O (4:1) and stirring the reaction mixture at 90° C. for 16 h. Purification by column chromatography on silica gel (ethylacetate:hexane) afforded intermediate 37 (242 mg, 19%) as dark red oil.

¹H NMR (400 MHz, Chloroform-d): δ (ppm)=7.96-7.90 (m, 1H), 7.65-7.59 (m, 1H), 7.46 (s, 1H), 7.42-7.35 (m, 2H), 6.99-6.91 (m, 2H), 6.76 (dd, J=9.6, 4.9 Hz, 1H), 3.23 (s, 2H).

¹³C NMR (101 MHz, Chloroform-d): δ (ppm)=155.93 (d, J=236.6 Hz), 140.81, 140.33, 137.80, 133.83, 125.25, 124.75, 124.48, 123.18, 122.89, 122.01 (d, J=7.8 Hz), 117.31 (d, J=22.3 Hz), 116.42 (d, J=7.8 Hz), 115.62 (d, J=22.1 Hz).

HPLC (254 nm, System A): t_{R}=20.50 min, HPLC (254 nm, System B): t_{R}=17.43 min; ESI-MS: m/z=243.77 [M+H]⁺

### 2-(Thiophen-2-yl)aniline (38)

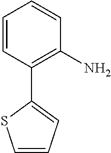 [SMILES: Nc1ccccc1-c1cccs1]

Intermediate 38 was prepared according to step 1 of general procedure 4 using a solution of 2-bromoaniline, thiophene-2-boronic acid, 1′1[bis(diphenylphosphino)ferrocene]dichloropalladium(II) and K₂CO₃ in 1,4-dioxane:H₂O (4:1) and stirring the reaction mixture at 90° C. for 16 h. Purification by column chromatography on silica gel (ethylacetate:hexane) afforded intermediate 38 (343 mg, 42%) as red oil.

¹H NMR (400 MHz, Chloroform-d): δ (ppm)=7.34 (dd, J=5.2, 1.2 Hz, 1H), 7.29-7.26 (m, 1H), 7.20 (dd, J=3.5, 1.2 Hz, 1H), 7.17-7.10 (m, 2H), 6.84-6.73 (m, 2H), 3.97 (s, 2H).

¹³C NMR (101 MHz, Chloroform-d): δ (ppm)=143.94, 141.02, 130.96, 129.00, 127.52, 125.78, 125.21, 119.95, 118.55, 115.85.

HPLC (254 nm, System A): t_{R}=18.42 min, HPLC (254 nm, System B): t_{R}=14.61 min; ESI-MS: m/z=175.80 [M+H]⁺

### (1R,2R,4S,5S,7S)-9-Methyl-3-oxa-9-azatricyclo[3.3.1.0^{2,4}]nonan-7-yl carbonochloridate (40)

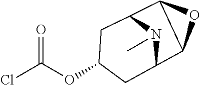

In a flame dried flask containing a stirred suspension of scopine (0.24 mmol, 1.0 eq.) in dry ACN (0.8 mL), triphosgene (0.24 mmol, 1.0 eq.) was added portion-wise at 0° C. The reaction mixture was stirred at this temperature for 5 min and, then, at room temperature for 36 h. The solvent was removed under reduced pressure and the residue was triturated with dry Et₂O. The crude product containing compound 40 as hydrochloride was used for the next step.

### SYNTHESIS AND CHARACTERIZATION OF INVENTIVE EXAMPLES

### Piperidin-4-ylmethyl (5-fluoro-[1,1′-biphenyl]-2-yl)carbamate (EXP 1)

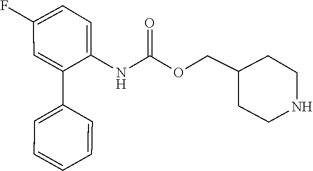 [SMILES: O=C(Nc1ccc(F)cc1-c1ccccc1)OCC1CCNCC1]

Compound EXP 1 was prepared according to general procedure 5 using intermediate 22. Without any further purification the trifluoroacetate salt of EXP 1 was afforded as dark brown oil (12 mg, 100%).

¹H-NMR (600 MHz, CD₃OD): δ (ppm)=1.38 (dt, J=3.9 Hz, J=14.9 Hz, 2H), 1.76-1.92 (m, 3H), 2.94 (t, J=12.1 Hz, 2H), 3.37 (d, J=12.7 Hz, 2H), 3.87-3.96 (m, 2H), 7.05-7.13 (m, 2H), 7.36-7.40 (m, 3H), 7.42-7.45 (m, 2H), 7.46-7.55 (m, 1H).

¹³C-NMR (151 MHz, CD₃OD): δ (ppm)=26.5 (2×CH₂), 34.7 (CH), 44.7 (2×CH₂), 69.0 (CH₂), 115.6 (d, J_{CF}=22.5 Hz, CH), 117.9 (d, J_{CF}=23.1 Hz, CH), 128.9 (d, J_{CF}=8.1 Hz, CH), 129.7 (3×CH), 129.9 (d, J_{CF}=8.3 Hz, C_{q}), 130.0 (2×CH), 131.9 (d, J_{CF}=2.9 Hz, C_{q}), 132.0 (C_{q}), 139.6 (d, J_{CF}=245.3 Hz, C_{q}), 156.9 (C_{q}).

EI-MS: m/z (%): 213 (26), 187 (42), 186 (22), 185 (24), 98 (100), 96 (20), 69 (35), 56 (48), 45 (18), 42 (16), 41 (24), 30 (16).

### Piperidin-4-ylmethyl (2′-chloro-5-fluoro-[1,1′-biphenyl]-2-yl)carbamate (EXP 2)

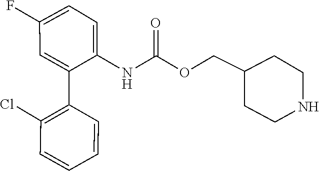 [SMILES: O=C(Nc1ccc(F)cc1-c1ccccc1Cl)OCC1CCNCC1]

Compound EXP 2 was prepared according to general procedure 5 using intermediate 23. Without any further purification the trifluoroacetate salt of EXP 2 was afforded as dark brown oil (25 mg, 92%).

¹H-NMR (600 MHz, CD₃OD): δ (ppm)=1.27 (dt, J=4.0 Hz, J=15.6 Hz, 2H), 1.73 (d, J=14.2 Hz, 2H), 1.75-1.85 (m, 1H), 2.83 (dt, J=2.8 Hz, J=12.9 Hz, 2H), 3.26 (d, J=13.0 Hz, 2H), 3.77-3.83 (m, 2H), 6.87 (dd, J=2.9 Hz, J=8.9 Hz, 1H), 7.05 (ddd, J=3.0 Hz, J=8.2 Hz, J=8.9 Hz, 1H), 7.18-7.31 (m, 3H), 7.37-7.40 (m, 1H), 7.43 (s, 1H).

¹³C-NMR (151 MHz, CD₃OD): δ (ppm)=26.5 (2×CH₂), 34.8 (CH), 44.7 (2×CH₂), 69.1 (CH₂), 116.4 (d, J_{CF}=22.5 Hz, CH), 118.4 (CH), 118.2 (d, J_{CF}=22.5 Hz, CH), 128.2 (CH), 130.8 (2×CH), 132.9 (d, C_{q}), 133.0 (CH), 134.4 (C_{q}), 137.9 (d, C_{q}), 156.6 (C_{q}).

EI-MS: m/z (%): 362 (26), 221 (41), 186 (48), 185 (50), 184 (53), 114 (39), 98 (100), 97 (25), 96 (32), 82 (22), 69 (71), 56 (67), 51 (21), 42 (25), 41 (36).

### Piperidin-4-ylmethyl (4′,5-difluoro-[1,1-biphenyl]-2-yl)carbamate (EXP 3)

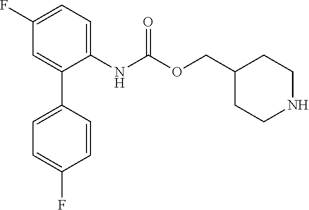 [SMILES: O=C(Nc1ccc(F)cc1-c1ccc(F)cc1)OCC1CCNCC1]

Compound EXP 3 was prepared according to general procedure 5 using intermediate 24. Purification by column chromatography on silica gel afforded EXP 3 was afforded as dark brown oil.

¹H-NMR (600 MHz, CD₃OD): δ (ppm)=1.39 (dt, J=4.5 Hz, J=10.9 Hz, 2H), 1.79-1.90 (m, 3H), 2.95 (t, J=11.7 Hz, 2H), 3.38 (d, J=12.8 Hz, 2H), 3.91 (d, J=6.0 Hz, 2H), 7.04-7.13 (m, 2H), 7.17 (t, J=8.9 Hz, 2H), 7.37-7.49 (m, 3H).

¹³C-NMR (151 MHz, CD₃OD): δ (ppm)=26.5 (2×CH₂), 34.8 (CH), 44.7 (2×CH₂), 69.1 (CH₂), 115.8 (d, J_{CF}=22.5 Hz, CH), 116.4 (d, J_{CF}=21.7 Hz, 2×CH), 117.9 (d, J_{CF}=23.1 Hz, CH), 131.9 (CH),132.0 (d, J_{CF}=8.2 Hz, 2×CH), 135.9 (m, C_{q}), 156.9 (C_{q}), 161.2 (C_{q}), 162.9 (C_{q}), 163.9 (d, J_{CF}×239.6 Hz, C_{q}).

### Piperidin-4-ylmethyl (4′-chloro-5-fluoro-[1,1′-biphenyl]-2-yl)carbamate (EXP 4)

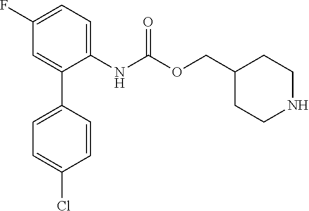 [SMILES: O=C(Nc1ccc(F)cc1-c1ccc(Cl)cc1)OCC1CCNCC1]

Compound EXP 4 was prepared according to general procedure 5 using intermediate 25. Purification by column chromatography on silica gel afforded EXP 4 as dark brown oil.

¹H-NMR (600 MHz, CD₃OD): δ (ppm)=1.37 (dt, J=4.0 Hz, J=11.5 Hz, 2H), 1.79-1.90 (m, 3H), 2.95 (t, J=12.2 Hz, 2H), 3.38 (d, J=12.6 Hz, 2H), 3.87-3.94 (m, 2H), 7.09 (dd, J=2.9 Hz, J=9.1 Hz, 1H), 7.12 (ddd, J=3.0 Hz, J=8.2 Hz, J=8.7 Hz, 1H), 7.38 (d, J=8.4 Hz, 2H), 7.42-7.45 (m, 1H) 7.44 (d, J=8.6 Hz, 2H).

¹³C-NMR (151 MHz, CD₃OD): δ (ppm)=26.5 (2×CH₂), 34.9 (CH), 44.8 (2×CH₂), 69.1 (CH₂), 116.0 (d, J_{CF}=22.5 Hz, CH), 117.8 (d, J_{CF}=23.3 Hz, CH), 129.7 (2×CH), 131.7 (2×CH), 131.9 (d, CH), 134.8 (C_{q}), 138.5 (d, J_{CF}=1.6 Hz, C_{q}), 140.2 (C_{q}), 156.9 (C_{q}), 162.2 (d, J_{CF}=241.0 Hz, C_{q}).

### Piperidin-4-ylmethyl (4′-bromo-5-fluoro-[1,1′-biphenyl]-2-yl)carbamate (EXP 5)

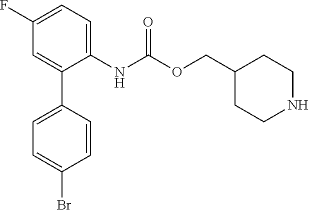 [SMILES: O=C(Nc1ccc(F)cc1-c1ccc(Br)cc1)OCC1CCNCC1]

Compound EXP 5 was prepared according to general procedure 5 using intermediate 26. Without any further purification the trifluoroacetate salt of EXP 5 was afforded as brown oil (7 mg, 99%).

¹H-NMR (600 MHz, CD₃OD): δ (ppm)=1.32-1.43 (m, 2H), 1.76-1.94 (m, 3H), 2.96 (t, J=12.7 Hz, 2H), 3.39 (d, J=12.7 Hz, 2H), 3.86-3.98 (m, 2H), 7.07-7.15 (m, 3H), 7.28-7.35 (m, 2H), 7.57-7.61 (m, 2H).

¹³C-NMR (151 MHz, CD₃OD): δ (ppm)=26.5 (2×CH₂), 34.8 (CH), 44.8 (2×CH₂), 69.0 (CH₂), 116.1 (d, J_{CF}=22.5 Hz, CH), 117.6 (d, J_{CF}=23.3 Hz, CH), 122.8 (C_{q}), 132.0 (2×CH), 132.7 (2×CH), 139.0 (C_{q}), 156.9 (C_{q}).

EI-MS: m/z (%): 406 (36), 391 (21), 300 (14), 291 (43), 277 (42), 248 (20), 153 (53), 114 (39), 98 (100), 97 (25), 96 (32), 82 (20), 69 (51), 56 (43), 51 (14), 42 (35), 41 (36), 30 (15).

### Piperidin-4-ylmethyl (4′-cyano-5-fluoro-[1,1′-biphenyl]-2-yl)carbamate (EXP 6)

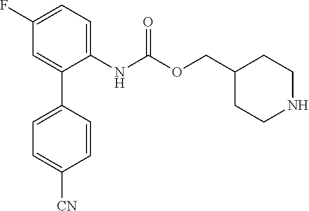 [SMILES: N#Cc1ccc(-c2cc(F)ccc2NC(=O)OCC2CCNCC2)cc1]

Compound EXP 6 was prepared according to general procedure 5 using intermediate 27. Without any further purification the trifluoroacetate salt of EXP 6 was afforded as brown oil (99 mg, 97%).

¹H-NMR (600 MHz, CD₃OD): δ (ppm)=1.32-1.44 (m, 2H), 1.78-1.92 (m, 3H), 2.96 (dt, J=2.0 Hz, J=12.9 Hz, 2H), 3.39 (d, J=12.7 Hz, 2H), 3.86 (d, J=6.2 Hz, 2H), 7.13-7.21 (m, 2H), 7.45 (dd, J=5.3 Hz, J=8.6 Hz, 1H), 7.58 (d, J=8.6 Hz, 2H), 7.80 (d, J=8.6 Hz, 2H).

¹³C-NMR (151 MHz, CD₃OD): δ (ppm)=26.6 (2×CH₂), 34.8 (CH), 44.8 (2×CH₂), 69.2 (CH₂), 112.5 (C_{q}), 116.8 (d, J_{CF}=22.5 Hz, CH) 117.8 (d, J_{CF}=23.6 Hz, CH), 119.7 (C_{q}), 131.1 (2×CH), 131.3 (d, J_{CF}=8.3 Hz, CH), 132.0 (C_{q}), 133.4 (2×CH), 133.6 (C_{q}), 144.9 (d, J=2.0 Hz, C_{q}), 148.2 (C_{q}), 156.9 (C_{q}).

EI-MS: m/z (%): 354 (43), 353 (21), 238 (100), 211 (36), 210 (45), 157 (21), 114 (43), 105 (34), 97 (21), 96 (31), 84 (21), 69 (26), 56 (41), 44 (21), 42 (51), 41 (38), 30 (24).

### Piperidin-4-ylmethyl (5-fluoro-4′-methoxy-[1,1′-biphenyl]-2-yl)carbamate (EXP 7)

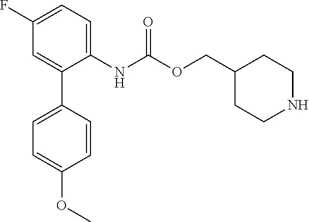 [SMILES: COc1ccc(-c2cc(F)ccc2NC(=O)OCC2CCNCC2)cc1]

Compound EXP 7 was prepared according to general procedure 5 using intermediate 28. Without any further purification the trifluoroacetate salt of EXP 7 was afforded as brown oil (10 mg, 92%).

¹H-NMR (600 MHz, CD₃OD): δ (ppm)=1.32-1.41 (m, 2H), 1.78-1.92 (m, 3H), 2.94 (t, J=12.3 Hz, 2H), 3.37 (d, J=12.9 Hz, 2H), 3.83 (s, 3H), 3.88-3.96 (m, 2H), 6.99 (d, J=8.6 Hz, 2H), 7.02-7.08 (m, 3H), 7.31 (d, J=8.6 Hz, 2H).

¹³C-NMR (151 MHz, CD₃OD): δ (ppm)=26.6 (2×CH₂), 34.9 (CH), 44.8 (2×CH₂), 55.9 (CH₃), 69.1 (CH₂), 115.1 (2×CH), 115.2 (d, J_{CF}=22.7 Hz, CH), 115.3 (C_{q}), 117.7 (d, J_{CF}=22.9 Hz, CH), 131.2 (2×CH), 131.3 (C_{q}), 156.9 (C_{q}), 160.9 (C_{q}).

EI-MS: m/z (%): 358 (23), 244 (46), 216 (52), 201 (21), 188 (41), 182 (33), 151 (11), 98 (100), 85 (16), 56 (24), 42 (20), 41 (18), 15 (30).

### Piperidin-4-ylmethyl (3′,4′-dichloro-5-fluoro-[1,1′-biphenyl]-2-yl)carbamate (EXP 8)

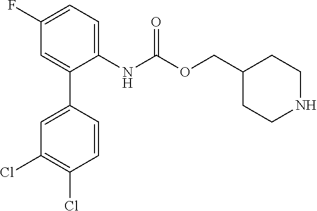 [SMILES: O=C(Nc1ccc(F)cc1-c1ccc(Cl)c(Cl)c1)OCC1CCNCC1]

Compound EXP 8 was prepared according to general procedure 5 using intermediate 29. Without any further purification the trifluoroacetate salt of EXP 8 was afforded as brown oil.

¹H-NMR (600 MHz, CD₃OD): δ (ppm)=1.39 (dt, J=4.5 Hz, J=11.5 Hz, 2H), 1.80-1.92 (m, 3H), 2.95 (t, J=11.9 Hz, 2H), 3.39 (d, J=12.8 Hz, 2H), 3.89-3.93 (m, 2H), 7.12 (m, 1H), 7.15 (ddd, J=3.0 Hz, J=8.1 Hz, J=8.7 Hz, 1H), 7.32 (m, 1H), 7.39-7.44 (m, 1H), 7.57 (d, J=2.0 Hz, 1H), 7.59 (d, J=8.3 Hz, 1H).

¹³C-NMR (151 MHz, CD₃OD): δ (ppm)=26.5 (2×CH₂), 34.9 (CH), 44.8 (2×CH₂), 69.1 (CH₂), 116.5 (d, J_{CF}=22.5 Hz, CH), 117.8 (d, J_{CF}=23.6 Hz, CH), 130.0 (CH), 131.7 (CH), 131.9 (CH), 132.7 (CH), 133.2 (C_{q}), 139.2 (C_{q}), 140.2 (C_{q}), 156.3 (d, J_{CF}=241.0 Hz, C_{q}), 161.5 (C_{q}).

### Piperidin-4-ylmethyl (3′,4′,5,5′-tetrafluoro-[1,1′-biphenyl]-2-yl)carbamate (EXP 9)

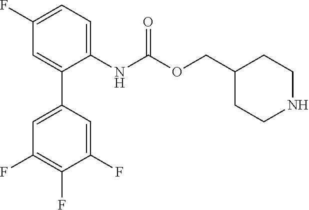 [SMILES: O=C(Nc1ccc(F)cc1-c1cc(F)c(F)c(F)c1)OCC1CCNCC1]

Compound EXP 9 was prepared according to general procedure 5 using intermediate 30. Without any further purification the trifluoroacetate salt of EXP 9 was afforded as black oil (9 mg, 100%).

¹H-NMR (600 MHz, CD₃OD): δ (ppm)=1.37-1.48 (m, 2H), 1.86-1.98 (m, 3H), 2.97 (t, J=11.9 Hz, 2H), 3.40 (d, J=12.8 Hz, 2H), 3.95 (d, J=5.9 Hz, 2H), 7.13-7.20 (m, 3H), 7.27-7.31 (m, 1H), 7.41-7.44 (m, 1H).

¹³C-NMR (151 MHz, CD₃OD): δ (ppm)=26.6 (2×CH₂), 34.9 (CH), 44.8 (2×CH₂), 69.2 (CH₂), 114.6 (d, J_{CF}=4.6 Hz, C_{q}), 114.6 (dd, J_{CF}=4.6 Hz, 17.3 Hz, 2×CH), 116.8 (d, J_{CF}=22.5 Hz, CH), 118.1 (d, J_{CF}=23.7 Hz, CH), 126.0 (d, J_{CF}=9.3 Hz, CH), 132.0 (d, J_{CF}=3.2 Hz, C_{q}).

EI-MS: m/z (%): 382 (35), 268 (33), 367 (14), 241 (48), 225 (45), 206 (18). 185 (35), 155 (50), 153 (34), 98 (100), 96 (24), 69 (15), 56 (60), 45 (41), 42 (22), 41 (36), 31 (43).

### (1S,3R,4S)-Quinuclidin-3-yl(S)-2-(5-fluoro-[1,1′-biphenyl]-2-yl)-2-hydroxyacetate (EXP 10)

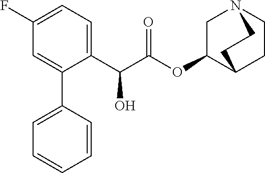 [SMILES: O=C(O[C@H]1C[N@]2CC[C@H]1CC2)[C@@H](O)c1ccc(F)cc1-c1ccccc1]

The diasteromeric mixture of EXP 10 and EXP 11 was prepared according to general procedure 1. The diastereomers were separated by preparative TLC (CH₂Cl₂/MeOH/NH₃ aqu. 25%, 20:1:0.02). The ratio of the diastereomers was determined by NMR. EXP 10 contains <1% of EXP 11.

¹H-NMR (600 MHz, CDCl₃): δ (ppm)=7.47-7.38 (m, 6H), 7.12-7.08 (m, 1H), 7.03 (dd, J=9.4, 2.7 Hz, 1H), 5.25 (s, 1H), 4.85-4.81 (m, 1H), 3.17 (ddd, J=14.8, 8.2, 2.2 Hz, 1H), 2.83-2.69 (m, 3H), 2.59-2.53 (m, 1H), 2.48-2.43 (m, 1H), 2.00-1.96 (m, 1H), 1.71-1.65 (1H), 1.56-1.48 (2H), 1.38-1.28 (1H) (3×m).

HPLC (254 nm, System A): t_{R}=15.8 min, (254 nm, System D): t_{R}=13.7 min; HR ESI-MS: calcd 356.16565, found 356.16590 [M]⁺

### (1S,3R,4S)-Quinuclidin-3-yl (R)-2-(5-fluoro-[1,1′-biphenyl]-2-yl)-2-hydroxyacetate (EXP 11)

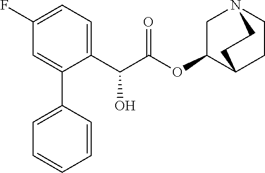 [SMILES: O=C(O[C@H]1C[N@]2CC[C@H]1CC2)[C@H](O)c1ccc(F)cc1-c1ccccc1]

Compound EXP 11 was separated as described for EXP 10. The ratio of the diastereomers was determined by NMR. EXP 11 contains 4% of EXP 10.

¹H-NMR (151 MHz, CDCl₃): δ (ppm)=7.48-7.40 (m, 5H), 7.38 (dd, J=8.7, 5.7 Hz, 1H), 7.10-7.05 (m, 1H), 7.04-7.00 (m, 1H), 5.25 (s, 1H), 4.86-4.82 (m, 1H), 3.49 (s, 1H), 3.18 (ddd, J=14.8, 8.4, 2.3 Hz, 1H), 2.81-2.66 (m, 4H), 2.66-2.61 (m, 1H), 1.91-1.86 (m, 1H), 1.66-1.60 (2H), 1.55-1.46 (1H), 1.24-1.15 (1H) (3m).

HPLC: (254 nm, System A): t_{R}=16.1 min, (254 nm, System D): t_{R}=13.9 min; HR ESI-MS: calcd 356.16565, found 356.16567 [M]⁺

### (1S,3R,4S)-3-(((5-Fluoro-[1,1′-biphenyl]-2-yl)carbamoyl)oxy)-1-methylquinuclidin-1-ium trifluoroacetate (EXP 12)

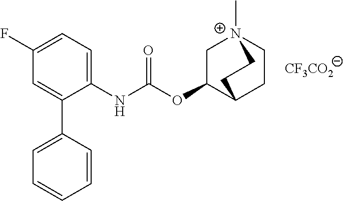

Intermediate compound (1S,3R,4S)-Quinuclidin-3-yl (5-fluoro-[1,1′-biphenyl]-2-yl)carbamate was prepared according to general procedure 6 using intermediate 11. Purification by column chromatography on silica gel afforded (1S,3R,4S)-Quinuclidin-3-yl (5-fluoro-[1,1′-biphenyl]-2-yl)carbamate (206 mg, 60%) as a fawn oil. Compound EXP 12 was then prepared according to general procedure 6 using the (1S,3R,4S)-Quinuclidin-3-yl (5-fluoro-[1,1′-biphenyl]-2-yl)carbamate. Purification by preparative HPLC afforded EXP 12.

¹H-NMR (600 MHz, CDCl₃): δ (ppm)=1.83-1.98 (m, 2H), 2.02-2.10 (m, 1H), 2.12-2.21 (m, 1H), 2.30 (m, 1H), 2.95 (s, 3H), 3.12-3.25 (m, 1H), 3.30-3.36 (m, 1H), 3.41-3.51 (m, 2H), 3.71-3.82 (m, 1H), 4.88-5.00 (m, 1H), 7.05-7.12 (m, 2H), 7.34-7.43 (m, 3H), 7.45-7.48 (m, 2H), 7.50 (bs, 1H).

¹³C-NMR (151 MHz, CDCl₃): δ (ppm)=19.3, 22.1, 25.2, 52.3, 57.1, 58.0, 64.2, 69.3, 115.5 (d, J_{CF}=22.0 Hz, CH), 117.9 (d, J_{CF}=22.8 Hz, CH), 129.0 (d, CH), 129.7 (2×CH), 130.0 (2×CH), 131.6 (d, J_{CF}=2.9 Hz, C_{q}), 139.7 (C_{q}).

HR EI-MS: calculated for C₂₁H₂₄FN₂O₂ [M⁺]: 355.1809, found: 355.1805.

### 1-(5-Fluoro-[1,1′-biphenyl]-2-yl)-3-((1S,3R,4S)-quinuclidin-3-yl)urea (EXP 13)

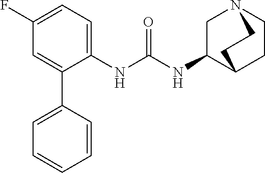 [SMILES: O=C(Nc1ccc(F)cc1-c1ccccc1)N[C@H]1C[N@]2CC[C@H]1CC2]

Compound EXP 13 was prepared according to general procedure 6 using intermediate 11 and (R)-quinuclidin-3-amine. Purification by column chromatography on silica gel (CH₂Cl₂/CH₃OH=15:1) afforded EXP 13 (80 mg, 46%) as fawn oil.

¹H-NMR (600 MHz, CDCl₃, TFA Salz): δ (ppm)=1.81-1.89 (m, 1H), 1.93-2.04 (m, 3H), 2.06-2.10 (m, 1H), 2.93 (ddd, J=2.5 Hz, J=5.1 Hz, J=13.4 Hz, 1H), 3.21-3.33 (m, 4H), 3.66 (ddd, J=2.5 Hz, J=9.6 Hz, J=13.3 Hz, 1H), 4.03-4.08 (m, 1H), 7.01 (dd, J=2.9 Hz, J=9.2 Hz, 1H), 7.07 (ddd, J=3.0 Hz, 8.2 Hz, 8.9 Hz, 1H), 7.35-7.42 (m, 3H), 7.44-7.48 (m, 2H), 7.64 (dd, J=5.3 Hz, J=8.9 Hz, 1H).

¹³C-NMR (91 MHz, CDCl₃): δ (ppm)=18.3, 22.9, 25.9, 46.1, 47.0, 47.6, 54.9, 115.5 (d, J_{CF}=22.3 Hz, CH), 117.7 (d, J_{CF}=23.0 Hz, CH), 128.0 (d, J_{CF}=8.4 Hz, CH), 129.1 (CH), 129.9 (2×CH), 130.0 (2×CH), 132.7 (d, J_{CF}=2.9 Hz, C_{q}), 139.1 (d, J=7.8 Hz, C_{q}), 139.6 (d, J_{CF}=1.6 Hz, C_{q}), 158.3 (C_{q}), 161.2 (d, J_{CF}=243.2 Hz, C_{q}).

HR EI-MS: calculated for C₂₀H₂₃FN₃O [M⁺]: 340.1820, found: 340.1822

### (1S,3R,4S)-3-(3-(5-Fluoro-[1,1′-biphenyl]-2-yl)ureido)-1-methylquinuclidin-1-ium trifluoroacetate (EXP 14)

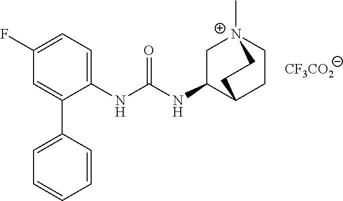

Compound EXP 14 was prepared according to general procedure 6 using EXP 13. Purification by preparative HPLC afforded EXP 14 as fawn oil.

¹H-NMR (600 MHz, CDCl₃, TFA salt): δ (ppm)=1.88-1.98 (m, 1H), 2.00-2.15 (m, 4H), 2.98 (s, 3H), 3.12 (ddd, J=2.9 Hz, J=4.9 Hz, J=13.1 Hz, 1H), 3.35-3.50 (m, 4H), 3.78 (ddd, J=2.5 Hz, J=9.6 Hz, J=13.3 Hz, 1H), 4.07-4.13 (m, 1H), 7.01 (dd, J=2.9 Hz, J=9.2 Hz, 1H), 7.07 (ddd, J=3.0 Hz, J=8.2 Hz, J=8.9 Hz, 1H), 7.36-7.42 (m, 3H), 7.44-7.48 (m, 2H), 7.65 (dd, J=5.3 Hz, J=8.9 Hz, 1H).

¹³C-NMR (91 MHz, CDCl₃): δ (ppm)=19.8, 23.9, 25.8, 47.1, 52.4, 57.3, 57.9, 65.0, 115.5 (d, J_{CF}=22.3 Hz, CH), 117.7 (d, J_{CF}=23.0 Hz, CH), 127.9 (d, J_{CF}=8.4 Hz, CH), 129.1 (CH), 129.9 (2×CH), 130.2 (2×CH), 132.6 (d, J_{CF}=2.9 Hz, C_{q}), 139.1 (d, J_{CF}=7.8 Hz, C_{q}), 139.6 (d, J_{CF}=1.6 Hz, C_{q}), 158.3 (C_{q}), 161.1 (d, J_{CF}=243.1 Hz, C_{q}).

HR EI-MS: calculated for C₂₁H₂₅FN₃O [M⁺]: 354.1974, found: 354.1976

### (1S,3R,4S)-Quinuclidin-3-yl(4,5-difluoro-[2-phenyl]-phen-1-yl)carbamate (EXP 15)

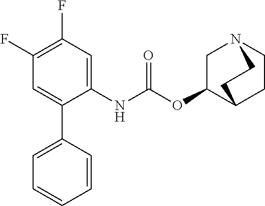 [SMILES: O=C(Nc1cc(F)c(F)cc1-c1ccccc1)O[C@H]1C[N@]2CC[C@H]1CC2]

Compound EXP 15 was prepared according to general procedure 6 using intermediate 20. Purification by column chromatography on silica gel afforded EXP 15 (12 mg, 20%) as fawn oil.

¹H-NMR (600 MHz, CDCl₃): δ (ppm)=1.43-1.48 (m, 1H), 1.60-1.65 (m, 1H), 1.74-1.80 (m, 2H), 2.10-2.13 (m, 1H), 2.75-2.91 (m, 5H), 3.25-3.35 (m, 1H), 4.82-4.84 (m, 1H), 6.59 (s, 1H), 7.03 (dd, J=8.5 Hz, J=10.5 Hz, 1H), 7.30-7.35 (m, 2H), 7.42-7.48 (m, 1H), 7.47-7.54 (m, 2H), 8.01 (bs, 1H).

¹³C-NMR (91 MHz, CDCl₃): δ (ppm)=17.0, 20.6, 24.3, 45.4, 46.2, 53.0, 67.9, 118.6 (m, CH), 118.7 (t, J_{CF}=5.6 Hz CH), 128.8 (CH), 129.0 (2×CH), 129.4 (2×CH), 129.5 (4×CH), 135.9 (C_{q}), 150.8 (d, J=12.9 Hz, C_{q}).

### (1S,3R,4S)-Quinuclidin-3-yl(3,4,5-trifluoro-[2-phenyl]-phen-1-yl)carbamate (EXP 16)

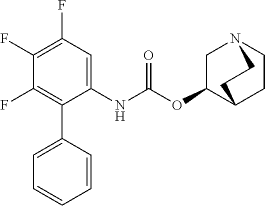 [SMILES: O=C(Nc1cc(F)c(F)c(F)c1-c1ccccc1)O[C@H]1C[N@]2CC[C@H]1CC2]

Compound EXP 16 was prepared according to general procedure 6 using intermediate 21. Purification by column chromatography on silica gel afforded EXP 16 (30 mg, 46%) as fawn oil.

¹H-NMR (600 MHz, CDCl₃): δ (ppm)=1.32-1.40 (m, 1H), 1.52-1.60 (m, 1H), 1.62-1.74 (m, 2H), 2.00-2.04 (m, 1H), 2.63-2.89 (m, 5H), 3.23 (dd, J=8.5 Hz, J=14.6 Hz, 1H), 4.76 (dt, J=2.8 Hz, 7.0 Hz 1H), 6.47 (s, 1H), 7.32 (d, J=8.3 Hz, 2H), 7.48-7.53 (m, 1H), 7.53-7.58 (m, 2H) 7.92 (bs, 1H).

¹³C-NMR (151 MHz, CDCl₃): δ (ppm)=19.2, 24.4, 25.2, 46.5, 47.3, 55.1, 72.7, 129.1 (CH), 129.3 (CH), 129.5 (2×CH), 130.2 (2×CH), 152.8 (C_{q}).

### (1S,3R,4S)-Quinuclidin-3-yl(3′,4′-dichloro-5-fluoro-[1,1′-biphenyl]-2-yl)carbamate (EXP 17)

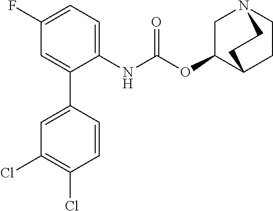 [SMILES: O=C(Nc1ccc(F)cc1-c1ccc(Cl)c(Cl)c1)O[C@H]1C[N@]2CC[C@H]1CC2]

Compound EXP 17 was prepared according to general procedure 6 using intermediate 18. Purification by column chromatography on silica gel afforded EXP 17.

¹H-NMR (600 MHz, CDCl₃): δ (ppm)=1.35-1.48 (m, 1H), 1.53-1.64 (m, 1H), 1.66-1.83 (m, 2H), 2.04-2.10 (m, 1H), 2.66-2.94 (m, 5H), 3.28 (dd, J=8.4 Hz, J=14.6 Hz, 1H), 4.80 (bs, 1H), 6.35 (bs, 1H), 6.94 (dd, J=2.9 Hz, J=8.7 Hz, 1H), 7.09 (ddd, J=3.0 Hz, J=7.9 Hz, J=9.0 Hz, 1H), 7.21 (dd, J=2.1 Hz, J=8.2 Hz, 1H), 7.48 (d, J=2.0 Hz, 1H), 7.56 (d, J=8.2 Hz, 1H) 7.85-7.94 (bs, 1H).

¹³C-NMR (125 MHz, CDCl₃): δ (ppm)=17.0, 20.7, 24.3, 45.3, 46.2, 53.0, 67.9, 116.0 (d, J_{CF}=22.1 Hz, CH), 116.9 (d, J_{CF}=23.0 Hz, CH), 128.2 (CH), 130.9 (CH), 131.1 (CH), 133.1 (C_{q}), 133.4 (C_{q}), 136.9 (C_{q}), 152.5 (C_{q})

### (1S,3R,4S)-Quinuclidin-3-yl (4-fluoro-2-(thiophen-2-yl)phenyl)carbamate (EXP 18)

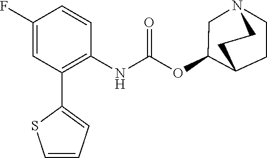 [SMILES: O=C(Nc1ccc(F)cc1-c1cccs1)O[C@H]1C[N@]2CC[C@H]1CC2]

Compound EXP 18 was prepared according to general procedure 2 using intermediate 2 and thiophene-2-boronic acid. Purification by preparative HPLC (column 1, eluent: CH₃CN/H₂O+0.1% HCO₂H) afforded the formate salt of EXP 18 (90 mg, 45%) as yellow brown oil.

¹H NMR (400 MHz, DMSO-d₆): δ (ppm)=9.05 (s, 1H), 7.65 (dd, J=5.1, 1.2 Hz, 1H), 7.54-7.50 (m, 2H), 7.33 (dd, J=8.7, 5.7 Hz, 1H), 7.18-7.12 (m, 2H), 4.63 (s, 1H), 3.29-3.05 (m, 1H), 2.99-2.54 (m, 5H), 2.06-1.75 (m, 1H), 1.71-1.18 (m, 4H).

¹³C-NMR (151 MHz, DMSO-d₆): δ (ppm)=160.15 (d, J=243.1 Hz), 154.46, 138.36, 133.04 (d, J=5.2 Hz), 130.86 (d, J=7.3 Hz), 130.06 (d, J=2.6 Hz), 127.48, 127.13, 126.85, 114.73 (d, J=23.6 Hz), 114.37 (d, J=22.3 Hz), 70.22, 54.33, 46.22, 45.27, 24.81, 23.06, 18.50.

HPLC (254 nm, System A): t_{R}=15.48 min; HPLC (254 nm, System B): t_{R}=14.33 min; ESI-MS: m/z=347.12 [M+H]⁺

### (1S,3R,4S)-3-(((4-Fluoro-2-(thiophen-2-yl)phenyl)carbamoyl)oxy)-1-methylquinuclidin-1-ium formate (EXP 19)

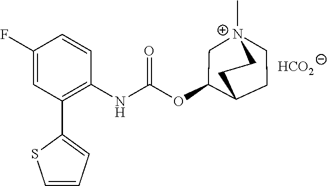

Compound EXP 19 was prepared according to general procedure 2 using EXP 18. Purification by preparative HPLC (column 1, eluent: CH₃CN/H₂O+0.1% HCO₂H) afforded EXP 19 (11 mg, 68%) as red brown oil.

¹H NMR (400 MHz, CD₃OD): δ (ppm)=8.52 (s, 1H), 7.57-7.51 (m, 1H), 7.51-7.28 (m, 3H), 7.19-7.05 (m, 2H), 5.14-4.95 (m, 1H), 3.84 (s, 1H), 3.59-3.38 (m, 4H), 3.00 (s, 3H), 2.53-2.20 (m, 2H), 2.19-1.80 (m, 4H).

¹³C-NMR (101 MHz, DMSO-d₆): δ (ppm)=165.25, 160.21 (d, J=244.4 Hz), 153.94, 138.25, 132.83 (d, J=7.4 Hz), 130.57, 129.70, 127.60, 127.31, 127.06, 114.94 (d, J=22.8 Hz), 114.51 (d, J=22.3 Hz), 67.42, 62.04, 55.66, 54.91, 50.82, 23.48, 20.53, 17.79.

HPLC (254 nm, System A): t_{R}=15.66 min, HPLC (254 nm, System B): t_{R}=14.57 min; ESI-MS: m/z=361.11 [M]⁺

### (1S,3R,4S)-Quinuclidin-3-yl (2-(3-bromothiophen-2-yl)-4-fluorophenyl)carbamate (EXP 20)

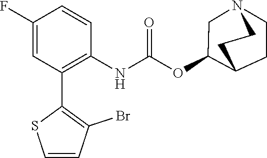 [SMILES: O=C(Nc1ccc(F)cc1-c1sccc1Br)O[C@H]1C[N@]2CC[C@H]1CC2]

Compound EXP 20 was prepared according to general procedure 2 using intermediate 2 and 3-bromothiophene-2-boronic acid N-methyliminodiacetic (MIDA) acid ester. Purification by preparative HPLC (column 1, eluent: CH₃CN/H₂O+0.1% CF₃CO₂H) afforded the trifluoroacetate salt of EXP 20 (11 mg, 18%) as yellow oil.

¹H NMR (600 MHz, CD₃OD): δ (ppm)=7.68-7.57 (m, 2H), 7.22 (td, J=8.5, 3.0 Hz, 1H), 7.16 (d, J=9.0 Hz, 1H), 7.13 (d, J=5.4 Hz, 1H), 5.05-5.00 (m, 1H), 3.75-3.68 (m, 1H), 3.46-3.34 (m, 1H), 3.31-3.13 (m, 4H), 2.40-2.29 (m, 1H), 2.26-2.12 (m, 1H), 2.11-2.00 (m, 2H), 2.00-1.81 (m, 2H).

¹³C NMR (101 MHz, CD₃OD -d₄): δ (ppm)=161.20 (d, J=243.7 Hz), 155.54, 135.05 (d, J=1.4 Hz), 133.47 (d, J=2.7 Hz), 131.87, 128.71, 119.33 (d, J=23.7 Hz), 117.40 (d, J=22.5 Hz), 111.81, 68.92, 54.59, 47.80, 46.88, 25.31, 21.06, 17.85.

HPLC (254 nm, System A): t_{R}=16.51 min; ESI-MS: m/z=426.94 [M+H]⁺

### (1S,3R,4S)-Quinuclidin-3-yl(4-fluoro-2-(3-methylthiophen-2-yl)phenyl)carbamate (EXP 21)

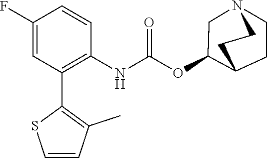 [SMILES: Cc1ccsc1-c1cc(F)ccc1NC(=O)O[C@H]1C[N@]2CC[C@H]1CC2]

Compound EXP 21 was prepared according to general procedure 2 using intermediate 2 and 3-methylthiophene-2-boronic acid pinacol ester. Purification by preparative HPLC (column 1, eluent: CH₃CN/H₂O+0.1% CF₃CO₂H) afforded the trifluoroacetate salt of EXP 21 (26 mg, 48%) as yellow oil.

¹H NMR (400 MHz, CD₃OD): δ (ppm)=7.68-7.56 (m, 1H), 7.42 (d, J=5.1 Hz, 1H), 7.15 (ddd, J=8.9, 8.1, 3.0 Hz, 1H), 7.07 (dd, J=9.0, 3.0 Hz, 1H), 6.97 (d, J=5.1 Hz, 1H), 4.99 (dt, J=7.3, 3.2 Hz, 1H), 3.72-3.63 (m, 1H), 3.33-3.12 (m, 5H), 2.37-2.29 (m, 1H), 2.11 (s, 3H), 2.09-1.79 (m, 5H).

¹³C NMR (101 MHz, CD₃OD): δ (ppm)=161.26 (d, J=245.3 Hz), 155.48, 137.14, 133.46 (d, J=2.7 Hz), 127.28, 119.26 (d, J=22.9 Hz), 116.53 (d, J=22.5 Hz), 68.91, 54.58, 47.75, 46.88, 25.31, J=22.6 Hz, 21.08, 17.81, 14.61.

HPLC (254 nm, System A): t_{R}=16.55 min; ESI-MS: m/z=361.05 [M+H]⁺

### (1S,3R,4S)-Quinuclidin-3-yl(4-fluoro-2-(4-methylthiophen-2-yl)phenyl)carbamate (EXP 22)

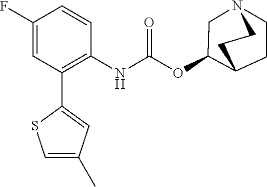 [SMILES: Cc1csc(-c2cc(F)ccc2NC(=O)O[C@H]2C[N@]3CC[C@H]2CC3)c1]

Compound EXP 22 was prepared according to general procedure 2 using intermediate 2 and 4-methylthiophene-2-boronic acid pinacol ester. Purification by preparative HPLC (column 1, eluent: CH₃CN/H₂O+0.1% CF₃CO₂H) afforded the trifluoroacetate salt of EXP 22 (23 mg, 43%) as yellow oil.

¹H NMR (600 MHz, CD₃OD): δ (ppm)=7.51-7.40 (m, 1H), 7.32-7.25 (m, 1H), 7.23-7.14 (m, 1H), 7.12-7.01 (m, 2H), 5.20-4.90 (m, 1H), 3.80-3.58 (m, 1H), 3.34-3.11 (m, 5H), 2.48-2.34 (m, 1H), 2.30 (s, 3H), 2.16-2.00 (m, 2H), 2.00-1.78 (m, 3H).

HPLC (254 nm, System A): t_{R}=16.39 min, HPLC (254 nm, System B): t_{R}=15.41 min; ESI-MS: m/z=361.07 [M+H]⁺

### (1S,3R,4S)-Quinuclidin-3-yl(4-fluoro-2-(5-methylthiophen-2-yl)phenyl)carbamate (EXP 23)

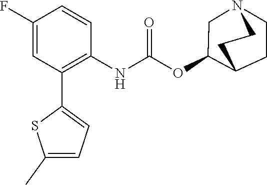 [SMILES: Cc1ccc(-c2cc(F)ccc2NC(=O)O[C@H]2C[N@]3CC[C@H]2CC3)s1]

Compound EXP 23 was prepared according to general procedure 2 using intermediate 2 and 5-methylthiophene-2-boronic acid pinacol ester. Purification by preparative HPLC (column 1, eluent: CH₃CN/H₂O+0.1% CF₃CO₂H) afforded the trifluoroacetate salt of EXP 23 (28 mg, 53%) as yellow oil.

¹H NMR (400 MHz, CD₃OD): δ (ppm)=7.48-7.38 (m, 2H), 7.28 (ddtt, J=9.8, 6.5, 2.2, 1.1 Hz, 2H), 7.15 (s, 1H), 7.04 (ddd, J=8.9, 7.9, 3.0 Hz, 1H), 6.82-6.77 (m, 1H), 5.03 (s, 1H), 3.80-3.60 (m, 1H), 3.45-3.33 (m, 2H), 3.29-3.17 (m, 3H), 2.50 (d, J=1.1 Hz, 3H), 2.45-2.35(m, 1H), 2.14-2.00 (m, 2H), 1.98-1.84 (m, 2H).

HPLC (254 nm, System A): t_{R}=16.41 min; ESI-MS: m/z=361.05 [M+H]⁺

### (1S,3R,4S)-Quinuclidin-3-yl (4-fluoro-2-(thiophen-3-yl)phenyl)carbamate (EXP 24)

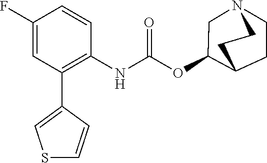 [SMILES: O=C(Nc1ccc(F)cc1-c1ccsc1)O[C@H]1C[N@]2CC[C@H]1CC2]

Compound EXP 24 was prepared according to general procedure 2 using intermediate 2 and thiophene-3-boronic acid. Purification by preparative HPLC (column 1, eluent: CH₃CN/H₂O+0.1% HCO₂H) afforded the formate salt of EXP 24 (99 mg, 49%) as colorless oil.

¹H NMR (600 MHz, CD₃OD): δ (ppm)=7.59-7.50 (m, 2H), 7.45-7.40 (m, 1H), 7.31-7.23 (m, 2H), 7.23-7.14 (m, 1H), 7.08 (ddd, J=8.8, 8.0, 3.0 Hz, 1H), 4.97 (s, 1H), 3.72-3.58 (m, 1H), 3.43-3.36 (m, 1H), 3.29-3.04 (m, 4H), 2.40-2.14 (m, 2H), 2.13-1.78 (m, 4H).

¹³C NMR (151 MHz, CD₃OD): δ (ppm)=163.64, 159.97 (d, J=242.3 Hz), 138.05, 134.67, 132.35, 130.67 (d, J=2.6 Hz), 128.73, 127.82, 126.12, 123.91, 115.90 (d, J=23.1 Hz), 114.15 (d, J=22.2 Hz), 69.01, 53.65, 45.95, 45.07, 24.35, 21.86, 17.74.

HPLC (254 nm, System A): t_{R}=15.39 min, HPLC (254 nm, System B): t_{R}=14.67 min; ESI-MS: m/z=347.11 [M+H]⁺

### (1S,3R,4S)-3-(((4-Fluoro-2-(thiophen-3-yl)phenyl)carbamoyl)oxy)-1-methylquinuclidin-1-ium formate (EXP 25)

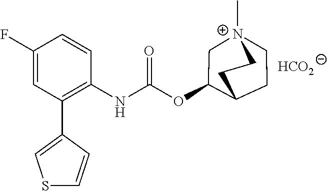

Compound EXP 25 was prepared according to general procedure 2 using EXP 24. Purification by preparative HPLC (column 1, eluent: CH₃CN/H₂O+0.1% HCO₂H) afforded EXP 25 (14 mg, 47%) as yellow oil.

¹H NMR (400 MHz, DMSO-d₆): δ (ppm)=9.36 (s, 1H), 7.84-7.71 (m, 1H), 7.70-7.60 (m, 1H), 7.49-7.27 (m, 3H), 7.25-7.09 (m, 1H), 4.94-4.75 (m, 1H), 3.80 (s, 1H), 3.56-3.18 (m, 5H), 2.95 (s, 3H), 2.30-2.01 (m, 2H), 1.89 (d, J=32.5 Hz, 3H).

¹³C-NMR (101 MHz, DMSO-d₆): δ (ppm)=159.99 (d, J=243.1 Hz), 153.69, 137.95, 134.39, 130.38 (d, J=2.7 Hz), 130.11, 127.93, 126.29, 124.11, 116.00 (d, J=23.0 Hz), 114.24 (d, J=22.2 Hz), 67.35, 62.03, 55.63, 54.89, 50.79, 23.37, 20.54, 17.79.

HPLC (254 nm, System A): t_{R}=15.38 min, HPLC (254 nm, System B): t_{R}=14.80 min; ESI-MS: m/z=361.09 [M]⁺

### (1S,3R,4S)-Quinuclidin-3-yl(4-fluoro-2-(5-methylthiophen-3-yl)phenyl)carbamate (EXP 26)

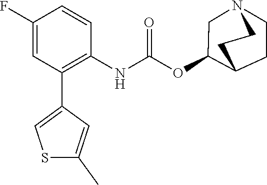 [SMILES: Cc1cc(-c2cc(F)ccc2NC(=O)O[C@H]2C[N@]3CC[C@H]2CC3)cs1]

Compound EXP 26 was prepared according to general procedure 2 using intermediate 2 and 5-methylthiophene-3-boronic acid pinacol ester. Purification by preparative HPLC (column 1, eluent: CH₃CN/H₂O+0.1% CF₃CO₂H) afforded the trifluoroacetate salt of EXP 26 (26 mg, 49%) as brown oil.

¹H NMR (600 MHz, CD₃OD): δ (ppm)=7.55-7.40 (m, 1H), 7.35-7.24 (m, 2H), 7.23-7.12 (m, 1H), 7.07 (ddd, J=8.8, 8.0, 3.0 Hz, 1H), 7.03-6.86 (m, 1H), 5.06-4.93 (m, 1H), 3.75-3.62 (m, 1H), 3.44-3.37 (m, 1H), 3.31-3.12 (m, 4H), 2.56-2.46 (m, 3H), 2.42-2.30 (m, 1H), 2.29-2.15 (m, 1H), 2.15-1.99 (m, 1H), 1.98-1.67 (m, 2H).

HPLC (254 nm, System A): t_{R}=16.28 min; ESI-MS: m/z=361.08 [M+H]⁺

### (1S,3R,4S)-Quinuclidin-3-yl (2-(3-bromofuran-2-yl)-4-fluorophenyl)carbamate (EXP 27)

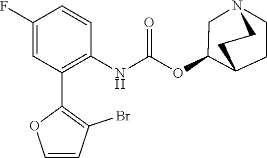 [SMILES: O=C(Nc1ccc(F)cc1-c1occc1Br)O[C@H]1C[N@]2CC[C@H]1CC2]

Compound EXP 27 was prepared according to general procedure 2 using intermediate 2 and 3-bromofurane-2-boronic acid MIDA ester. Purification by preparative HPLC (column 1, eluent: CH₃CN/H₂O+0.1% CF₃CO₂H) afforded the trifluoroacetate salt of EXP 27 (10 mg, 16%) as orange oil.

NMR (400 MHz, CD₃OD): δ (ppm)=7.68 (d, J=2.0 Hz, 1H), 7.65-7.56 (m, 1H), 7.47-7.40 (m, 1H), 7.36-7.30 (m, OH), 7.30-7.24 (m, 1H), 7.20 (ddd, J=8.9, 7.9, 3.0 Hz, 1H), 6.67 (d, J=2.0 Hz, 1H), 5.10-4.97 (m, 1H), 3.76-3.66 (m, 1H), 3.43-3.32 (m, 2H), 3.29-3.16 (m, 3H), 2.41-2.31 (m, 1H), 2.31-2.16 (m, 1H), 2.13-2.00 (m, 2H), 2.00-1.81 (m, 2H).

HPLC (254 nm, System A): t_{R}=16.30 min; ESI-MS: m/z=410.97 [M+H]⁺

### (1S,3R,4S)-quinuclidin-3-yl(4-fluoro-2-(5-methylfuran-2-yl)phenyl)carbamate (EXP 28)

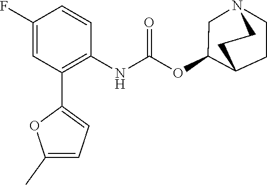 [SMILES: Cc1ccc(-c2cc(F)ccc2NC(=O)O[C@H]2C[N@]3CC[C@H]2CC3)o1]

Compound EXP 28 was prepared according to general procedure 2 using intermediate 2 and 5-methylfurane-2-boronic acid pinacol ester. Purification by preparative HPLC (column 1, eluent: CH₃CN/H₂O+0.1% CF₃CO₂H) afforded the trifluoroacetate salt of EXP 28 (17 mg, 33%) as yellow oil.

¹H NMR (600 MHz, CD₃OD): δ (ppm)=7.49 (s, 1H), 7.46-7.42 (m, 1H), 7.33-7.26 (m, 1H), 7.01 (ddd, J=8.8, 7.8, 3.0 Hz, 1H), 6.79-6.69 (m, 1H), 6.23-6.15 (m, 1H), 5.08 (s, 1H), 3.82-3.62 (m, 1H), 3.51-3.34 (m, 2H), 3.31-3.12 (m, 2H), 2.51-2.41 (m, 1H), 2.38 (d, J=1.1 Hz, 3H), 2.35-2.22 (m, 1H), 2.10 (dtt, J=18.6, 10.4, 3.4 Hz, 1H), 2.03-1.83 (m, 3H).

HPLC (254 nm, System A): t_{R}=16.17 min; ESI-MS: m/z=345.09 [M+H]⁺

### (1S,3R,4S)-Quinuclidin-3-yl(4-fluoro-2-(furan-3-yl)phenyl)carbamate (EXP 29)

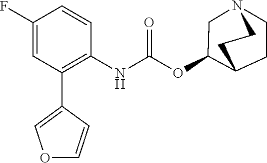 [SMILES: O=C(Nc1ccc(F)cc1-c1ccoc1)O[C@H]1C[N@]2CC[C@H]1CC2]

Compound EXP 29 was prepared according to general procedure 2 using intermediate 2 and furane-3-boronic acid. Purification by preparative HPLC (column 1, eluent: CH₃CN/H₂O+0.1% CF₃CO₂H) afforded the trifluoroacetate salt of EXP 29 (35 mg, 90%) as yellow oil.

¹H NMR (600 MHz, DMSO-d6): δ (ppm)=8.60 (s, 1H), 7.95 (dd, J=1.6, 0.9 Hz, 1H), 7.71 (t, J=1.7 Hz, 1H), 7.33 (ddd, J=11.9, 9.3, 4.3 Hz, 2H), 7.08 (td, J=8.5, 3.1 Hz, 1H), 6.83 (dd, J=2.0, 0.9 Hz, 1H), 4.60-4.56 (m, 1H), 3.09-3.02 (m, 1H), 2.80-2.54 (m, 4H), 2.45 (s, 1H), 1.86 (q, J=3.4 Hz, 1H), 1.71-1.62 (m, 1H), 1.61-1.55 (m, 1H), 1.45 (dddt, J=13.1, 10.3, 5.7, 2.8 Hz, 1H), 1.29 (ddt, J=16.6, 9.4, 3.8 Hz, 1H).

HPLC (254 nm, System A): t_{R}=14.91 min; ESI-MS: m/z=331.14 [M+H]⁺

### (1S,3R,4S)-Quinuclidin-3-yl (4-fluoro-2-(thiazol-5-yl)phenyl)carbamate (EXP 30)

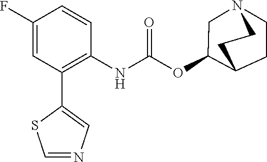 [SMILES: O=C(Nc1ccc(F)cc1-c1cncs1)O[C@H]1C[N@]2CC[C@H]1CC2]

Compound EXP 30 was prepared according to general procedure 2 using intermediate 2 and thiazole-5-boronic acid MIDA ester. Purification by preparative HPLC (column 1, eluent: CH₃CN/H₂O+0.1% CF₃CO₂H) afforded the trifluoroacetate salt of EXP 30 (26 mg, 49%) as colorless oil.

¹H NMR (600 MHz, CD₃OD): δ (ppm)=10.58-9.33 (m, 1H), 8.87-8.22 (m, 1H), 7.58-7.34 (m, 2H), 7.24-7.15 (m, 1H), 5.09-4.97 (m, 1H), 3.82-3.63 (m, 1H), 3.48-3.33 (m, 2H), 3.31-3.16 (m, 3H), 2.50-2.34 (m, 1H), 2.34-2.18 (m, 1H), 2.14-2.02 (m, 1H), 2.02-1.82 (m, 2H).

¹³C NMR (101 MHz, CD₃OD): δ (ppm)=162.44 (d, J=247.0 Hz), 156.05, 131.99 (d, J=1.6 Hz), 131.32 (d, J=2.2 Hz), 131.00, 130.37, 117.75 (d, J=23.5 Hz), 117.22 (d, J=22.7 Hz), 69.07, 54.70, 47.82, 46.89, 25.41, 21.08, 17.87.

HPLC (254 nm, System A): t_{R}=13.59 min; ESI-MS: m/z=348.04 [M+H]⁺

### (1S,3R,4S)-Quinuclidin-3-yl(4-fluoro-2-(pyridin-4-yl)phenyl)carbamate (EXP 31)

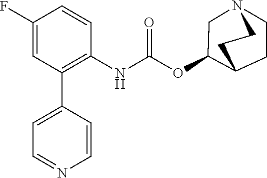 [SMILES: O=C(Nc1ccc(F)cc1-c1ccncc1)O[C@H]1C[N@]2CC[C@H]1CC2]

Compound EXP 31 was prepared according to general procedure 2 using intermediate 2 and pyridine-4-boronic acid. Purification by preparative HPLC (column 1, eluent: CH₃CN/H₂O+0.1% CF₃CO₂H) afforded the trifluoroacetate salt of EXP 31 (28 mg, 70%) as colorless oil.

¹H NMR (600 MHz, CD₃OD): δ (ppm)=9.13-8.38 (m, 2H), 7.68-7.52 (m, 2H), 7.49 (dd, J=8.6, 5.1 Hz, 1H), 7.26 7.16 (m, 2H), 4.97 4.89 (m, 1H), 3.65 3.55 (m, 1H), 3.30-3.00 (m, 5H), 2.25-2.17 (m, 1H), 2.15-2.05 (m, 1H), 2.04-1.94 (m, 1H), 1.90-1.75 (m, 2H).

¹³C NMR (101 MHz, CD₃OD): δ (ppm)=162.32 (d, J=245.5 Hz), 155.80, 150.28, 148.79, 138.27, 131.71 (d, J=3.2 Hz), 130.18, 117.69 (d, J=23.9 Hz), 117.24 (d, J=22.5 Hz), 69.24, 54.49, 47.59, 46.72, 25.45, 21.33, 17.99.

HPLC (254 nm, System A): t_{R}=10.44 min; ESI-MS: m/z=342.15 [M+H]⁺

### (1S,3R,4S)-Quinuclidin-3-yl(4-fluoro-2-(pyrimidin-5-yl)phenyl)carbamate (EXP 32)

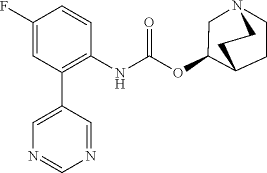 [SMILES: O=C(Nc1ccc(F)cc1-c1cncnc1)O[C@H]1C[N@]2CC[C@H]1CC2]

Compound EXP 32 was prepared according to general procedure 2 using intermediate 2 and pyrimidine-4-boronic acid and Na₂CO₃ (3 eq). Purification by preparative HPLC (column 1, eluent: CH₃OH/H₂O+0.1% HCO₂H) afforded the trifluoroacetate salt of EXP 32 (73 mg, 73%) as pale green lyophilisate.

¹H NMR (400 MHz, DMSO-d6): δ (ppm)=9.40-9.10 (m, 2H), 8.90-8.77 (m, 2H), 7.58-7.22 (m, 3H), 4.70-4.55 (m, 1H), 3.42-3.19 (m, 1H), 3.10-2.58 (m, 5H), 1.99-1.87 (m, 1H), 1.85-1.65 (m, 2H), 1.63-1.42 (m, 2H).

¹³C NMR (151 MHz, DMSO-d6): δ (ppm)=159.90 (d, J=243.6 Hz), 157.08, 156.02, 153.81, 132.71 (d, J=8.2 Hz), 131.89, 131.46 (d, J=2.6 Hz), 129.02, 116.77 (d, J=23.6 Hz), 115.98 (d, J=22.2 Hz), 69.31, 53.13, 45.57, 44.70, 24.31, 21.82, 17.77.

HPLC (254 nm, System A): t_{R}=12.79 min, HPLC (254 nm, System B): t_{R}=11.96 min; ESI-MS: m/z=343.10 [M+H]⁺

### (1R,3R,5S)-8-Methyl-8-azabicyclo[3.2.1]octan-3-yl(4-fluoro-2-(thiophen-2-yl)phenyl)carbamate (EXP 33)

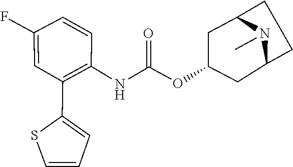 [SMILES: CN1[C@@H]2CC[C@H]1C[C@H](OC(=O)Nc1ccc(F)cc1-c1cccs1)C2]

To a solution of intermediate 5 in 1,4-dioxane (4 mL) were added thiophene-2-boronic acid (2 eq), 1′1[bis(diphenylphosphino)ferrocene]dichloropalladium(II) (0.2 eq) and Na₂CO₃ (8 eq) in a microwave tube. After addition of H₂O (1 mL) the tube was sealed and the reaction mixture was stirred at 90° C. for 16 h. The mixture was allowed to cool to room temperature, was filtered through celite and MgSO₄ and the filter was repeatedly washed with ethyl acetate.

After removing the solvent under reduced pressure with a rotary evaporator, the crude residue was dissolved in saturated aqueous Na₂CO₃ solution. The aqueous layer was extracted three times with CHCl₃ and the combined organic layers were washed with saturated, aqueous NaCl solution and dried over Na₂SO₄. The organic solvent was removed under reduced pressure and the residue was purified by preparative HPLC (column 1, eluent: CH₃OH/H₂O+0.1% HCO₂H) to afford the formate salt of EXP 33 (101 mg, 86%) as yellow oil.

¹H NMR (400 MHz, DMSO-d₆): δ (ppm)=9.00 (s, 1H), 7.65 (dd, J=5.1, 1.0 Hz, 1H), 7.57-7.51 (m, 2H), 7.34 (dd, J=8.7, 5.7 Hz, 1H), 7.19-7.12 (m, 2H), 4.75 (s, 1H), 3.55-3.10 (m, 2H), 2.39 (s, 3H), 2.28-1.89 (m, 4H), 1.86-1.28 (m, 4H).

¹³C-NMR (151 MHz, DMSO-d₆): δ (ppm)=160.19 (d, J=241.7 Hz), 154.10, 138.31, 138.30, 130.83, 130.03, 127.53, 127.14, 126.92, 114.67, 114.37 (d, J=22.3 Hz), 65.71, 59.59, 38.21, 34.52, 24.44.

HPLC (254 nm, System A): t_{R}=15.59 min, HPLC (254 nm, System B): t_{R}=14.64 min; ESI-MS: m/z=361.10 [M+H]⁺

### (1R,3S,5S)-8-Methyl-8-azabicyclo[3.2.1]oct-6-en-3-yl (4-fluoro-2-(thiophen-2-yl)phenyl)carbamate (EXP 34)

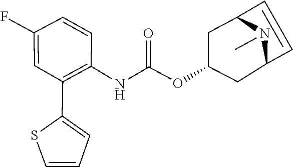 [SMILES: CN1[C@@H]2C=C[C@H]1C[C@H](OC(=O)Nc1ccc(F)cc1-c1cccs1)C2]

To a solution of compound 3 in dry toluene (8 mL) were added dry dimethyl formamide (10 μL) and SOCl₂ (0.5 mL) under argon atmosphere and the mixture was stirred under reflux conditions for 16 h. After the reaction mixture was allowed to cool to room temperature, excess SOCl₂ was removed under reduced pressure. NaN₃ (2.5 eq) was added under argon atmosphere and the mixture was stirred at room temperature for 40 minutes and subsequently heated to 90° C. for 16 h. After cooling to room temperature intermediate 9 (1.5 eq) in dry DMSO (dimethyl sulfoxide) (2.6 mL) was added under argon atmosphere and the reaction was stirred again at 120° C. for 16 h. After cooling to room temperature, saturated, aqueous NaHCO₃ solution was added and the aqueous layer was extracted three times with CHCl₃. The combined organic layers were washed with saturated, aqueous NaCl solution and dried over Na₂SO₄. The solvent was removed under reduced pressure in a rotary evaporator and the crude residue was purified by preparative HPLC (column 1, eluent: CH₃CN/H₂O +0.1% HCO₂H) to afford the formate salt of EXP 34 (31 mg, 32%) as a colorless oil.

¹H NMR (600 MHz, DMSO-d6): δ (ppm)=8.44-8.32 (m, 1H), 7.62-7.60 (m, 1H), 7.47-7.41 (m, 2H), 7.29 (dd, J=8.8, 5.7 Hz, 1H), 7.15-7.09 (m, 2H), 5.87-5.65 (m, 2H), 4.72 (t, J=6.2 Hz, 1H), 3.35-3.30 (m, 2H), 2.20-2.13 (m, 3H), 2.07 (ddd, J=14.7, 6.2, 3.6 Hz, 2H), 1.52 (d, J=14.6 Hz, 2H).

HPLC (254 nm, System A): t_{R}=15.70 min, HPLC (254 nm, System B): t_{R}=14.70 min; ESI-MS: m/z=358.96 [M+H]⁺

### (1R,2R,4S,5S,7S)-9-Methyl-3-oxa-9-azatricyclo[3.3.1.0^{2,4}]nonan-7-yl(4-fluoro-2-(thiophen-2-yl)phenyl)carbamate (EXP 35)

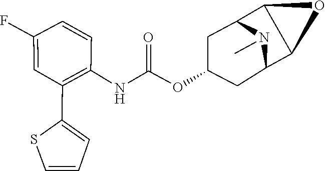 [SMILES: CN1[C@@H]2C[C@H](OC(=O)Nc3ccc(F)cc3-c3cccs3)C[C@H]1[C@H]1O[C@H]12]

Compound EXP 35 was prepared according to step 2 of general procedure 4 using a solution of intermediate 31 and the bicyclic amino alcohol scopine stirring the reaction mixture at 40° C. for 16 h. Purification by preparative HPLC (column 1, eluent: CH₃CN/H₂O+0.1% HCO₂H) afforded the formate salt of EXP 35 (2.1 mg, 2.3%) as yellow oil.

¹H NMR (600 MHz, DMSO-d₆): δ (ppm)=8.94-8.72 (m, 1H), 8.50-8.24 (m, 1H), 7.68 (d, J=5.1 Hz, 1H), 7.63-7.45 (m, 2H), 7.32 (dd, J=8.7, 5.6 Hz, 1H), 7.21-7.10 (m, 2H), 4.79-4.62 (m, 1H), 3.88-3.58 (m, 2H), 3.07 (s, 3H), 2.41-2.24 (m, 2H), 2.06-1.79 (m, 2H), 1.63-1.17 (m, 2H).

ESI-MS: m/z=374.96 [M+H]⁺

### (1R,2R,4S,5S,7S)-7-(((4-Fluoro-2-(thiophen-2-yl)phenyl)carbamoyl)oxy)-9,9-dimethyl-3-oxa-9-azatricyclo[3.3.1.0^{2,4}]nonan-9-ium formate (EXP 36)

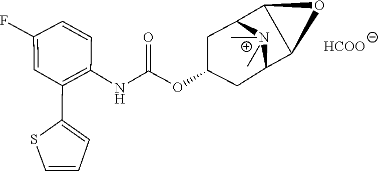

To a solution of intermediate 3 in dry toluene (4 mL) were added dry dimethyl formamide (10 μL) and SOCl₂ (0.5 mL) under argon atmosphere and the mixture was stirred under reflux conditions for 16 h. After the reaction was allowed to cool to room temperature SOCl₂ was removed under reduced pressure. NaN₃ (2.5 eq) was added under argon atmosphere and the mixture was stirred at room temperature for 40 minutes and subsequently heated to 90° C. for 16 h. After cooling to room temperature a solution of intermediate 10 (1.5 eq) in dry DMSO (2.6 mL) was added under argon atmosphere and the reaction was stirred again at 60° C. for 24 h. After cooling to room temperature the reaction was acidified with aqueous HCl solution (1M) and the aqueous layer was washed three times with CHCl₃. The aqueous layer was neutralized with aqueous NaOH solution (1M) and organic solvent residues were removed under reduced pressure. Purification by preparative HPLC (column 1, eluent: CH₃CN/H₂O+0.1% HCO₂H) yielded EXP 36 (15.1 mg, 9%) as pale yellow lyophilisate.

¹H NMR (400 MHz, DMSO-d₆): δ (ppm)=9.05 (s, 1H), 7.67 (d, J=5.0 Hz, 1H), 7.60-7.48 (m, 2H), 7.39 (dd, J=8.8, 5.6 Hz, 1H), 7.30-7.12 (m, 2H), 4.91 (s, 1H), 4.25-4.01 (m, 4H), 3.29 (s, 3H), 3.04 (s, 3H), 2.67-2.56 (m, 2H), 1.95 (s, 2H).

¹³C NMR (151 MHz, DMSO-d₆): δ (ppm)=163.03 (d, J=254.2 Hz), 153.46, 138.14, 136.35, 133.04 (d, J=9.6 Hz), 130.45, 129.72, 127.73, 127.26, 127.10, 115.06 (d, J=21.5 Hz), 114.44 (d, J=22.5 Hz), 64.85, 61.44, 55.51, 52.52, 46.95, 28.76.

HPLC (254 nm, System A): t_{R}=15.49 min, HPLC (254 nm, System B): t_{R}=14.83 min; ESI-MS: m/z=388.95 [M]⁺

### (1S,3R,4S)-Quinuclidin-3-yl(2-(benzo[b]thiophen-2-yl)-4-fluorophenyl)carbamate (EXP 37)

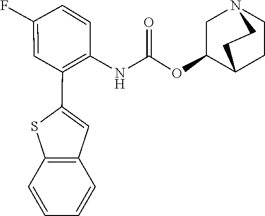 [SMILES: O=C(Nc1ccc(F)cc1-c1cc2ccccc2s1)O[C@H]1C[N@]2CC[C@H]1CC2]

Compound EXP 37 was prepared according to general procedure 2 using a solution of intermediate 2 and benzo[b]thien-2-ylboronic acid. Purification by preparative HPLC (column 1, eluent: CH₃CN/H₂O+0.1% CF₃CO₂H) afforded the trifluoroacetate salt of EXP 37 as pale orange lyophilisate.

ESI-MS: m/z=397.10 [M+H]⁺

### (1S,3R,4S)-Quinuclidin-3-yl(2-(benzo[b]thiophen-3-yl)-4-fluorophenyl)carbamate (EXP 38)

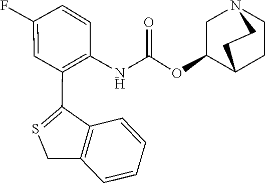

Compound EXP 38 was prepared according to general procedure 2 using a solution intermediate 2 and benzo[b]thien-3-ylboronic acid. Purification by preparative HPLC (column 1, eluent: CH₃CN/H₂O+0.1% CF₃CO₂H) afforded the trifluoroacetate salt EXP 38 (17 mg, 67%) as pale colorless oil.

HPLC (254 nm, System A): t_{R}=17.25 min; ESI-MS: m/z=397.12 [M+H]⁺

### (1S,3R,4S)-Quinuclidin-3-yl(2-(benzofuran-2-yl)-4-fluorophenyl)carbamate (EXP 39)

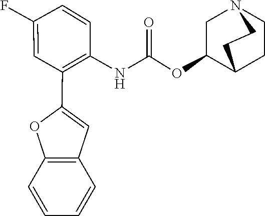 [SMILES: O=C(Nc1ccc(F)cc1-c1cc2ccccc2o1)O[C@H]1C[N@]2CC[C@H]1CC2]

Compound EXP 39 was prepared according to general procedure 2 using a solution of intermediate 2 and benzofurane-2-boronic acid MIDA ester. Purification by preparative HPLC (column 1, eluent: CH₃CN/H₂O+0.1% CF₃CO₂H) afforded the trifluoroacetate salt EXP 39 as pale yellow oil.

HPLC (254 nm, System A): t_{R}=16.05 min; ESI-MS: m/z=381.10 [M+H]⁺

### (1S,3R,4S)-Quinuclidin-3-yl(4-fluoro-2-(1H-inden-3-yl)phenyl)carbamate (EXP 40)

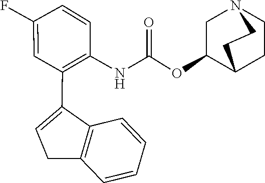 [SMILES: O=C(Nc1ccc(F)cc1C1=CCc2ccccc21)O[C@H]1C[N@]2CC[C@H]1CC2]

Compound EXP 40 was prepared according to general procedure 2 using a solution of intermediate 2 and 1H-indene-3-boronic acid. Purification by preparative HPLC (column 1, eluent: CH₃CN/H₂O+0.1% CF₃CO₂H) afforded the trifluoroacetate salt EXP 40 (28 mg, 50%) as orange lyophilisate.

HPLC (254 nm, System A): t_{R}=17.23 min; ESI-MS: m/z=379.17 [M+H]⁺

### (1S,3R,4S)-Quinuclidin-3-yl (4-fluoro-2-(phenylethynyl)phenyl)carbamate (EXP 41)

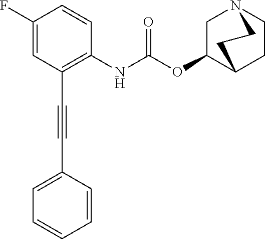 [SMILES: O=C(Nc1ccc(F)cc1C#Cc1ccccc1)O[C@H]1C[N@]2CC[C@H]1CC2]

Compound EXP 41 was prepared according to general procedure 3 using a solution of intermediate 2 and phenylacetylene. Purification by preparative HPLC (column 1, eluent: CH₃CN/H₂O+0.1% CF₃CO₂H) afford the trifluoroacetate salt EXP 41 (28 mg, 53%) as white lyophilisate.

HPLC (254 nm, System A): t_{R}=17.61 min; ESI-MS: m/z=365.16 [M+H]⁺

### (1S,3R,4S)-Quinuclidin-3-yl (2-(cyclohexylethynyl)-4-fluorophenyl)carbamate (EXP 42)

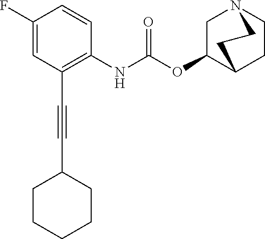 [SMILES: O=C(Nc1ccc(F)cc1C#CC1CCCCC1)O[C@H]1C[N@]2CC[C@H]1CC2]

Compound EXP 42 was prepared according to general procedure 3 using a solution of intermediate 2 and cyclohexylacetylene. Purification by preparative HPLC (column 1, eluent: CH₃CN/H₂O+0.1% CF₃CO₂H) afforded the trifluoroacetate salt EXP 42 (38 mg, 70%) as white lyophilisate.

¹H NMR (600 MHz, CD₃OD): δ (ppm)=7.80-7.66 (m, 1H), 7.60-7.54 (m, 2H), 7.44-7.38 (m, 3H), 7.28 (dd, J=8.8, 3.0 Hz, 1H), 7.14 (ddd, J=9.0, 8.1, 3.0 Hz, 1H), 5.15-5.08 (m, 1H), 3.80-3.71 (m, 1H), 3.44-3.35 (m, 2H), 3.32 (s, 1H), 3.30-3.20 (m, 2H), 2.46-2.39 (m, 1H), 2.34-2.14 (m, 1H), 2.11-2.02 (m, 1H), 2.00-1.80 (m, 2H).

¹³C NMR (151 MHz, CD₃OD): δ (ppm)=160.70 (d, J=238.1 Hz), 155.19, 136.16 (d, J=2.8 Hz), 132.72, 130.27, 129.75, 123.71, 119.38 (d, J=24.4 Hz), 117.34 (d, J=22.7 Hz), 96.72, 85.12, 69.17, 54.57, 47.71, 46.89, 25.42, 21.12, 17.87.

HPLC (254 nm, System A): t_{R}=18.68 min; ESI-MS: m/z=371.19 [M+H]⁺

### (1R,2R,4S,5S,7S)-7-(((4-Fluoro-2-(5-methylthiophen-2-yl)phenyl)carbamoyl)oxy)-9,9-dimethyl-3-oxa-9-azatricyclo[3.3.1.0^{2,4}]nonan-9-ium formate (EXP 43)

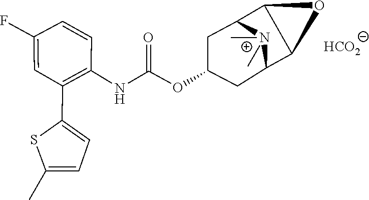

Compound EXP 43 was prepared according to step 2 of general procedure 4 using a solution of intermediate 32 and intermediate 10 and stirring the reaction mixture at 60° C. for 16 h. Purification by preparative HPLC (column 1, eluent: CH₃CN/H₂O +0.1% HCO₂H) afforded EXP 43 (67.1 mg, 62%) as yellow oil.

¹H NMR (400 MHz, Methanol-d₄): δ (ppm)=8.54 (s, 1H), 7.51-7.08 (m, 3H), 7.04 (td, J=8.3, 2.9 Hz, 1H), 6.85-6.77 (m, 1H), 5.10-4.94 (m, 1H), 4.47-3.71 (m, 4H), 3.39 (s, 3H), 3.10 (s, 3H), 2.87-2.57 (m, 2H), 2.50 (s, 3H), 2.21-1.64 (m, 2H).

¹³C NMR (151 MHz, Methanol-d₄): δ (ppm)=170.04, 162.20 (d, J=245.4 Hz), 155.59, 142.75, 137.62 (d, J=2.2 Hz), 134.32, 130.91, 130.57, 128.16, 126.89, 116.60, 115.41 (d, J=22.7 Hz), 67.10, 63.41, 57.14, 55.26, 48.15, 30.36, 15.13.

HPLC (254 nm, System A): t_{R}=16.21 min, HPLC (254 nm, System B): t_{R}=15.36 min; ESI-MS: m/z=403.08[M]⁺

### (1R,2R,4S,5S,7S)-7-(((4-Fluoro-2-(4-methylthiophen-2-yl)phenyl)carbamoyl)oxy)-9,9-dimethyl-3-oxa-9-azatricyclo[3.3.1.0^{2,4}]nonan-9-ium formate (EXP 44)

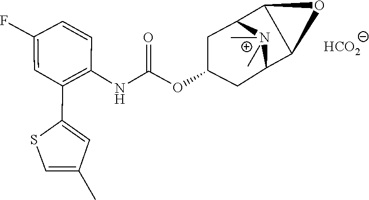

Compound EXP 44 was prepared according to step 2 of general procedure 4 using a solution of intermediate 33 and intermediate 10 and stirring the reaction mixture at 60° C. for 16 h. Purification by preparative HPLC (column 1, eluent: CH₃CN/H₂O+0.1% HCO₂H) afforded EXP 44 (47.7 mg, 37%) as yellow oil.

¹H NMR (400 MHz, Methanol-d₄): δ (ppm)=8.54 (s, 1H), 7.46-7.12 (m, 3H), 7.12-7.09 (m, 1H), 7.06 (td, J=8.3, 2.9 Hz, 1H), 5.12-4.94 (m, 1H), 4.34-3.78 (m, 4H), 3.39 (s, 3H), 3.10 (s, 3H), 2.87-2.58 (m, 2H), 2.28 (s, 3H), 2.24-1.78 (m, 2H).

¹³C NMR (151 MHz, Methanol-d₄): δ (ppm)=170.00, 162.21 (d, J=244.9 Hz), 155.57, 139.74 (d, J=2.3 Hz), 139.30, 134.26, 131.04, 130.58, 130.43, 123.46, 116.82 (d, J=20.6 Hz), 115.68 (d, J=22.6 Hz), 67.14, 63.43, 57.16, 55.28, 48.16, 30.37, 15.67.

HPLC (254 nm, System A): t_{R}=16.77 min, HPLC (254 nm, System B): t_{R}=15.44 min; ESI-MS: m/z=403.04 [M]⁺.

### (1R,2R,4S,5S,7S)-7-(((4-Fluoro-2-(3-methylthiophen-2-yl)phenyl)carbamoyl)oxy)-9,9-dimethyl-3-oxa-9-azatricyclo[3.3.1.0^{2,4}]nonan-9-ium formate (EXP 45)

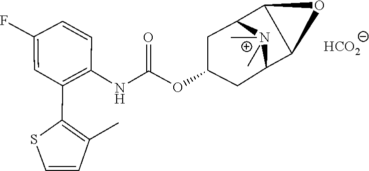

Compound EXP 45 was prepared according to step 2 of general procedure 4 using a solution of intermediate 34 and intermediate 10 and stirring the reaction mixture at 60° C. for 16 h. Purification by preparative HPLC (column 1, eluent: CH₃CN/H₂O +0.1% HCO₂H) afforded EXP 45 (58.8 mg, 54%) as yellow oil.

¹H NMR (400 MHz, Methanol-d₄): δ (ppm)=8.56 (s, 1H), 7.57 (dd, J=8.8, 5.4 Hz, 1H), 7.43 (d, J=5.1 Hz, 1H), 7.15 (ddd, J=8.9, 8.1, 3.0 Hz, 1H), 7.08 (dd, J=9.0, 3.0 Hz, 1H), 6.98 (d, J=5.1 Hz, 1H), 4.98 (t, J=6.0 Hz, 1H), 4.09 (d, J=2.48 1.6 Hz, 2H), 3.96 (s, 2H), 3.38 (s, 3H), 3.10 (s, 3H), 2.73 (ddd, J=17.7, 6.1, 4.1 Hz, 2H), 2.11 (s, 3H), 2.01 (d, J=17.5 Hz, 2H).

¹³C NMR (151 MHz, Methanol-d₄): δ (ppm)=170.20, 161.34 (d, J=245.0 Hz), 155.14, 137.13, 133.54 (d, J=1.8 Hz), 133.49 (d, J=3.0 Hz), 131.25, 128.73, 126.28, 119.20 (d, S=23.0 Hz), 116.46 (d, J=22.5 Hz), 67.08, 63.35, 57.11, 55.21, 48.15, 30.23, 14.63.

HPLC (254 nm, System A): t_{R}=17.10 min, HPLC (254 nm, System B): t_{R}=15.65 min; ESI-MS: m/z=403.06 [M]⁺

### (1R,2R,4S,5S,7S)-7-(((4-Fluoro-2-(thiophen-3-yl)phenyl)carbamoyl)oxy)-9,9-dimethyl-3-oxa-9-azatricyclo[3.3.1.0^{2,4}]nonan-9-ium formate (EXP 46)

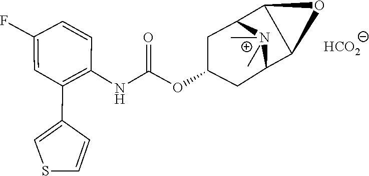

Compound EXP 46 was prepared according to step 2 of general procedure 4 using a solution of intermediate 35 and intermediate 10 and stirring the reaction mixture at 60° C. for 16 h. Purification by preparative HPLC (column 1, eluent: CH₃CN/H₂O+0.1% HCO₂H) afforded EXP 46 (10.6 mg, 18%) as yellow oil.

HPLC (254 nm, System A): t_{R}=15.69 min, HPLC (254 nm, System B): t_{R}=15.00 min; ESI-MS: m/z=389.01 [M]⁺

### (1R,2R,4S,5S,7S)-7-(((2-(Benzo[b]thiophen-2-yl)-4-fluorophenyl)carbamoyl)oxy)-9,9-dimethyl-3-oxa-9-azatricyclo[13.3.1.0^{2,4}]nonan-9-ium formate (EXP 47)

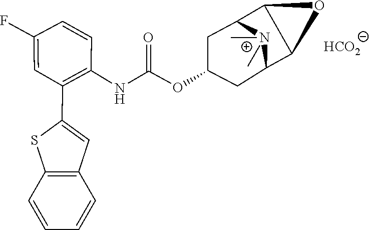

Compound EXP 47 was prepared according to step 2 of general procedure 4 using a solution of intermediate 36 and intermediate 10 and stirring the reaction mixture at 60° C. for 16 h. Purification by preparative HPLC (column 1, eluent: CH₃CN/H₂O+0.1% HCO₂H) afforded EXP 47 (11 mg, 20%) as yellow oil.

¹H NMR (400 MHz, Methanol-d₄): δ (ppm)=8.55 (s, 1H), 7.94-7.82 (m, 2H), 7.73-7.53 (m, 1H), 7.53-7.43 (m, 2H), 7.43-7.32 (m, 2H), 7.16 (ddd, J=8.8, 7.9, 3.0 Hz, 1H), 5.05-4.95 (m, 1H), 4.27-3.81 (m, 4H), 3.37 (s, 2H), 3.07 (s, 3H), 2.82-2.56 (m, 2H), 2.18-1.83 (m, 2H).

¹³C NMR (151 MHz, Methanol-d₄): δ (ppm)=170.31, 160.60 (d, J=244.9 Hz), 155.54, 141.66, 141.14, 140.42, 131.88, 130.54, 126.18, 125.91, 125.06, 125.01, 123.07, 117.77, 116.73 (d, J=22.7 Hz), 67.15, 63.50, 57.16, 55.26, 48.14, 30.33.

HPLC (254 nm, System A): t_{R}=17.78 min, HPLC (254 nm, System B): t_{R}=16.33 min; ESI-MS: m/z=439.04 [M]⁺

### (1R,2R,4S,5S,7S)-7-(((2-(Benzo[b]thiophen-3-yl)-4-fluorophenyl)carbamoyl)oxy)-9,9-dimethyl-3-oxa-9-azatricyclo[3.3.1.0^{2,4}]nonan-9-ium formate (EXP 48)

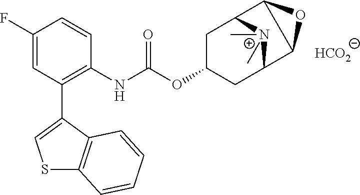

Compound EXP 48 was prepared according to step 2 of general procedure 4 using a solution of intermediate 37 and intermediate 10 and stirring the reaction mixture at 60° C. for 16 h. Purification by preparative HPLC (column 1, eluent: CH₃CN/H₂O+0.1% HCO₂H) afforded EXP 48 (25.1 mg, 25%) as yellow oil.

¹H NMR (400 MHz, Methanol-d₄): δ (ppm)=8.54 (s, 1H), 8.03-7.97 (m, 1H), 7.63 (s, 1H), 7.54 (dd, J=8.6, 5.3 Hz, 1H), 7.50-7.34 (m, 3H), 7.28-7.16 (m, 2H), 4.86-4.79 (m, 1H), 3.98-3.90 (m, 2H), 3.34-3.31 (m, 2H), 3.31 (s, 3H), 3.03 (s, 3H), 2.60 (dt, J=17.5, 5.6 Hz, 2H), 1.74 (d, J=17.4 Hz, 2H).

¹³C NMR (151 MHz, Methanol-d₄): δ (ppm)=170.04, 162.01 (d, J=245.0 Hz), 155.19, 141.62, 139.19, 135.13, 134.83, 132.95 (d, J=3.1 Hz), 129.46, 127.51, 125.78, 125.59, 124.13, 123.98, 118.64 (d, J=23.1 Hz), 116.43 (d, J=22.6 Hz), 66.97, 63.23, 57.10, 54.90, 48.12, 30.05.

HPLC (254 nm, System A): t_{R}=17.87 min, HPLC (254 nm, System B): t_{R}=16.30 min; ESI-MS: m/z=439.04 [M]⁺

### (1R,2R,4S,5S,7S)-9-Methyl-3-oxa-9-azatricyclo[3.3.1.0^{2,4}]nonan-7-yl (5-fluoro-[1,1′-biphenyl]-2-yl)carbamate (EXP 49)

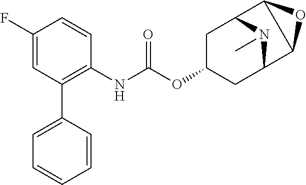 [SMILES: CN1[C@@H]2C[C@@H](OC(=O)Nc3ccc(F)cc3-c3ccccc3)C[C@H]1[C@@H]1O[C@@H]12]

Intermediate 11 (0.22 mmol, 1.0 eq.) was dissolved in dry pyridine (2 mL) at room temperature. Intermediate 40 (0.24 mmol, 1.1 eq.) was added portion-wise while stirring. The mixture was then heated to 60° C. and stirred for 24 h. Then, the mixture was allowed to cool to room temperature and the solvent was then removed under reduced pressure. Purification by preparative HPLC (eluent: CH₃CN/H₂O+0.1% HCO₂H) afforded the hydrochloride of EXP 49 (6.8 mg, 8%) as a white solid.

¹H NMR (400 MHz, CDCl₃): δ (ppm)=7.91 (s, 1H), 7.58-7.44 (m, 3H), 7.39-7.32 (m, 2H), 7.09 (ddd, J=9.0, 8.0, 3.0 Hz, 1H), 7.01 (dd, J=8.9, 3.0 Hz, 1H), 6.37 (s, 1H), 5.02 (s, 1H), 3.56 (s, 2H), 3.49 (s, 2H), 2.74 (s, 5H), 1.82 (d, J=14.9 Hz, 2H).

¹³C NMR (101 MHz, CDCl₃): δ (ppm)=152.52, 137.07, 130.14, 129.15, 128.91, 128.54, 116.94, 116.71, 115.19, 114.97, 83.48, 73.23, 70.04, 66.19, 59.42, 55.68, 55.12, 53.07, 42.93, 41.59, 30.69, 29.67.

ESI-MS: m/z=369 [M+H]⁺, HR EI-MS: calculated for C₂₁H₂₂FN₂O₃ [M]⁺: 369,1609, found: 369.1609.

Synthesis and Characterization of Reference Compounds:

(1R,2R,4S,5S,7S)-9,9-Dimethyl-7-(((2-(thiophen-2-yl)phenyl)carbamoyl)oxy)-3-oxa-9-azatricyclo[3.3.1.0^{2,4}]nonan-9-ium formate (REF 1)

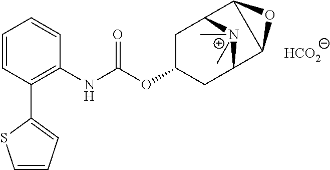

Compound EXP 49 was prepared according to step 2 of general procedure 4 using a solution of intermediate 38 and intermediate 10 and stirring the reaction mixture at 60° C. for 16 h. Purification by preparative HPLC (column 1, eluent: CH₃CN/H₂O +0.1% HCO₂H) afforded REF 1 (38.5 mg, 36%) as yellow oil.

HPLC (254 nm, System A): t_{R}=15.38 min, HPLC (254 nm, System B): t_{R}=14.58 min; ESI-MS: m/z=371.04 [M]⁺

### (1S,3R,4S)-Quinuclidin-3-yl(2-(thiophen-2-yl)phenyl)carbamate (REF 2)

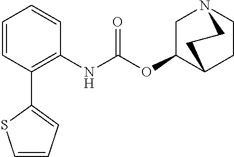 [SMILES: O=C(Nc1ccccc1-c1cccs1)O[C@H]1C[N@]2CC[C@H]1CC2]

Compound REF 2 was prepared according to step 2 of general procedure 4 using a solution of intermediate 38 and R-(−)-3-quinuclidinol, stirring the reaction mixture at 60° C. for 16 h. Purification by preparative HPLC (column 1, eluent: CH₃CN/H₂O +0.1% HCO₂H) afforded the formate salt of REF 2 (40.9 mg, 49%) as yellow oil.

¹H NMR (400 MHz, Methanol-d₄): δ (ppm)=8.53 (s, 1H), 7.66-7.39 (m, 3H), 7.38-7.21 (m, 3H), 7.12 (dd, J=5.2, 3.6 Hz, 1H), 5.00-4.93 (m, 1H), 3.66-3.44 (m, 1H), 3.27-2.69 (m, 5H), 2.40-2.02 (m, 2H), 2.02-1.89 (m, 1H), 1.89-1.47 (m, 2H).

¹³C NMR (151 MHz, Methanol-d₄): δ (ppm)=170.34, 155.90, 141.31, 135.26, 131.16, 129.41, 128.46, 127.52, 127.30, 69.67, 54.76, 47.58, 46.68, 25.61, 21.83, 18.26.

HPLC (254 nm, System A): t_{R}=15.25 min, HPLC (254 nm, System B): t_{R}=14.47 min; ESI-MS: m/z=329.04 [M+H]⁺

### (1S,3R,4S)-1-Methyl-3-(((2-(thiophen-2-yl)phenyl)carbamoyl)oxy)quinuclidin-1-ium iodide (REF 3)

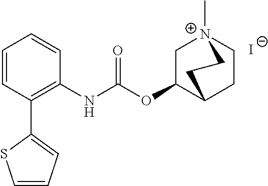 [SMILES: C[N@+]12CC[C@H](CC1)[C@@H](OC(=O)Nc1ccccc1-c1cccs1)C2 ; [I-]]

Compound REF 2 was dissolved in 0.1 mL methanol and the solution was added to a saturated aqueous NaHCO₃ solution. The mixture was extracted three times with chloroform. The combined organic layer was dried with MgSO₄, filtered and the solvent was removed under reduced pressure. The residue was dissolved in dry CH₂Cl₂ (1 mL) and methyl iodide (2.3 eq.) was added under argon atmosphere. The mixture was stirred for eight hours at room temperature. The solvent was removed under reduced pressure to obtain REF 3 (15.2 mg, 71%) as yellow solid.

HPLC (254 nm, System A): t_{R}=15.35 min, HPLC (254 nm, System B): t_{R}=14.98 min; ESI-MS: m/z=343.09 [M]⁺

Receptor Binding Experiments

The affinities of the test compounds towards the human M2 and M3 receptor were determined using homogenates of membranes as described previously (Hübner, H.; Haubmann, C.; Utz, W.; Gmeiner, P., Conjugated enynes as nonaromatic catechol bioisosteres: synthesis, binding experiments, and computational studies of novel dopamine receptor agonists recognizing preferentially the D(3) subtype. J Med Chem 2000, 43, 756-762; Tschammer, N.; Elsner, J.; Goetz, A.; Ehrlich, K.; Schuster, S.; Ruberg, M.; Kühhorn, J.; Thompson, D.; Whistler, J.; Hübner, H.; Gmeiner, P., Highly potent 5-aminotetrahydropyrazolopyridines: enantioselective dopamine D3 receptor binding, functional selectivity, and analysis of receptor-ligand interactions. J Med Chem 2011, 54, 2477-2491). In brief, HEK293T cells were transiently transfected with the cDNA of the appropriate receptor using a solution of linear polyethyleneimine in PBS. Receptor binding experiments were performed in 96-well plates using homogenates of the corresponding receptor at a receptor density (Bₘₐₓ) of 1400±140 fmol/mg and 2200±530 fmol/mg, a protein concentration of 5-10 μg/test tube and 2-10 μg/test tube, and a K_{D} value of 0.20±0.018 nM and 0.086±0.005 nM for M2R and M3R, respectively together with the radioligand [³H]N-methyl-scopolamine bromide (PerkinElmer, Rodgau, Germany). Unspecific binding was determined in the presence of 10 μM atropine. Protein concentration was established by the method of Lowry using bovine serum albumin as standard (Lowry, O. H.; Rosebrough, N. J.; Farr, A. L.; Randall, R. J., Protein measurement with the Folin phenol reagent. J Biol Chem 1951, 193, 265-275). Resulting competition curves were analyzed by nonlinear regression using algorithms for one-site competition.

The kinetic binding assay was performed according to literature (Guo, D.; van Dorp, E. J.; Mulder-Krieger, T.; van Veldhoven, J. P.; Brussee, J.; ljzerman, A. P; Heitman, L. H., Dual-point competition association assay: a fast and high-throughput kinetic screening method for assessing ligand-receptor binding kinetics. J Biomol Screen 2013, 18, 309-320). The analysis of time-dependent binding was done using the algorithms for kinetics of competitive binding in PRISM (San Diego, Calif., US).

1. A compound of formula (I), optionally in the form of a pharmaceutically acceptable salt, solvate, polymorph, tautomer, racemate, enantiomer or diastereomer or mixture thereof, 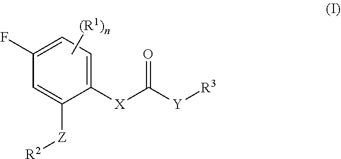
wherein
X is selected from —N(H)— and —C(H)(OH)—;
Y is selected from —O— and —N(H)—;
Z is selected from a bond and -(ethynylene)-;
R¹ is selected from -halogen, —CN, —CF₃, —C₁₋₆-alkyl, —OH and —O—C₁₋₆-alkyl, preferably —F;
n is an integer of 0 to 2;
R² is selected from -(optionally substituted aryl), -(optionally substituted heteroaryl) and -(optionally substituted cycloalkyl), wherein the one or more optional substituent(s) of the aryl, heteroaryl and cycloalkyl are selected from -halogen, —CN, —CF₃, —C₁₋₆-alkyl, —OH, —O—C₁₋₆-alkyl, —NH₂, —N(H)(C₁₋₆-alkyl) and —N(C₁₋₆-alkyl)₂; preferably selected from -halogen, —CN, —OMe and -methyl;
R³ is a -(non-aromatic, optionally bridged, optionally substituted heterocyclic ring having 4 to 7 ring carbon atoms and 1 to 3 heteroatoms selected from N, O and S, including at least one nitrogen, in the ring, and optionally 1 to 3 carbon atoms and 0 to 2 heteroatoms selected from N, O and S in the bridge), wherein the bridge may be saturated or unsaturated and may contain an oxirane moiety, if the heterocyclic ring is a monocyclic ring without any bridge, the heterocyclic ring may be bonded to Y via a methylene group, and
wherein the one or more optional substituent is/are independently selected from —halogen, —(C1-6-alkyl) and —(OH);
wherein the following compounds are disclaimed

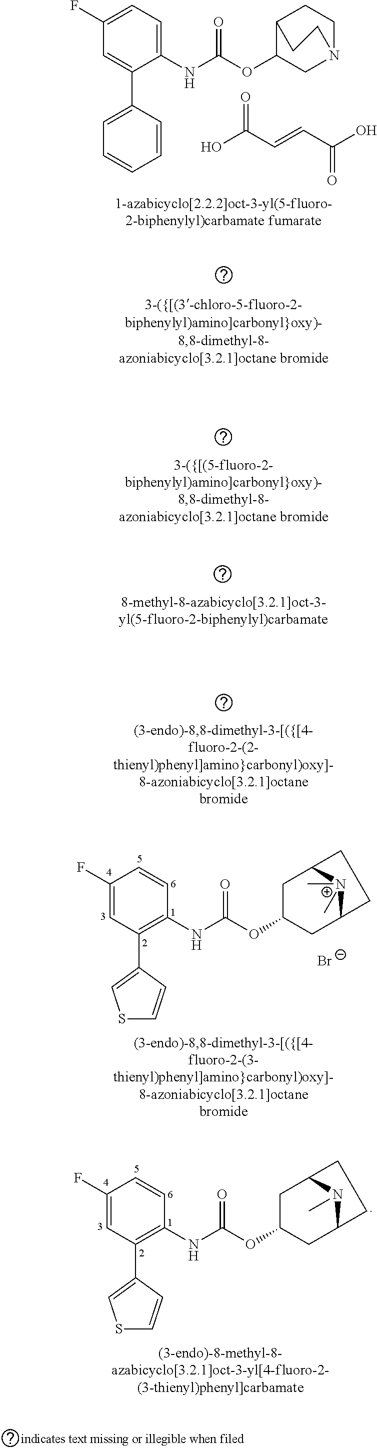

2. The compound according to claim 1, wherein R³ is selected from 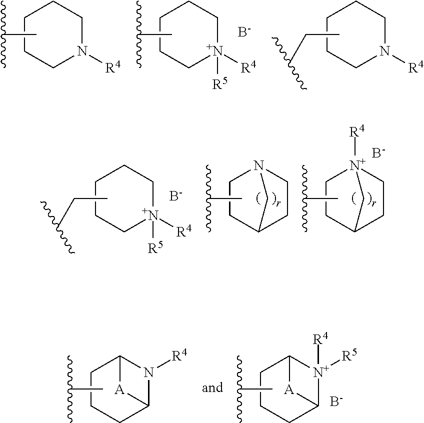
wherein
R⁴ is selected from H and C₁₋₆-alkyl;
R⁵ is selected from H and C₁₋₆-alkyl;
r is an integer from 1 or 2;
A is selected from 1,2-ethylene (—CH₂—CH₂—), 1,2-ethenylene (—CH═CH—), and 2,3-oxiranylene 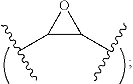
and
B is a physiologically acceptable anion.

3. The compound according to claim 2, wherein R³ is selected from 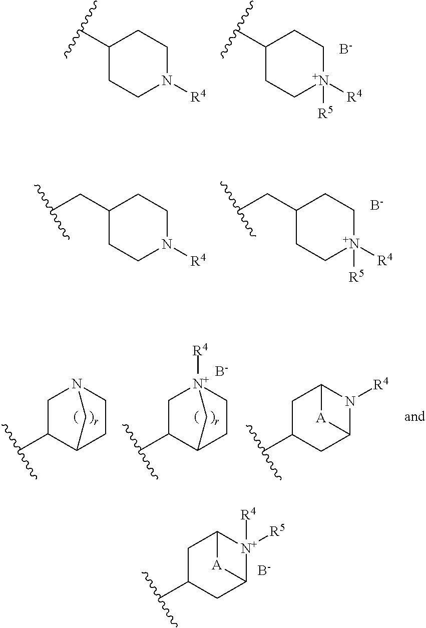
wherein R⁴, R⁵, r, A and B are as defined in claim 2.

4. The compound according to claim 2, wherein R² is not -(optionally substituted phenyl), if Z is a bond, X is —N(H)—, Y is —O— and R³ is 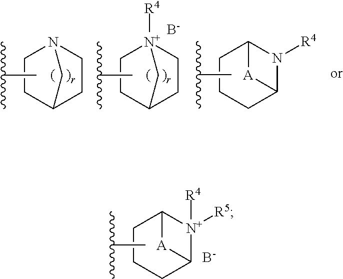
R² is not -(optionally substituted thienyl) if Z is a bond, X is —N(H)—, Y is —O— and R³ is

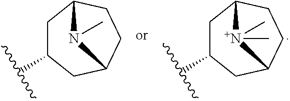

5. The compound according to claim 1, wherein Z is a bond.

6. The compound according to claim 1, wherein X is NH.

7. The compound according to claim 1, wherein Y is O.

8. The compound according to claim 1, wherein n is O.

9. The compound according to claim 1, wherein R² is selected from phenyl, thienyl, furanyl, thiazolyl, pyridyl, pyrimidyl, benzothienyl, benzofuranyl, indenyl and cyclohexyl, wherein phenyl, thienyl, furanyl, thiazolyl, pyridyl, pyrimidyl, benzothienyl, benzofuranyl, indenyl and cyclohexyl are optionally substituted with one or more groups independently selected from -halogen, —CN, —OMe and -methyl.

10. The compound according to claim 1, wherein R³ is 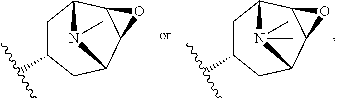 [SMILES: *[C@@H]1C[C@@H]2[C@H]3O[C@H]3[C@H](C1)N2C ; *[C@@H]1C[C@@H]2[C@H]3O[C@H]3[C@H](C1)[N+]2(C)C]
preferably

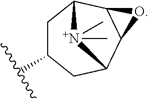 [SMILES: *[C@@H]1C[C@@H]2[C@H]3O[C@H]3[C@H](C1)[N+]2(C)C]

11. The compound according to claim 1, wherein R² is -(optionally substituted heteroaryl), Z is a bond, X is —N(H)—, Y is —O— and R³ is 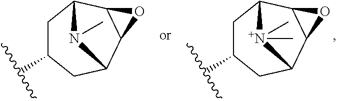 [SMILES: *[C@@H]1C[C@@H]2[C@H]3O[C@H]3[C@H](C1)N2C ; *[C@@H]1C[C@@H]2[C@H]3O[C@H]3[C@H](C1)[N+]2(C)C]
preferably

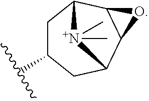 [SMILES: *[C@@H]1C[C@@H]2[C@H]3O[C@H]3[C@H](C1)[N+]2(C)C]

12. The compound according to claim 1, wherein the compound of formula (I) is selected from
piperidin-4-ylmethyl (5-fluoro-[1,1′-biphenyl]-2-yl)carbamate,
piperidin-4-ylmethyl (2′-chloro-5-fluoro-[1,1′-biphenyl]-2-yl)carbamate,
piperidin-4-ylmethyl (4′,5-difluoro-[1,1′-biphenyl]-2-yl)carbamate,
piperidin-4-ylmethyl (4′-chloro-5-fluoro-[1,1′-biphenyl]-2-yl)carbamate,
piperidin-4-ylmethyl (4′-bromo-5-fluoro-[1,1′-biphenyl]-2-yl)carbamate,
piperidin-4-ylmethyl (4′-cyano-5-fluoro-[1,1′-biphenyl]-2-yl)carbamate,
piperidin-4-ylmethyl (5-fluoro-4′-methoxy-[1,1′-biphenyl]-2-yl)carbamate,
piperidin-4-ylmethyl (3′,4′-dichloro-5-fluoro-[1,1′-biphenyl]-2-yl)carbamate,
piperidin-4-ylmethyl (3′,4′,5,5′-tetrafluoro-[1,1′-biphenyl]-2-yl)carbamate,
(1S,3R,4S)-quinuclidin-3-yl (S)-2-(5-fluoro-[1,1′-biphenyl]-2-yl)-2-hydroxyacetate,
(1S,3R,4S)-quinuclidin-3-yl (R)-2-(5-fluoro-[1,1′-biphenyl]-2-yl)-2-hydroxyacetate,
(1S,3R,4S)-3-(((5-fluoro-[1,1′-biphenyl]-2-yl)carbamoyl)oxy)-1-methylquinuclidin-1-ium trifluoroacetate,
1-(5-fluoro-[1,1′-biphenyl]-2-yl)-3-((1S,3R,4S)-quinuclidin-3-yl)urea,
(1S,3R,4S)-3-(3-(5-fluoro-[1,1′-biphenyl]-2-yl)ureido)-1-methylquinuclidin-1-ium trifluoroacetate,
(1S,3R,4S)-quinuclidin-3-yl (4,5-difluoro[2-phenyl]-phen-1-yl)carbamate,
(1S,3R,4S)-quinuclidin-3-yl (3,4,5-trifluoro[2-phenyl]-phen-1-yl)carbamate,
(1S,3R,4S)-quinuclidin-3-yl (3′,4′-dichloro-5-fluoro-[1,1′-biphenyl]-2-yl)carbamate,
(1S,3R,4S)-quinuclidin-3-yl (4-fluoro-2-(thiophen-2-yl)phenyl)carbamate,
(1S,3R,4S)-3-(((4-fluoro-2-(thiophen-2-yl)phenyl)carbamoyl)oxy)-1-methylquinuclidin-1-ium formate,
(1S,3R,4S)-quinuclidin-3-yl (2-(3-bromothiophen-2-yl)-4-fluorophenyl)carbamate,
(1S,3R,4S)-quinuclidin-3-yl (4-fluoro-2-(3-methylthiophen-2-yl)phenyl)carbamate,
(1S,3R,4S)-quinuclidin-3-yl (4-fluoro-2-(4-methylthiophen-2-yl)phenyl)carbamate,
(1S,3R,4S)-quinuclidin-3-yl (4-fluoro-2-(5-methylthiophen-2-yl)phenyl)carbamate,
(1S,3R,4S)-quinuclidin-3-yl (4-fluoro-2-(thiophen-3-yl)phenyl)carbamate,
(1S,3R,4S)-3-(((4-fluoro-2-(thiophen-3-yl)phenyl)carbamoyl)oxy)-1-methylquinuclidin-1-ium formate,
(1S,3R,4S)-quinuclidin-3-yl (4-fluoro-2-(5-methylthiophen-3-yl)phenyl)carbamate,
(1S,3R,4S)-quinuclidin-3-yl (2-(3-bromofuran-2-yl)-4-fluorophenyl)carbamate,
(1S,3R,4S)-quinuclidin-3-yl (4-fluoro-2-(5-methylfuran-2-yl)phenyl)carbamate,
(1S,3R,4S)-quinuclidin-3-yl (4-fluoro-2-(furan-3-yl)phenyl)carbamate,
(1S,3R,4S)-quinuclidin-3-yl (4-fluoro-2-(thiazol-5-yl)phenyl)carbamate,
(1S,3R,4S)-quinuclidin-3-yl (4-fluoro-2-(pyridin-4-yl)phenyl)carbamate,
(1S,3R,4S)-quinuclidin-3-yl (4-fluoro-2-(pyrimidin-5-yl)phenyl)carbamate,
(1R,3R,5S)-8-methyl-8-azabicyclo[3.2.1]octan-3-yl (4-fluoro-2-(thiophen-2-yl)phenyl)carbamate,
(1R,3S,5S)-8-methyl-8-azabicyclo[3.2.1]oct-6-en-3-yl (4-fluoro-2-(thiophen-2-yl)phenyl)carbamate,
(1R,2R,4S,5S,7S)-9-methyl-3-oxa-9-azatricyclo[3.3.1.0^{2,4}]nonan-7-yl (4-fluoro-2-(thiophen-2-yl)phenyl)carbamate,
(1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate,
(1S,3R,4S)-quinuclidin-3-yl (2-(benzo[b]thiophen-2-yl)-4-fluorophenyl)carbamate,
(1S,3R,4S)-quinuclidin-3-yl (2-(benzo[b]thiophen-3-yl)-4-fluorophenyl)carbamate,
(1S,3R,4S)-quinuclidin-3-yl (2-(benzofuran-2-yl)-4-fluorophenyl)carbamate,
(1S,3R,4S)-quinuclidin-3-yl (4-fluoro-2-(1H-inden-3-yl)phenyl)carbamate,
(1S,3R,4S)-quinuclidin-3-yl (4-fluoro-2-(phenylethynyl)phenyl)carbamate,
(1S,3R,4S)-quinuclidin-3-yl (2-(cyclohexylethynyl)-4-fluorophenyl)carbamate,
(1R,2R,4S,5S,7S)-7-(((4-fluoro-2-(5-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(4-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(3-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(thiophen-3-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate,
(1R,2R,4S,5S,7S)-7-(((2-(benzo[b]thiophen-3-yl)-4-fluorophenyl)carbamoyl)oxy)-9,9-dimethyl-3-oxa-9-azatricyclo[3.3.1.0^{2,4}]nonan-9-ium formate, and
(1R,2R,4S,5S,7S)-9-methyl-3-oxa-9-azatricyclo[3.3.1.0^{2,4}]nonan-7-yl (5-fluoro-[1,1′-biphenyl]-2-yl)carbamate;
wherein the compound of formula (I) is more preferably selected from
(1R,2R,4S,5S,7S)-9-methyl-3-oxa-9-azatricyclo[3.3.1.0^{2,4}]nonan-7-yl (4-fluoro-2-(thiophen-2-yl)phenyl)carbamate,
(1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(5-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(4-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(3-methylthiophen-2-yl)phenyl)carbamoyl)oxy)-9,9-dimethyl-3-oxa-9-azatricyclo[3.3.1.0^{2,4}]nonan-9-ium formate,
(1R,2R,4S,5S,7S)-7-(((4-fluoro-2-(thiophen-3-yl)phenyl)carbamoyl)oxy)-9,9-dimethyl-3-oxa-9-azatricyclo[3.3.1.0^{2,4}]nonan-9-ium formate, and
(1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate,
and
wherein the compound of formula (I) is most preferably selected from
(1R,2R,4S,5S,7S)-7-(((4-fluoro-2-(thiophen-2-yl)phenyl)carbamoyl)oxy)-9,9-dimethyl-3-oxa-9-azatricyclo[3.3.1.0^{2,4}]nonan-9-ium formate and
(1R,2R,4S,5S,7S)-7-(((2-(benzo[b]thiophen-2-yl)-4-fluorophenyl)carbamoyl)oxy)-9,9-dimethyl-3-oxa-9-azatricyclo[3.3.1.0^{2,4}]nonan-9-ium formate.

13. A pharmaceutical composition comprising: a compound according to claim 1, optionally in the form of a pharmaceutically acceptable salt, solvate, polymorph, tautomer, racemate, enantiomer or diastereomer or mixture thereof,
and optionally one or more pharmaceutically acceptable excipient(s) and/or carrier(s).

14. (canceled)

15. A method of treating, ameliorating or preventing asthma, chronic obstructive pulmonary disease (COPD), chronic obstructive lung disease, chronic bronchial asthma, chronic bronchitis, bronchopulmonary dysplasia (BPD), chronic airway obstruction, fibroid lung, diffuse panbronchiolitis, bronchiectasis, chronic respiratory obstruction, pulmonary fibrosis, pulmonary emphysema and allergic rhinitis, idiopathic interstitial pneumonia, urinary incontinence and/or cognitive disorders, the method comprising administering to a patient in need thereof an effective amount of a compound according to claim 1.
